(19) United States
(12) Patent Application Publication
(10) Pub. No.: US 2022/0154222 A1
(43) Pub. Date: May 19, 2022

(54) NOVEL NUCLEIC ACID MODIFIERS

(71) Applicants: THE BROAD INSTITUTE, INC., Cambridge, MA (US); THE BRIGHAM AND WOMEN'S HOSPITAL, INC., Boston, MA (US)

(72) Inventors: Amit CHOUDHARY, Boston, MA (US); Veronika SHOBA, Cambridge, MA (US); Sreekanth VEDAGOPURAM, Boston, MA (US)

(21) Appl. No.: 17/439,159

(22) PCT Filed: Mar. 13, 2020
(86) PCT No.: PCT/US2020/022756

§ 371 Date: Sep. 14, 2021

Related U.S. Application Data
(60) Provisional application No. 62/818,631, filed on Mar. 14, 2019.

(51) Int. Cl. C12N 15/90; C12N 9/22; C12N 15/10; C12N 15/11

(52) U.S. Cl. CPC C12N 15/902; C12N 9/22; C12N 2310/3181; C12N 15/111; C12N 2310/20; C12N 15/102

(57) ABSTRACT

The present inventions generally relate to site-specific delivery of nucleic acid modifiers and includes novel DNA-binding proteins and effectors that can be rapidly programmed to make site-specific DNA modifications. The present inventions also provide a synthetic all-in-one genome editor (SAGE) systems comprising designer DNA sequence readers and a set of small molecules that induce double-strand breaks, enhance cellular permeability, inhibit NHEJ and activate HDR, as well as methods of using and delivering such systems.

### CROSS-REFERENCE TO RELATED APPLICATIONS

This application claims the benefit of U.S. Provisional Application No. 62/818,631 filed Mar. 14, 2019. The entire contents of the above-identified application is hereby fully incorporated herein by reference.

### REFERENCE TO AN ELECTRONIC SEQUENCE LISTING

The contents of the electronic sequence listing (BROD-2580WP_ST25.txt”; Size is 4 Kilobytes and it was created on Mar. 4, 2020) is herein incorporated by reference in its entirety.

### TECHNICAL FIELD

The subject matter disclosed herein is generally directed to nucleic acid modifiers with novel DNA readers and effectors that can be rapidly programmed to make site-specific DNA modifications.

### BACKGROUND

Precise genome targeting technologies that can increase efficiency of repair is highly desirable in disease models and therapies. Moreover, precise editing technologies could enable systematic reverse engineering of causal genetic variations by allowing selective perturbation of individual genetic elements, as well as to advance synthetic biology, biotechnological, and medical applications.

CRISPR-Cas9 from S. pyogenes was evolved for rapid and efficient destruction of phage DNA but lacks the functionalities needed for precision genome edits. Following a double-strand break in a knock-in experiment, an exogenously supplied single-stranded oligo donor (ssODN) is integrated at the break site. This integration can be facilitated if the ssODN is readily available at the break site. Further, local inhibition of the NHEJ pathway and/or local activation of HDR at the strand-break site can also tip the balance in favor of DNA recombination. Several small-molecule inhibitors of NHEJ pathway and HDR activators have been reported. However, the mutagenicity and toxicity of genome-wide NHEJ inhibition or HDR activation severely limit the utility of such molecules.

There is also a need to improve homology-directed repair (HDR) efficiency, and a need for new genome engineering technologies. Approaches that provide advantages in vivo over CRISPR nucleases would be preferred. Accordingly, systems that provide precise, controlled genetic alterations, HDR activated pathways, efficient cellular delivery and addressing genomic remediation on a scale to match genomic diversity, less sensitivity to proteases/nucleases, ease and cost of production, low toxicity, and/or improved kinetics relative to CRISPR nuclease systems would provide a nucleic acid modifier technology with distinct advantages.

### SUMMARY

In certain example embodiments, an engineered, non-naturally occurring nucleic acid modifying system is provided, comprising a) one or more DNA readers, wherein a first engineered, non-naturally occurring DNA reader, binds a target nucleic acid; and b) one or more effector components, wherein a first effector component is a small molecule and modifies the target nucleic acid. In embodiments, the first DNA reader is a peptide nucleic acid (PNA) polymer.

The first effector component is in particular embodiments a small molecule synthetic nuclease. In embodiments, the small molecule nuclease facilitates deamination. In certain embodiments, the first effector component is a nitric oxide donor and optionally comprises a second effector component that facilitates diazotization, certain of these systems, the first effector component comprises thioguanosine. In particular embodiments, the system can further comprise a nucleophile, in particular embodiments, the system comprises saccharin, sulfonic acids and/or other nucleophiles, comprising sulfites, bisulfites, thiols, selenides, phosphates, phosphites, phosphides, chloride, bromide, iodide, thiocyanate and their analogs.

In embodiments, the first effector component is a diazonium ion donor. In certain embodiments, the first effector component comprises a triazabutadiene. In certain systems the first effector component is a ruthenium catalyst, optionally, Ruthenium (II) hydride. In particular embodiments, the ruthenium (II) hydride is conjugated to the DNA reader and further comprises an amine donor.

The first effector can comprise a 1,2-cyclodienone, 9,10-phenanthrenedione, 1,2-anthracenedione, 2,3-benzofurandione, indole-2,3-dione, 1,2-acenaphthylenedione or any of their derivatives. In particular embodiments, the first effector component can comprise a catalyst, optionally an oxidation catalyst. In certain instances, the catalyst is attached in close proximity on the first DNA reader, a second DNA reader, or on an optionally provided guide RNA.

The effector component can comprise an epoxide, or a bisulfate donor, optionally comprising a second effector component comprising a quarternary amine.

In some embodiments, the first effector component is a deaminator and further comprises UV light.

Optionally, the DNA reader or effector component comprises a PEG linker comprising one or more functional groups. In embodiments, the DNA reader comprises a linker comprising disulfide, products of azide/alkyne [3+2] cycloaddition, amide, carbamate, ester, urea, thiourea, for the attachment of the one or more effector components.

In embodiments, the first effector component is linked to the first DNA reader. In embodiments, the first effector component is covalently linked to the first DNA reader.

In certain instances, the system can further comprise a second DNA reader and a second effector component. In some embodiments, the first effector component is covalently linked to the first DNA reader and the second effector component is covalently linked to the second DNA reader. The first and second DNA readers can comprise PNA polymers. In embodiments, the first effector component is an inactive small molecule synthetic nuclease and the second effector component is a trigger reagent, wherein the trigger reagent activates the small molecule synthetic nuclease.

In some instances, the synthetic nuclease is a single strand breaking small molecule. In certain instances, the one or more effector components comprises the first effector component, a second effector component, a third effector component, and a fourth effector component, which in embodiments the one or more DNA readers comprises the first DNA reader and a second DNA reader that are PNA polymers, and the first, second, third, and fourth effector component are small molecule single strand breaking synthetic nucleases. The first and second synthetic nucleases can be linked to the first PNA polymer, and the third and fourth synthetic nucleases are linked to the second PNA polymer. Any of the systems disclosed can further comprise one or more single-stranded oligo donors (ssODNs).

In embodiments, the systems can further comprise one or more NHEJ inhibitors and/or one or more HDR activators. In embodiments, the NHEJ inhibitor is an inhibitor of DNA ligase IV, KU70, or KU80. The NHEJ inhibitor in some preferred embodiments is a small molecule. The NHEJ inhibitor in some instances is selected from the group consisting of SCR7-G, KU inhibitor, and analogs thereof.

In embodiments, the systems can further comprise one or more HDR activators, which in certain embodiments is a small molecule.

In embodiments, the target nucleic acid comprises chromosomal DNA, mitochondrial DNA, viral, bacterial, or fungal DNA. In other embodiments, the target nucleic acid comprises viral, bacterial, or fungal RNA.

The systems disclosed herein can further comprise a deamination enhancer, optionally wherein the enhancer is UV-light. Delivery enhancers, including cellular permeability enhancers, can be included in the systems disclosed.

Methods of utilizing the systems and compositions as disclosed herein are provided. Methods of precise genome editing in a cell or tissue, are disclosed, comprising delivering a systems as disclosed to the cell or tissue. In embodiments, the system is delivered using nanoparticles. In particular embodiments, the nanoparticles are selected from poly(lactic co-glycolic acids) (PLGA) nanoparticles, lipid based nanoparticles, PLGA/PLA nanoparticles, mixed poly amine-PLA conjugate nanoparticles, cationic peptide nanoparticles, anionic peptide nanoparticles, or dendrimer based nanoparticles.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-1C includes schematics for base-pairing facilitated: FIG. 1A transfer of nitro group; FIG. 1B Ru-catalyzed coupling of amines; and FIG. 1C imine formation with 1,2 diketone and following deamination to uracil.

FIG. 2 shows triazabutadienes as efficient donors of diazonium ion.

FIG. 3A-3C shows a schematic for base-pairing facilitated: FIG. 3A transfer of nitro group; FIG. 3B utilization of both amine donor and Ruthenium catalyst attached to the same donor; and FIG. 3C imine formation with 1,2 diketone and following deamination to uracil. The amine donor on the Ruthenium catalyst can be removed and located on the nearby structural unit of PNA.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2_{nd} edition (1989) (Sambrook, Fritsch, and Maniatis); Molecular Cloning: A Laboratory Manual, 4ₜₕ edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboraotry Manual, 2_{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2_{nd} edition (2011)

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### OVERVIEW

Embodiments disclosed herein provide an engineered, non-naturally occurring nucleic acid modifying system comprising one or more DNA readers and one or more effector components. In some embodiments, the activity of synthetic nucleases can be masked using pro-drug strategies enabling tissue-specific activation of the system. Some synthetic nucleases require specific triggers and others can be split into two components, affording additional control of specificity and activity of the gene editing systems. Display of additional functionalities can also be achieved, for example, effector components comprising ssODNs, NHEJ inhibitors or HDR activators for precise genome edits.

In some embodiments, the engineered nucleic acid modifying systems can be tuned for varying potencies, including low (>10 μM), medium (0.5-10 μM), and high (<1 nM) with single or double-strand cleavage activity.

In an aspect, the engineered nucleic acid modifying systems provide a molecule or molecules that bind target nucleic acid; and an effector component that modifies, directs breaks, or induces breaks in target nucleic acid. Advantageously the target nucleic acids can include DNA or RNA, for example chromosomal or mitochondrial DNA, viral, bacterial or fungal DNA or viral bacterial, or fungal RNA.

A target sequence may comprise any polynucleotide, such as DNA or RNA polynucleotides. In some embodiments, a target sequence is located in the nucleus or cytoplasm of a cell, and may include nucleic acids in or from mitochondrial, organelles, vesicles, liposomes or particles present within the cell.

In certain embodiments, the effector components can be linked, e.g., covalently conjugated, to the one or more DNA readers. In an aspect, the first effector component can be linked, for example using a PEG linker or other suitable linker, to the first DNA reader. For example, the first effector component can be covalently linked to the first DNA reader. The first effector component can comprise one or more of maleimide, azide, or alkyne functional groups and the first DNA reader, effector molecule or guide RNA can optionally comprise a PEG linker comprising one or more disulfides, products of azide/alkyne [3+2] cycloaddition, amides, carbamates, esters, ureas, thioureas and PEG. The PEG linker can be tuned so that the molecule of the systems provided can be located a particular distance from one another, a particular location on the target molecule, and/or from one another. By varying the length of the PEG linker, it is possible to effect the DNA modification close to or away from the PNA binding site, which provides additional flexibility in designing the DNA modification sites.

In further embodiments, the system can comprise a second DNA reader and a second effector component. The first effector component can be covalently linked to the first DNA reader and the second effector component can also be covalently linked to the second DNA reader.

In some embodiments, both the first and second DNA readers are PNA polymers.

In some embodiments, the first effector component can be an inactive small molecule synthetic nuclease and the second effector component can a trigger reagent, wherein the trigger reagent activates the small molecule synthetic nuclease.

In some embodiments, the first effector component comprises a first fragment of a reactive group of a small molecule synthetic nuclease and the second effector component comprises a second fragment of the reactive group of the small molecule synthetic nuclease, wherein the small molecule synthetic nuclease is only active when the first fragment and the second fragment are together.

In further embodiments, the system can comprise a third and a fourth effector component. In some embodiments, both the first and second DNA readers are PNA polymers, and the first, second, third, and fourth effector component are small molecule single strand breaking synthetic nucleases. In other embodiments, the first and second synthetic nucleases are linked to the first PNA polymer, and the third and fourth synthetic nucleases are linked to the second PNA polymer.

In some embodiments, they system can further comprise one or more single-stranded oligo donors (ssODNs). In other embodiments, the system can further comprise one or more NHEJ inhibitors and/or one or more HDR activators.

### DNA Reader

The systems and methods disclosed herein comprise one or more DNA readers. In preferred embodiments at least one DNA reader is an engineered, non-naturally occurring DNA reader that binds a target nucleic acid, for example a DNA or RNA molecule. The designer nucleic acid sequence readers include target nucleic acid binding molecules designed like CRISPR systems to recognize nucleic acid sequences using a programmable guide.

In certain embodiments, the designer nucleic acid sequence readers comprise one or more peptide nucleic acids (PNAs) polymers. The nucleic acid sequence readers further include readers designed like Transcription Activator-Like Effectors (TALEs) to recognize DNA using two variable amino acid residues for each base being recognized. The invention employs peptidomimetics (e.g., unnatural amino acids, peptoids) and commonly employed chemistries for secondary structure pre-organization (e.g., “stapling,” side-chain crosslinking, hydrogen-bond surrogating) to miniaturize a TALE-like system providing nucleotide sequence readers that are proteolytically and chemically stable.

In certain embodiments, the first DNA reader is a peptide nucleic acid (PNA) polymer, or transcript activator-like effector (TALE). In certain embodiments, the first DNA reader is a PNA polymer.

In some embodiments, the nucleic acid binding domain may comprise at least five or more Transcription activator-like effector (TALE) monomers and at least one or more half-monomers specifically ordered to target the genomic locus of interest linked to at least one or more effector domains are further linked to a chemical or energy sensitive protein. This leads to a change in the sub-cellular localization of the entire polypeptide (i.e. transportation of the entire polypeptide from cytoplasm into the nucleus of the cells) upon the binding of a chemical or energy transfer to the chemical or energy sensitive protein. This transportation of the entire polypeptide from one sub-cellular compartments or organelles, in which its activity is sequestered due to lack of substrate for the effector domain, into another one in which the substrate is present would allow the entire polypeptide to come in contact with its desired substrate (i.e. genomic DNA in the mammalian nucleus) and result in activation or repression of target gene expression.

In an embodiment the nucleic acid modifier comprises Repeat Variable Diresidues (RVDs) of a TALE protein or a portion thereof linked to one or more effector domains.

In an embodiment of a nucleic acid modifier, the nucleic acid binding domain and the effector domain are linked by a linker comprising an inducible linker, a switchable linker, a chemical linker, PEG or (GGGGS)(SEQ ID NO: 10) repeated 1-3 times.

In some general embodiments, the nucleic acid modifying protein is used for multiplex targeting comprises and/or is associated with one or more effector domains. In some more specific embodiments, the nucleic acid modifying protein used for multiplex targeting comprises one or more domains of a catalytically inactive Cas protein, i.e. a dead Cas (“dCas”) protein.

In certain embodiments, the sequence readers comprise or are engineered from zinc finger proteins, meganucleases, argonaute, or other nucleic acid binding domains.

DNA readers in some embodiments can comprise portions of CRISPR-Cas proteins, including one or more functional domains. In each instance, each reader and its effector molecule is smaller than a Cas9 protein, while still allowing for binding of a target nucleic acid and cleavage of the target. In an aspect, the reader is smaller than an SpCas9, or about 1368 amino acids, smaller than an SaCas9, or about 1053 amino acid residues, smaller than a CjCas9, or about 984 amino acid residues.

In an embodiment the nucleic acid binding domain comprises the recognition (REC) lobe of a CRISPR protein linked to one or more effector domains. In an embodiment the nucleic acid modifier comprises domains/subdomains of Class 1, Type II Cas domains, Type V Cas domains, or Type VI domains. In certain example embodiments, the systems comprise a Cas9 linked to one or more effector domains. In an embodiment the nucleic acid modifier comprises domains/subdomains of Cpf1 linked to one or more effector domains. In an embodiment the nucleic acid modifier comprises domains of a Cas13 protein linked to one or more effector domains, and can include, a system as disclosed in PCT/US18/57182 at [0093]-[0187], incorporated herein in its entirety.

In an embodiment of the invention, the nucleic acid binding domain comprises amino binding residues which correspond to amino acids of SpCas9. In an embodiment of the invention, the nucleic acid binding domain comprises one or more of the following domains, whole or in part: RuvC, bridge helix, REC1, and PI. In an embodiment of the invention, the nucleic acid binding domain comprises binding residues which correspond to all or a subset of the following amino acids of SpCas9: Lys30, Lys33, Arg40, Lys44, Asn46, Glu57, Thr62, Arg69, Asn77, Leu101, Ser104, Phe105, Arg115, His116, Ile135, His160, Lys163, Arg165, Gly166, Tyr325, His328, Arg340, Phe351, Asp364, Gln402, Arg403, Thr404, Asn407, Arg447, Ile448, Leu455, Ser460, Arg467, Thr472, Ile473, Lys510, Tyr515, Trp659, Arg661, Met694, Gln695, His698, His721, Ala728, Lys742, Gln926, Val1009, Lys1097, Val1100, Gly1103, Thr1102, Phe1105, Ile1110, Tyr1113, Arg1122, Lys1123, Lys1124, Tyr1131, Glu1225, Ala1227, Gln1272, His1349, Ser1351, and Tyr1356.

Of the amino acids above, SpCas9 amino acids that interact with the guide primarily through the SpCas9 amino acid backbone are Lys33, Lys44, Glu57, Ala59, Leu101, Phe105, Ile135, Gly166, Phe351, Thr404, Ile448, Leu455, Ile473, Trp659, Val1009, Val1100, Gly1103, Phe1105, Ile1110, Tyr1113, Lys1124, Tyr1131, Glu1225, and Ala1227. Modifications of a Cas9 RuvC I catalytic domain allows binding and nickase activity. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A. As a further example, two or more catalytic domains of Cas9 (RuvC I, RuvC II, and RuvC III or the HNH domain) may be mutated to produce a nucleic acid modifying protein substantially lacking all DNA cleavage activity. In some embodiments, a D10A mutation is combined with one or more of H840A, N854A, or N863A mutations to produce a nucleic acid modifying protein substantially lacking all DNA cleavage activity.

In an embodiment of the invention, the nucleic acid binding domain comprises binding residues which correspond to all or a subset Ala59, Arg63, Arg66, Arg70, Arg74, Arg78, Lys50, Tyr515, Arg661, Gln926, and Val1009 of SpCas9, which interact with the sugar-phosphate backbone of the guide in the guide:target duplex. In an embodiment of the invention, the nucleic acid binding domain comprises binding residues which correspond to all or a subset of Leu169, Tyr450, Met495, Asn497, Trp659, Arg661, Met694, Gln695, His698, Ala728, Gln926, and/or Glu1108 of SpCas9, which interact with the sugar-phosphate backbone of the target in the guide:target duplex.

In an embodiment of the invention, the nucleic acid modifying protein comprises at least one HEPN domain, including but not limited to HEPN domains described herein, HEPN domains known in the art, and domains recognized to be HEPN domains by comparison to consensus sequences and motifs. When more than one HEPN domain is utilized, the HEPN domains are preferably mutated relative to wild-type HEPN domains such that nuclease activity is reduced or abolished, but binding is retained. Consensus sequences can be derived from the sequences of the orthologs disclosed in U.S. Provisional Patent Application 62/432,240 entitled “Novel CRISPR Enzymes and Systems,” U.S. Provisional Patent Application 62/471,710 entitled “Novel Type VI CRISPR Orthologs and Systems” filed on Mar. 15, 2017; Reference is further made to East-Seletsky et al. “Two distinct RNase activities of CRISPR-C2c2 enable guide-RNA processing and RNA detection” Nature doi:10/1038/nature19802 and Abudayyeh et al. “C2c2 is a single-component programmable RNA-guided RNA targeting CRISPR effector” bioRxiv doi:10.1101/054742.

### Guide Molecules

The CRISPR-Cas or Cas-Based system described herein can, in some embodiments, include one or more guide molecules. The terms guide molecule, guide sequence and guide polynucleotide, refer to polynucleotides capable of guiding Cas to a target genomic locus and are used interchangeably as in foregoing cited documents such as WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36(4)702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

In some embodiments, the guide molecule is an RNA. The guide molecule(s) (also referred to interchangeably herein as guide polynucleotide and guide sequence) that are included in the CRISPR-Cas or Cas based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net).

A guide sequence, and hence a nucleic acid-targeting guide may be selected to target any target nucleic acid sequence. The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and P A Carr and G M Church, 2009, Nature Biotechnology 27(12): 1151-62).

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence.

In certain embodiments, the crRNA comprises a stem loop, preferably a single stem loop. In certain embodiments, the direct repeat sequence forms a stem loop, preferably a single stem loop.

In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin.

In general, degree of complementarity is with reference to the optimal alignment of the sca sequence and tracr sequence, along the length of the shorter of the two sequences. Optimal alignment may be determined by any suitable alignment algorithm, and may further account for secondary structures, such as self-complementarity within either the sca sequence or tracr sequence. In some embodiments, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In some embodiments, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In some embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and are further contemplated within the context of this invention. Various modifications may be used to increase the specificity of binding to the target sequence and/or increase the activity of the Cas protein and/or reduce off-target effects. Example guide sequence modifications are described in PCT US2019/045582, specifically paragraphs [0178]-[0333]. which is incorporated herein by reference.

### Target Sequences, PAMs, and PFSs

### Target Sequences

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to an RNA polynucleotide being or comprising the target sequence. In other words, the target polynucleotide can be a polynucleotide or a part of a polynucleotide to which a part of the guide sequence is designed to have complementarity with and to which the effector function mediated by the complex comprising the CRISPR effector protein and a guide molecule is to be directed. In some embodiments, a target sequence is located in the nucleus or cytoplasm of a cell.

The guide sequence can specifically bind a target sequence in a target polynucleotide. The target polynucleotide may be DNA. The target polynucleotide may be RNA. The target polynucleotide can have one or more (e.g., 1, 2, 3, 4, 5, 6, 7, 8, 9, 10, etc. or more) target sequences. The target polynucleotide can be on a vector. The target polynucleotide can be genomic DNA. The target polynucleotide can be episomal. Other forms of the target polynucleotide are described elsewhere herein.

The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence (also referred to herein as a target polynucleotide) may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

### PAM and PFS Elements

PAM elements are sequences that can be recognized and bound by Cas proteins. Cas proteins/effector complexes can then unwind the dsDNA at a position adjacent to the PAM element. It will be appreciated that Cas proteins and systems that include them that target RNA do not require PAM sequences (Marraffini et al. 2010. Nature. 463:568-571). Instead, many rely on PFSs, which are discussed elsewhere herein. In certain embodiments, the target sequence should be associated with a PAM (protospacer adjacent motif) or PFS (protospacer flanking sequence or site), that is, a short sequence recognized by the CRISPR complex. Depending on the nature of the CRISPR-Cas protein, the target sequence should be selected, such that its complementary sequence in the DNA duplex (also referred to herein as the non-target sequence) is upstream or downstream of the PAM. In the embodiments, the complementary sequence of the target sequence is downstream or 3′ of the PAM or upstream or 5′ of the PAM. The precise sequence and length requirements for the PAM differ depending on the Cas protein used, but PAMs are typically 2-5 base pair sequences adjacent the protospacer (that is, the target sequence). Examples of the natural PAM sequences for different Cas proteins are provided herein below and the skilled person will be able to identify further PAM sequences for use with a given Cas protein.

The ability to recognize different PAM sequences depends on the Cas polypeptide(s) included in the system. See e.g., Gleditzsch et al. 2019. RNA Biology. 16(4): 504-517

In a preferred embodiment, the CRISPR effector protein may recognize a 3′ PAM. In certain embodiments, the CRISPR effector protein may recognize a 3′ PAM which is 5′H, wherein H is A, C or U.

Further, engineering of the PAM Interacting (PI) domain on the Cas protein may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously. Gao et al, “Engineered Cpf1 Enzymes with Altered PAM Specificities,” bioRxiv 091611; doi: http://dx.doi.org/10.1101/091611 (Dec. 4, 2016). Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.

PAM sequences can be identified in a polynucleotide using an appropriate design tool, which are commercially available as well as online. Such freely available tools include, but are not limited to, CRISPRFinder and CRISPRTarget. Mojica et al. 2009. Microbiol. 155(Pt. 3):733-740; Atschul et al. 1990. J. Mol. Biol. 215:403-410; Biswass et al. 2013 RNA Biol. 10:817-827; and Grissa et al. 2007. Nucleic Acid Res. 35:W52-57. Experimental approaches to PAM identification can include, but are not limited to, plasmid depletion assays (Jiang et al. 2013. Nat. Biotechnol. 31:233-239; Esvelt et al. 2013. Nat. Methods. 10:1116-1121; Kleinstiver et al. 2015. Nature. 523:481-485), screened by a high-throughput in vivo model called PAM-SCNAR (Pattanayak et al. 2013. Nat. Biotechnol. 31:839-843 and Leenay et al. 2016. Mol. Cell. 16:253), and negative screening (Zetsche et al. 2015. Cell. 163:759-771).

As previously mentioned, CRISPR-Cas systems that target RNA do not typically rely on PAM sequences. Instead such systems typically recognize protospacer flanking sites (PFSs) instead of PAMs Thus, Type VI CRISPR-Cas systems typically recognize protospacer flanking sites (PFSs) instead of PAMs. PFSs represents an analogue to PAMs for RNA targets. Type VI CRISPR-Cas systems employ a Cas13. Some Cas13 proteins analyzed to date, such as Cas13a (C2c2) identified from Leptotrichia shahii (LShCas13a) have a specific discrimination against G at the 3′end of the target RNA. The presence of a C at the corresponding crRNA repeat site can indicate that nucleotide pairing at this position is rejected. However, some Cas13 proteins (e.g., LwaCAs13a and PspCas13b) do not seem to have a PFS preference. See e.g., Gleditzsch et al. 2019. RNA Biology. 16(4):504-517. Some Type VI proteins, such as subtype B, have 5′-recognition of D (G, T, A) and a 3′-motif requirement of NAN or NNA. One example is the Cas13b protein identified in Bergeyella zoohelcum (BzCas13b). See e.g., Gleditzsch et al. 2019. RNA Biology. 16(4):504-517. Overall, Type VI CRISPR-Cas systems appear to have less restrictive rules for substrate (e.g., target sequence) recognition than those that target DNA (e.g., Type V and type II).

### Peptide Nucleic Acids (PNAs)

In some preferred embodiments, the DNA reader is a PNA. PNAs act as high-fidelity DNA readers as well as a scaffold for display of synthetic nucleases, further reducing the size compared to that of a typical CRISPR complex. This size reduction will allow facile delivery of multiple systems into a cell type of interest and may even allow highly multiplexed editing. Advantageously, PNAs are resistant to degradation by proteases/nucleases. Second, the synthetic nuclease can be positioned anywhere along the PNA backbone allowing a way to introduce designer cuts—a feature extremely difficult to achieve with CRISPR-associated nucleases. The PNAs can comprise templates which allow the system to introduce indels, introduce new templates in the first paragraph of this section to make clear system can cut to introduce indels or pair with a template to introduce new sequences and that HDR promoters and NHEJ inhibitors may use to favor a particular native cell repair pathway depending on the outcome desired. In some embodiments, templates for HDR can also be directly conjugated to the PNA backbone, enhancing their local concentration and improving the rate of genome integration at the desired site.

PNAs are DNA analogs with neutral synthetic backbone in place of the negatively charged phosphodiester backbone of DNA. This neutral charge allows high-affinity binding to DNA compared to those attained by DNA/DNA or DNA/RNA hybrids. Next-generation PNAs (e.g., γPNA) are preorganized for binding to B-DNA in a sequence-unrestricted manner via Watson-Crick recognition. Additionally, the synthetic backbone of PNAs makes them resistant to proteases/nucleases. Fourth, a PNA/DNA mismatch is more destabilizing than a DNA/RNA mismatch, which could potentially reduce the off-target effects. Finally, efficient in vivo delivery of PNAs has been demonstrated for several disease systems by many groups, as detailed elsewhere herein.

In some preferred embodiments, the nucleic acid modifying system can include two or more PNA molecules. Two PNA molecules can each bear a fragment of a split effector component. For example, for any complex that has two ligands and one can envision an editor where PNA strand bears one ligand while the other bears the remaining complex. In another embodiment, one of the PNA strands can bear an inactive small-molecule while the other PNA will bear the trigger. The effector components, such as single-strand breaking small-molecules, can be positioned at any site on the PNA will be leveraged, essentially allowing the introduction of any type of DNA break.

### Conjugation of Effector Components to DNA Readers

In exemplary embodiments, the DNA reader is conjugated to one or more effector components or molecules. In preferred embodiments, the effector components are chemically conjugated to the DNA readers. In the PNA based genome editor that is envisioned, the PNA serves as the DNA reader that can be customized to target any desired genomic sequence, thereby directing the one or more effector components to a target sequence, where the effector component may act on the target sequence, e.g., induction of DNA strand breaks by synthetic nucleases. The one or more effector molecules may be conjugated to the PNA via covalent conjugation. Previous work on labeling PNA with small molecules have mainly focused on attachment of fluorophores or psoralen (DNA intercalator), chlorambucil (DNA alkylating agent) and camptothecin (Topoisomerase I inhibitor). Fluorescence labeling of PNAs has been achieved at both the N and C terminus by several groups employing diverse strategies. Mayfield and Corey have described labeling of the PNA N terminus with an activated carboxylic acid derivative of the fluorophore as the last step in solid phase synthesis. An alternative is to use custom monomers, such as lysine conjugated with fluorescein at the [symbol] μ-amino group as demonstrated by Lohse et al. and Muse et al. Custom made monomers can also be used in labeling of the C-terminus. For instance Liu et al. achieved C-terminal labeling of PNA by loading the solid support with S-t-butylmercapto-L-cysteine allowing conjugation of the thiol group with maleimido functionalized rhodamine dye directly on solid support. Alternatively, an [symbol] _{μ}-amino-lysine-dye conjugate can be attached to solid support as the first step of PNA synthesis yielding the C-terminus labeled product as demonstrated by Robertson et al. Seitz et al. and Robertson et al. have also described labeling of PNA after its solid phase synthesis. While this is a viable alternative, it involves changes in the PNA structure and is time consuming. Additionally, Kim et al. and Birkedal et al. have described the conjugation of psoralen, chlorambucil and camptothecin to the N-terminus of PNA linked by an ethylene glycol linker. Inspired by these approaches, we will design our small molecules strand breakers to include maleimide, azide or alkyne functional groups while installing a PEG linker with thiol, alkyne or azide functional handles on the PNA respectively to allow for efficient conjugation. Further, by varying the length of the PEG linker, it is possible to effect the DNA cut close to or away from the PNA binding site, which provides additional flexibility in designing the DNA cut sites. To create staggered double stranded breaks on the DNA, two PNA molecules will be conjugated to single strand breakers at both N and C termini designed to bind the target DNA in a staggered fashion. This will effect four staggered cuts in the DNA such that the donor DNA with complementary staggered ends can anneal to bring about precise genomic modification without involving DNA repair pathway. Linkers as disclosed herein, for example, disulfides, products of azide/alkyne [3+2] cycloaddition, amides, carbamates, esters, ureas, thioureas and PEG, can be used for the attachment of facilitators of deamination reactions, e.g., nucleophiles, catalysts, tertiary amines and other facilitators of deamination reactions.

ssODNs

In some embodiments, the one or more effector components further comprise one or more adaptor oligonucleotides, wherein one adaptor oligonucleotide hybridizes with one single stranded oligodeoxynucleotide (ssODN). In an aspect, an exogenously supplied single-stranded oligo donor (ssODN) is integrated at the break site with integration facilitated if the ssODN is readily available at the break site. Further, local inhibition of the NHEJ pathway and/or local activation of HDR at the strand-break site can also tip the balance in favor of DNA recombination. ssODN are typically >100 nucleotides, up to about 2000 nucleotides, and may have diverse secondary structures, which may make chemical conjugation inefficient. Therefore, short adaptor maleimide-oligonucleotides (˜15 nucleotides) can be conjugated to and hybridized with the long ssODN donor. See, WO 2019/135816 at Examples 8 and 10, specifically incorporated herein by reference for attachment strategies and further discussion of ssODNs, The one or more adaptor oligonucleotides can be at least 10 nucleotides, at least 13 nucleotides, at least 15 nucleotides, or at least 17 nucleotides. In some embodiments, each adaptor oligonucleotide and the hybridizing ssODN have at least 13 overlapping nucleotides. The guide nucleic acid can be a guide RNA molecule. In an aspect, the ssODN can introduce substitutions, deletions, insertions, or a combination thereof, or cause a shift in an open reading frame on the target polynucleotide. The ssODN introduces one or more mutations to the target polynucleotide, introduces or corrects a premature stop codon in the target polynucleotide, disrupts a splicing site, restores or introduces a splicing site, inserts a gene or gene fragment at one or both alleles of a target polynucleotide, or a combination thereof. In certain embodiments, the system can be utilized with a nicking effector component, including a nickase.

The ssODN may be used for editing the target polynucleotide. In an aspect, the ssODN may comprise a first portion that is complementary to the target site, and a second portion that comprises the edit, substitution, deletion, or other mutation desired. In some cases, the ssODN comprises one or more mutations to be introduced into the target polynucleotide. Examples of such mutations include substitutions, deletions, insertions, or a combination thereof. The mutations may cause a shift in an open reading frame on the target polynucleotide. In some cases, the ssODN alters a stop codon in the target polynucleotide. For example, the ssODN polynucleotide may correct a premature stop codon. The correction may be achieved by deleting the stop codon or introduces one or more mutations to the stop codon. In other example embodiments, the ssODN addresses loss of function mutations, deletions, or translocations that may occur, for example, in certain disease contexts by inserting or restoring a functional copy of a gene, or functional fragment thereof, or a functional regulatory sequence or functional fragment of a regulatory sequence. A functional fragment refers to less than the entire copy of a gene by providing sufficient nucleotide sequence to restore the functionality of a wild type gene or non-coding regulatory sequence (e.g. sequences encoding long non-coding RNA). In certain example embodiments, the systems disclosed herein may be used to replace a single allele of a defective gene or defective fragment thereof. In another example embodiment, the systems disclosed herein may be used to replace both alleles of a defective gene or defective gene fragment. A “defective gene” or “defective gene fragment” is a gene or portion of a gene that when expressed fails to generate a functioning protein or non-coding RNA with functionality of a corresponding wild-type gene. In certain example embodiments, these defective genes may be associated with one or more disease phenotypes. In certain example embodiments, the defective gene or gene fragment is not replaced but the systems described herein are used to insert ssODNs that encode gene or gene fragments that compensate for or override defective gene expression such that cell phenotypes associated with defective gene expression are eliminated or changed to a different or desired cellular phenotype. In certain embodiments of the invention, the ssODN may include, but not be limited to, genes or gene fragments, encoding proteins or RNA transcripts to be expressed, regulatory elements, repair templates, and the like. According to the invention, the ssODNs may comprise may comprise left end and right end sequence elements that function with transposition components that mediate insertion.

In certain cases, the ssODN manipulates a splicing site on the target polynucleotide. In some examples, the ssODN disrupts a splicing site. The disruption may be achieved by inserting the polynucleotide to a splicing site and/or introducing one or more mutations to the splicing site. In certain examples, the ssODN may restore a splicing site, and may comprise a splicing site sequence.

### Effector Components

One or more effector components can be included in the systems disclosed herein. In a preferred embodiment a first effector component is a small molecule that modifies a target nucleic acid. In an aspect, the effector component can modify a single base. Utilizing the systems as base editors allows direct conversion of one base or based pair into another, enabling the efficient installation of point mutations. Both cytosine and adenosine comprise exocyclic amines that can be deaminated to alter base pairing, with adenosine deaminated to inosine (which will be read as guanine) and cystosine deamination generating uracil. Accordingly, point mutations can be corrected utilizing the effectors described herein.

In some embodiments, the effector component is a small molecule synthetic nuclease, which, in some embodiments is a single strand breaking molecule, in other embodiments, a double strand breaking small molecule. Effector components can comprise ssODNs, NHEJ inhibitors, or HDR activators.

In some embodiments, the nucleic acid modifying systems will induce four precisely spaced nicks on the genomic DNA, excising ˜20 base pairs fragment and leaving behind high-affinity “sticky ends.” Simultaneously, this system will facilitate delivery of a high-concentration of an exogenous DNA (˜20 base pair) that will hybridize to the sticky ends and be inserted into the genome. Here the fact that the single-strand breaking small-molecules can be positioned at any site on the PNA will be leveraged, essentially allowing the introduction of any type of DNA break. Further small effector components, can be in some embodiments, a small molecule synthetic nuclease, that in some embodiments is selected from the group consisting of diazofluorenes, nitracines, metal complexes, enediyenes, methoxsalen derivatives, daunorubicin derivatives and juglones. Embodiments can include a second, third or fourth effector component, which can be small molecule single strand breaking nucleases, as described in WO 2019/135816 at [0223]-[0229], incorporated herein by reference.

### Classes of Effectors

Nitric oxide Donors—first effector component is a nitric oxide donor. In some embodiments, the nitric oxide donor comprises thioguanosine.

The nucleic acid modifying system can comprise a second effector component when using a nitric oxide donor. In some embodiments, the second effector component facilitates diazotization. In one embodiment, the second effector component can comprise saccharin, sulfonic acids and/or other nucleophiles.

In some embodiments, the first effector component is a diazonium ion donor. In some particular embodiments, the diazonium ion donor comprises a triazabutadiene. In particular embodiments, the triazabutadiene is a ruthenium catalyst. Ruthenium (II) hydride is one preferred ruthenium catalyst. In particular embodiments, ruthenium (II) hydride is conjugated to the DNA reader and further comprises an amine donor.

In one embodiment, the first effector is a 1,2 diketonecyclodiene derivative. In particular embodiments, the system further comprises an oxidation catalyst, which can optionally be attached in close proximity on the first DNA reader. Close proximity on the DNA reader allows for a spatial proximity effective to facilitate reaction, in this case, catalyze the reaction.

In some embodiments, the first effector component is an epoxide.

In some embodiments, the first effector component is a bisulfate donor, optionally comprising a second effector component comprising a quarternary amine.

In some embodiments, the first effector component is a deaminator and further comprises UV light.

### HDR Enhancement Using Systems

NHEJ inhibitors and HDR activators can be displayed on the synthetic nucleic acid modifiers to enhance HDR as discussed. Simultaneous display of NHEJ inhibitors/HDR activators and DNA strand breakers requires multiple attachment sites on the PNA. The peptide backbone of the PNA provides such additional sites of attachment, including using functionalized PEG linkers (alkyne, azide, cyclooctyne etc.) that are commercially available can be employed for conjugation of NHEJ inhibitors at the [symbol] ≥position. Functionalization of PNA at the [symbol] ≥position by attachment of (R)-diethylene glycol miniPEG (MP) transforms a randomly folded PNA into a right-handed helix providing right handed helical, R-MP [symbol] ≥PNA oligomers that hybridize to DNA and RNA with greater affinity and sequence selectivity than the parental PNA oligomers. Further, the miniPEG PNA has also been successfully used in ex vivo and in vivo studies for gene editing applications.

In some embodiments, the NHEJ inhibitor is an inhibitor of DNA ligase IV, KU70, or KU80. The NHEJ inhibitor can be a small molecule. For example, the NHEJ inhibitor can be selected from the group consisting of SCR7-G, KU inhibitor, and analogs thereof. In some embodiments, the NHEJ inhibitor is adenovirus 4 E1B55K or E4orf6. In some embodiments, the HDR activator is a small molecule. For example, the HDR activator is RS1 or analogs thereof. The HDR activator can also stimulate RAD51 activity.

### Guide Molecules

The nucleic acid modifying systems described herein can, in some embodiments, include one or more guide molecules. The terms guide molecule, guide sequence and guide polynucleotide, refer to polynucleotides capable of guiding the nucleic acid modifying systems to a genomic locus and are used interchangeably as in foregoing cited documents such as WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36(4)702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

In some embodiments, the guide molecule is an RNA. The guide molecule(s) (also referred to interchangeably herein as guide polynucleotide and guide sequence) that are included in the CRISPR-Cas or Cas based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net).

A guide sequence, and hence a nucleic acid-targeting guide may be selected to target any target nucleic acid sequence. The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence.

In certain embodiments, the crRNA comprises a stem loop, preferably a single stem loop. In certain embodiments, the direct repeat sequence forms a stem loop, preferably a single stem loop.

In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin.

In general, degree of complementarity is with reference to the optimal alignment of the sca sequence and tracr sequence, along the length of the shorter of the two sequences. Optimal alignment may be determined by any suitable alignment algorithm, and may further account for secondary structures, such as self-complementarity within either the sca sequence or tracr sequence. In some embodiments, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In some embodiments, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In some embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and are further contemplated within the context of this invention. Various modifications may be used to increase the specificity of binding to the target sequence and/or increase the activity of the Cas protein and/or reduce off-target effects. Example guide sequence modifications are described in PCT US2019/045582, specifically paragraphs [0178]-[0333]. which is incorporated herein by reference.

### Delivery

In one aspect, the invention provides a particle delivery system comprising a composite virus particle, wherein the composite virus particle comprises a lipid, a virus capsid protein, and at least a portion of a non-capsid protein or peptide. The non-capsid peptide or protein can have a molecular weight of up to one megadalton.

In one embodiment, the particle delivery system comprises a virus particle adsorbed to a liposome or lipid particle or nanoparticle. In one embodiment, a virus is adsorbed to a liposome or lipid particle or nanoparticle either through electrostatic interactions, or is covalently linked through a linker. The lipid particle or nanoparticles (1 mg/ml) dissolved in either sodium acetate buffer (pH 5.2) or pure H2O (pH 7) are positively charged. The isoelectropoint of most viruses is in the range of 3.5-7. They have a negatively charged surface in either sodium acetate buffer (pH 5.2) or pure H2O. The electrostatic interaction between the virus and the liposome or synthetic lipid nanoparticle is the most significant factor driving adsorption. By modifying the charge density of the lipid nanoparticle, e.g. inclusion of neutral lipids into the lipid nanoparticle, it is possible to modulate the interaction between the lipid nanoparticle and the virus, hence modulating the assembly. In one embodiment, the liposome comprises a cationic lipid.

In one embodiment, the liposome of the particle delivery system comprises a CRISPR system component.

In one aspect, the invention provides a delivery system comprising one or more hybrid virus capsid proteins in combination with a lipid particle, wherein the hybrid virus capsid protein comprises at least a portion of a virus capsid protein attached to at least a portion of a non-capsid protein.

In one embodiment, the virus capsid protein of the delivery system is attached to a surface of the lipid particle. When the lipid particle is a bilayer, e.g., a liposome, the lipid particle comprises an exterior hydrophilic surface and an interior hydrophilic surface. In one embodiment, the virus capsid protein is attached to a surface of the lipid particle by an electrostatic interaction or by hydrophobic interaction.

In one embodiment, the particle delivery system has a diameter of 50-1000 nm, preferably 100-1000 nm.

In one embodiment, the particle delivery system comprises a non-capsid protein or peptide, wherein the non-capsid protein or peptide has a molecular weight of up to a megadalton. In one embodiment, the non-capsid protein or peptide has a molecular weight in the range of 110 to 160 kDa, 160 to 200 kDa, 200 to 250 kDa, 250 to 300 kDa, 300 to 400 kDa, or 400 to 500 kDa.

In one embodiment, a composite virus particle of the delivery system comprises a lipid, wherein the lipid comprises at least one cationic lipid.

In one embodiment, the delivery system comprises a lipid particle, wherein the lipid particle comprises at least one cationic lipid.

In one embodiment, a particle of the delivery system comprises a lipid layer, wherein the lipid layer comprises at least one cationic lipid.

As used herein, a “composite virus particle” means a virus particle that includes, at a minimum, at least a portion of a virus capsid protein, one or more lipids and a non-capsid protein or peptide. The lipid can be part of a liposome and the virus particle can be adsorbed to the liposome. In certain embodiments, the virus particle is attached to the lipid directly. Alternatively, the virus particle is attached to the lipid via a linker moiety. As used herein, “at least a portion of” means at least 50%, for example, 50%, 55%, 60%, 65%, 70%, 75%, 80%, 85%, 90% or 99%. “At least a portion of”, as it refers to a virus capsid protein or a non-capsid protein, means of a length that is sufficient to allow the two proteins to attach, either directly or via a linker. “At least a portion of”, as it refers to an outer protein or a non-capsid protein, means of a length that is sufficient to allow the two proteins to attach, either directly or via a linker. As used herein, a “lipid particle” is a particle comprised of lipid molecules. As used herein, a “lipid layer” means a layer of lipid molecules arranged side-by-side, preferably with charged groups aligned to one surface. For example, a biological membrane typically comprises two lipid layers, with hydrophobic regions arranged tail-to-tail, and charged regions exposed to an aqueous environment. Using a linker to covalently attach the skilled person from knowledge in the art and this disclosure can obtain 5-100% virus or capsid or virus outer protein or envelope attached to non-capsid or non-virus outer protein or non-envelope protein.

The lipid, lipid particle, or lipid bilayer or lipid entity of the invention can be prepared by methods well known in the art. See Wang et al., ACS Synthetic Biology, 1, 403-07 (2012); Wang et al., PNAS, 113(11) 2868-2873 (2016); Manoharan, et al., WO 2008/042973; Zugates et al., U.S. Pat. No. 8,071,082; Xu et al., WO 2014/186366 A1 (US20160082126). Exemplary compounds are as described in International Patent Publication WO 2019/135816 at [0522]-[0537], incorporated herein by reference.

Xu et provides a way to make a nanocomplex for the delivery of saporin wherein the nanocomplex comprising saporin and a lipid-like compound, and wherein the nanocomplex has a particle size of 50 nm to 1000 nm; the saporin binds to the lipid-like compound via noncovalent interaction or covalent bonding; and the lipid-like compound has a hydrophilic moiety, a hydrophobic moiety, and a linker joining the hydrophilic moiety and the hydrophobic moiety, the hydrophilic moiety being optionally charged and the hydrophobic moiety having 8 to 24 carbon atoms. Xu et al., WO 2014/186348 (US20160129120) provides examples of nanocomplexes of modified peptides or proteins comprising a cationic delivery agent and an anionic pharmaceutical agent, wherein the nanocomplex has a particle size of 50 to 1000 nm, the cationic delivery agent binds to the anionic pharmaceutical agent, and the anionic pharmaceutical agent is a modified peptide or protein formed of a peptide and a protein and an added chemical moiety that contains an anionic group. The added chemical moiety is linked to the peptide or protein via an amide group, an ester group, an ether group, a thioether group, a disulfide group, a hydrazone group, a sulfenate ester group, an amidine group, a urea group, a carbamate group, an imidoester group, or a carbonate group.

In one embodiment, the lipid compound is preferably a bio-reducible material, e.g., a bio-reducible polymer and a bio-reducible lipid-like compound.

In embodiment, the lipid compound comprises a hydrophilic head, and a hydrophobic tail, and optionally a linker.

In one embodiment, the hydrophilic head contains one or more hydrophilic functional groups, e.g., hydroxyl, carboxyl, amino, sulfhydryl, phosphate, amide, ester, ether, carbamate, carbonate, carbamide and phosphodiester. These groups can form hydrogen bonds and are optionally positively or negatively charged, in particular at physiological conditions such as physiological pH.

In one embodiment, the hydrophobic tail is a saturated or unsaturated, linear or branched, acyclic or cyclic, aromatic or nonaromatic hydrocarbon moiety, wherein the saturated or unsaturated, linear or branched, acyclic or cyclic, aromatic or nonaromatic hydrocarbon moiety optionally contains a disulfide bond and/or 8-24 carbon atoms. One or more of the carbon atoms can be replaced with a heteroatom, such as N, O, P, B, S, Si, Sb, Al, Sn, As, Se, and Ge. The lipid or lipid-like compounds containing disulfide bond can be bioreducible.

In one embodiment, the linker of the lipid or lipid-like compound links the hydrophilic head and the hydrophobic tail. The linker can be any chemical group that is hydrophilic or hydrophobic, polar or non-polar, e.g., O, S, Si, amino, alkylene, ester, amide, carbamate, carbamide, carbonate phosphate, phosphite, sulfate, sulfite, and thiosulfate.

The lipid or lipid-like compounds described above include the compounds themselves, as well as their salts and solvates, if applicable. A salt, for example, can be formed between an anion and a positively charged group (e.g., amino) on a lipid-like compound. Suitable anions include chloride, bromide, iodide, sulfate, nitrate, phosphate, citrate, methanesulfonate, trifluoroacetate, acetate, malate, tosylate, tartrate, fumurate, glutamate, glucuronate, lactate, glutarate, and maleate. Likewise, a salt can also be formed between a cation and a negatively charged group (e.g., carboxylate) on a lipid-like compound. Suitable cations include sodium ion, potassium ion, magnesium ion, calcium ion, and an ammonium cation such as tetramethylammonium ion. The lipid-like compounds also include those salts containing quaternary nitrogen atoms. A solvate refers to a complex formed between a lipid-like compound and a pharmaceutically acceptable solvent. Examples of pharmaceutically acceptable solvents include water, ethanol, isopropanol, ethyl acetate, acetic acid, and ethanolamine.

In one embodiment, the lipid, lipid particle or lipid layer of the delivery system further comprises a wild-type capsid protein.

In one embodiment, a weight ratio of hybrid capsid protein to wild-type capsid protein is from 1:10 to 1:1, for example, 1:1, 1:2, 1:3, 1:4, 1:5, 1:6, 1:7, 1:8, 1:9 and 1:10. Further delivery approaches can be used, as disclosed, for example, at [0546]-[0601] in PCT/US18/57182, incorporated herein by reference.

In an aspect, the invention provides a pharmaceutical composition comprising the particle delivery system or the delivery system or the virus particle of any one of the above embodiments or the cell of any one of the above embodiment.

Delivery of systems in vivo can be accomplished by delivery of PNAs using Poly(lactic co-glycolic acids) (PLGA) nanoparticles as detailed elsewhere herein. PLGA is a biodegradable polymer commonly used in drug delivery systems and medical devices. PLGA undergoes hydrolytic degradation into endogenous, non-toxic metabolites (lactic acid and glycolic acid) and has been approved by US Food and Drug Administration (USFDA). Given these attractive properties of PLGA, it is unsurprising that PLGA nanoparticles have been used for cellular delivery of PNAs in several studies. McNeer et al. and Scheifman et al. used PLGA nanoparticles to deliver triplex forming PNAs and donor DNAs for site specific genome editing of CD34+ HPSCs. In another study, McNeer et al. demonstrated the generalizability of this approach by introducing a 6 bp mutation into the CCRS gene in human hematopoietic progenitor cells. Further, they have also demonstrated delivery in the human [symbol] ≥-globin gene in mice reconstituted with human hematopoietic cells as well as in an eGFP reporter mouse model providing evidence of direct, in vivo site specific gene editing by PNA-DNA NPs. Although PGLA nanoparticles are widely used in medicine due to its enhanced biocompatibility, it has limited DNA loading capacity. In order to increase, its oligonucleotide loading capacity, cationic polymers such as poly (beta-amino-esters) (PBAE) have been used in combination with PLGA. Bahal et al. have used single-stranded [symbol] ≥PNA along with DNA donor in PBAE-PLGA nanoparticles to correct a disease causing [symbol] ≤-thalassemia mutation both ex vivo and in a [symbol] ≤-globin/eGFP reporter mouse. Fields et al. used an intranasal delivery route to show increased cellular uptake and gene editing in the lungs of [symbol] ≤-globin/eGFP reporter mouse by PNA and donor DNA encapsulated in PBAE-PLGA NPs compared to PLGA NPs. Further, Mc.Neer and Anandalingam et al. demonstrated the correction of the most prevalent cystic fibrosis transmembrane conductance regulator (CFTR) mutation in human CBFE cells as well as in a mouse model. In the light of these numerous reports of delivery in primary cells and mouse models using PNAs encapsulated in nanoparticles, it is envisioned that the nanoparticle based delivery system to be ideal for intracellular delivery of our small-molecule PNA conjugates. To prepare nanoparticle formulations of small molecule-PNA conjugate and donor DNA the approach described by Bahal et al. will be employed. Briefly, small-molecule PNA conjugate and donor DNA will be encapsulated in PGLA nanoparticles using double emulsion solvent evaporation technique. The first emulsion is formed by dropwise addition of aqueous solution of small molecule-PNA conjugate and donor DNA to a solution containing 50:50 ester-terminated PGLA in dichloromethane, followed by ultrasonication. To form the second emulsion, the first emulsion is added slowly, dropwise to 5% aqueous polyvinyl alcohol and then ultrasonicated. This mixture was then poured into 0.3% aqueous polyvinyl alcohol and stirred at room temperature for 3 hrs to obtain nanoparticles. The nanoparticles are then thoroughly washed and collected by centrifugation, resuspended in water, frozen at −80-∞C. and then lyophilized. Nanoparticles will be resuspended in cell culture medium by vigorous vortexing and water sonication and directly added to the cells. In the event that the donor DNA template gets cleaved when co-encapsulated with the small-molecule strand breaker-PNA conjugate, they will be encapsulated in separate nanoparticles as these have also been shown to yield desired genomic modification albeit to a lower extent. Further description of delivery of PNAs as well as discussion of PNAS are as described in the following references in incorporated herein by reference: Sequence-unrestricted, Watson-Crick recognition of double helical B-DNA by (R)-miniPEG-gammaPNAs. Bahal, R.; Sahu, B.; Rapireddy, S.; Lee, C. M.; Ly, D. H. Chembiochem 2012, 13, 56-60; PNA hybridizes to complementary oligonucleotides obeying the Watson-Crick hydrogen-bonding rules. Egholm, M.; Buchardt, O.; Christensen, L.; Behrens, C.; Freier, S. M.; Driver, D. A.; Berg, R. H.; Kim, S. K.; Norden, B.; Nielsen, P. E. Nature 1993, 365, 566-8; Kinetics for hybridization of peptide nucleic acids (PNA) with DNA and RNA studied with the BIAcore technique. Jensen, K. K.; Orum, H.; Nielsen, P. E.; Norden, B. Biochemistry 1997, 36, 5072-7; Variability in the stability of DNA-peptide nucleic acid (PNA) single-base mismatched duplexes: real-time hybridization during affinity electrophoresis in PNA-containing gels. Igloi, G. L. Proc Natl Acad Sci USA 1998, 95, 8562-7.PMC21115; An introduction to peptide nucleic acid. Nielsen, P. E.; Egholm, M. Curr Issues Mol Biol 1999, 1, 89-104; Thermodynamic comparison of PNA/DNA and DNA/DNA hybridization reactions at ambient temperature. Schwarz, F. P.; Robinson, S.; Butler, J. M. Nucleic Acids Res 1999, 27, 4792-800.PMC148780; The first crystal structures of RNA-PNA duplexes and a PNA-PNA duplex containing mismatches—toward anti-sense therapy against TREDs. Kiliszek, A.; Banaszak, K.; Dauter, Z.; Rypniewski, W. Nucleic Acids Res 2016, 44, 1937-43.PMC4770230; Nanoparticles deliver triplex-forming PNAs for site-specific genomic recombination in CD34+ human hematopoietic progenitors. McNeer, N. A.; Chin, J. Y.; Schleifman, E. B.; Fields, R. J.; Glazer, P. M.; Saltzman, W. M. Mol Ther 2011, 19, 172-80.PMC3017438; Systemic delivery of triplex-forming PNA and donor DNA by nanoparticles mediates site-specific genome editing of human hematopoietic cells in vivo. McNeer, N. A.; Schleifman, E. B.; Cuthbert, A.; Brehm, M.; Jackson, A.; Cheng, C.; Anandalingam, K.; Kumar, P.; Shultz, L. D.; Greiner, D. L.; Saltzman, W. M.; Glazer, P. M. Gene Ther 2013, 20, 658-69.3713483; Site-specific Genome Editing in PBMCs With PLGA Nanoparticle-delivered PNAs Confers HIV-1 Resistance in Humanized Mice. Schleifman, E. B.; McNeer, N. A.; Jackson, A.; Yamtich, J.; Brehm, M. A.; Shultz, L. D.; Greiner, D. L.; Kumar, P.; Saltzman, W. M.; Glazer, P. M. Mol Ther Nucl Acids 2013, 2, e135.PMC3889188; Single-stranded gammaPNAs for in vivo site-specific genome editing via Watson-Crick recognition. Bahal, R.; Quijano, E.; McNeer, N. A.; Liu, Y.; Bhunia, D. C.; Lopez-Giraldez, F.; Fields, R. J.; Saltzman, W. M.; Ly, D. H.; Glazer, P. M. Curr Gene Ther 2014, 14, 331-42.PMC4333085; Modified poly(lactic-co-glycolic acid) nanoparticles for enhanced cellular uptake and gene editing in the lung. Fields, R. J.; Quijano, E.; McNeer, N. A.; Caputo, C.; Bahal, R.; Anandalingam, K.; Egan, M. E.; Glazer, P. M.; Saltzman, W. M. Adv Healthc Mater 2015, 4, 361-6.PMC4339402; Nanoparticles that deliver triplex-forming peptide nucleic acid molecules correct F508del CFTR in airway epithelium. McNeer, N. A.; Anandalingam, K.; Fields, R. J.; Caputo, C.; Kopic, S.; Gupta, A.; Quijano, E.; Polikoff, L.; Kong, Y.; Bahal, R.; Geibel, J. P.; Glazer, P. M.; Saltzman, W. M.; Egan, M. E. Nat Commun 2015, 6, 6952.PMC4480796; In vivo correction of anaemia in beta-thalassemic mice by gammaPNA-mediated gene editing with nanoparticle delivery. Bahal, R.; Ali McNeer, N.; Quijano, E.; Liu, Y.; Sulkowski, P.; Turchick, A.; Lu, Y. C.; Bhunia, D. C.; Manna, A.; Greiner, D. L.; Brehm, M. A.; Cheng, C. J.; Lopez-Giraldez, F.; Ricciardi, A.; Beloor, J.; Krause, D. S.; Kumar, P.; Gallagher, P. G.; Braddock, D. T.; Mark Saltzman, W.; Ly, D. H.; Glazer, P. M. Nat Commun 2016, 7, 13304.PMC5095181 application; Site-specific gene modification by PNAs conjugated to psoralen. Kim, K. H.; Nielsen, P. E.; Glazer, P. M. Biochemistry 2006, 45, 314-23; Targeted gene correction using psoralen, chlorambucil and camptothecin conjugates of triplex forming peptide nucleic acid (PNA). Birkedal, H.; Nielsen, P. E. Artif DNA PNA XNA 2011, 2, 23-32.PMC3116579; Enhancing solid phase synthesis by a noncovalent protection strategy-efficient coupling of rhodamine to resin-bound peptide nucleic acids. Mayfield, L. D.; Corey, D. R. Bioorg Med Chem Lett 1999, 9, 1419-22; Fluorescein-conjugated lysine monomers for solid phase synthesis of fluorescent peptides and PNA oligomers. Lohse, J.; Nielsen, P. E.; Harrit, N.; Dahl, O. Bioconjug Chem 1997, 8, 503-9; Sequence selective recognition of double-stranded RNA at physiologically relevant conditions using PNA-peptide conjugates. Muse, O.; Zengeya, T.; Mwaura, J.; Hnedzko, D.; McGee, D. W.; Grewer, C. T.; Rozners, E. ACS Chem Biol 2013, 8, 1683-6.PMC3745792; Strategies for the synthesis of fluorescently labelled PNA. Liu, X.; Balasubramanian, S. Tetrahedron Lett 2000, 41, 6153-6156; Fluorescent PNA probes as hybridization labels for biological RNA. Robertson, K. L.; Yu, L.; Armitage, B. A.; Lopez, A. J.; Peteanu, L. A. Biochemistry 2006, 45, 6066-74; Convergent strategies for the attachment of fluorescing reporter groups to peptide nucleic acids in solution and on solid phase. Seitz, O.; Kohler, O. Chemistry 2001, 7, 3911-25; Site-directed recombination via bifunctional PNA-DNA conjugates. Rogers, F. A.; Vasquez, K. M.; Egholm, M.; Glazer, P. M. Proc Natl Acad Sci USA 2002, 99, 16695-700.PMC139206; Targeted gene modification of hematopoietic progenitor cells in mice following systemic administration of a PNA-peptide conjugate. Rogers, F. A.; Lin, S. S.; Hegan, D. C.; Krause, D. S.; Glazer, P. M. Mol Ther 2012, 20, 109-18.PMC3255600; Peptide nucleic acid-targeted mutagenesis of a chromosomal gene in mouse cells. Faruqi, A. F.; Egholm, M.; Glazer, P. M. In Proc Natl Acad Sci USA 1998; Vol. 95, p 1398-403. PMCID.

In some embodiments, delivery systems may include, for example, Su X, Fricke J, Kavanagh D G, Irvine D J (“In vitro and in vivo mRNA delivery using lipid-enveloped pH-responsive polymer nanoparticles” Mol Pharm. 2011 Jun. 6; 8(3):774-87. doi: 10.1021/mp100390w. Epub 2011 Apr. 1) which describes biodegradable core-shell structured nanoparticles with a poly(β-amino ester) (PBAE) core enveloped by a phospholipid bilayer shell. These were developed for in vivo mRNA delivery. The pH-responsive PBAE component was chosen to promote endosome disruption, while the lipid surface layer was selected to minimize toxicity of the polycation core. Such are, therefore, preferred for delivering RNA of the present invention.

In one embodiment, nanoparticles based on self-assembling bioadhesive polymers are contemplated, which may be applied to oral delivery of peptides, intravenous delivery of peptides and nasal delivery of peptides, all to the brain. Other embodiments, such as oral absorption and ocular delivery of hydrophobic drugs are also contemplated. The molecular envelope technology involves an engineered polymer envelope which is protected and delivered to the site of the disease (see, e.g., Mazza, M. et al. ACSNano, 2013. 7(2): 1016-1026; Siew, A., et al. Mol Pharm, 2012. 9(1):14-28; Lalatsa, A., et al. J Contr Rel, 2012. 161(2):523-36; Lalatsa, A., et al., Mol Pharm, 2012. 9(6):1665-80; Lalatsa, A., et al. Mol Pharm, 2012. 9(6):1764-74; Garrett, N. L., et al. J Biophotonics, 2012. 5(5-6):458-68; Garrett, N. L., et al. J Raman Spect, 2012. 43(5):681-688; Ahmad, S., et al. J Royal Soc Interface 2010. 7:S423-33; Uchegbu, I. F. Expert Opin Drug Deliv, 2006. 3(5):629-40; Qu, X., et al. Biomacromolecules, 2006. 7(12):3452-9 and Uchegbu, I. F., et al. Int J Pharm, 2001. 224:185-199). Doses of about 5 mg/kg are contemplated, with single or multiple doses, depending on the target tissue.

In one embodiment, nanoparticles that can deliver RNA to a cancer cell to stop tumor growth developed by Dan Anderson's lab at MIT may be used/and or adapted to the CRISPR Cas system of the present invention. In particular, the Anderson lab developed fully automated, combinatorial systems for the synthesis, purification, characterization, and formulation of new biomaterials and nanoformulations. See, e.g., Alabi et al., Proc Natl Acad Sci USA. 2013 Aug. 6; 110(32):12881-6; Zhang et al., Adv Mater. 2013 Sep. 6; 25(33):4641-5; Jiang et al., Nano Lett. 2013 Mar. 13; 13(3):1059-64; Karagiannis et al., ACS Nano. 2012 Oct. 23; 6(10):8484-7; Whitehead et al., ACS Nano. 2012 Aug. 28; 6(8):6922-9 and Lee et al., Nat Nanotechnol. 2012 Jun. 3; 7(6):389-93.

The lipid particles developed by the Qiaobing Xu's lab at Tufts University may be used/adapted to the present delivery system for cancer therapy. See Wang et al., J. Control Release, 2017 Jan. 31. pii: 50168-3659(17)30038-X. doi: 10.1016/j.jconre1.2017.01.037. [Epub ahead of print]; Altmoglu et al., Biomater Sci., 4(12):1773-80, Nov. 15, 2016; Wang et al., PNAS, 113(11):2868-73 Mar. 15, 2016; Wang et al., PloS One, 10(11): e0141860. doi: 10.1371/journal.pone.0141860. eCollection 2015, Nov. 3, 2015; Takeda et al., Neural Regen Res. 10(5):689-90, May 2015; Wang et al., Adv. Healthc Mater., 3(9):1398-403, September 2014; and Wang et al., Agnew Chem Int Ed Engl., 53(11):2893-8, Mar. 10, 2014.

US patent application 20110293703 relates to lipidoid compounds are also particularly useful in the administration of polynucleotides, which may be applied to deliver the CRISPR Cas system of the present invention. In one aspect, the aminoalcohol lipidoid compounds are combined with an agent to be delivered to a cell or a subject to form microparticles, nanoparticles, liposomes, or micelles. The agent to be delivered by the particles, liposomes, or micelles may be in the form of a gas, liquid, or solid, and the agent may be a polynucleotide, protein, peptide, or small molecule. The minoalcohol lipidoid compounds may be combined with other aminoalcohol lipidoid compounds, polymers (synthetic or natural), surfactants, cholesterol, carbohydrates, proteins, lipids, etc. to form the particles. These particles may then optionally be combined with a pharmaceutical excipient to form a pharmaceutical composition.

US Patent Publication No. 20110293703 also provides methods of preparing the aminoalcohol lipidoid compounds. One or more equivalents of an amine are allowed to react with one or more equivalents of an epoxide-terminated compound under suitable conditions to form an aminoalcohol lipidoid compound of the present invention. In certain embodiments, all the amino groups of the amine are fully reacted with the epoxide-terminated compound to form tertiary amines. In other embodiments, all the amino groups of the amine are not fully reacted with the epoxide-terminated compound to form tertiary amines thereby resulting in primary or secondary amines in the aminoalcohol lipidoid compound. These primary or secondary amines are left as is or may be reacted with another electrophile such as a different epoxide-terminated compound. As will be appreciated by one skilled in the art, reacting an amine with less than excess of epoxide-terminated compound will result in a plurality of different aminoalcohol lipidoid compounds with various numbers of tails. Certain amines may be fully functionalized with two epoxide-derived compound tails while other molecules will not be completely functionalized with epoxide-derived compound tails. For example, a diamine or polyamine may include one, two, three, or four epoxide-derived compound tails off the various amino moieties of the molecule resulting in primary, secondary, and tertiary amines. In certain embodiments, all the amino groups are not fully functionalized. In certain embodiments, two of the same types of epoxide-terminated compounds are used. In other embodiments, two or more different epoxide-terminated compounds are used. The synthesis of the aminoalcohol lipidoid compounds is performed with or without solvent, and the synthesis may be performed at higher temperatures ranging from 30-100° C., preferably at approximately 50-90° C. The prepared aminoalcohol lipidoid compounds may be optionally purified. For example, the mixture of aminoalcohol lipidoid compounds may be purified to yield an aminoalcohol lipidoid compound with a particular number of epoxide-derived compound tails. Or the mixture may be purified to yield a particular stereo- or regioisomer. The aminoalcohol lipidoid compounds may also be alkylated using an alkyl halide (e.g., methyl iodide) or other alkylating agent, and/or they may be acylated.

US Patent Publication No. 20110293703 also provides libraries of aminoalcohol lipidoid compounds prepared by the inventive methods. These aminoalcohol lipidoid compounds may be prepared and/or screened using high-throughput techniques involving liquid handlers, robots, microtiter plates, computers, etc. In certain embodiments, the aminoalcohol lipidoid compounds are screened for their ability to transfect polynucleotides or other agents (e.g., proteins, peptides, small molecules) into the cell.

US Patent Publication No. 20130302401 relates to a class of poly(beta-amino alcohols) (PBAAs) has been prepared using combinatorial polymerization. The inventive PBAAs may be used in biotechnology and biomedical applications as coatings (such as coatings of films or multilayer films for medical devices or implants), additives, materials, excipients, non-biofouling agents, micropatterning agents, and cellular encapsulation agents. When used as surface coatings, these PBAAs elicited different levels of inflammation, both in vitro and in vivo, depending on their chemical structures. The large chemical diversity of this class of materials allowed us to identify polymer coatings that inhibit macrophage activation in vitro. Furthermore, these coatings reduce the recruitment of inflammatory cells, and reduce fibrosis, following the subcutaneous implantation of carboxylated polystyrene microparticles. These polymers may be used to form polyelectrolyte complex capsules for cell encapsulation. The invention may also have many other biological applications such as antimicrobial coatings, DNA or siRNA delivery, and stem cell tissue engineering. The teachings of US Patent Publication No. 20130302401 may be applied to the system of the present invention.

In another embodiment, lipid nanoparticles (LNPs) are contemplated. An antitransthyretin small interfering RNA has been encapsulated in lipid nanoparticles and delivered to humans (see, e.g., Coelho et al., N Engl J Med 2013; 369:819-29), and such a system may be adapted and applied to the CRISPR Cas system of the present invention. Doses of about 0.01 to about 1 mg per kg of body weight administered intravenously are contemplated. Medications to reduce the risk of infusion-related reactions are contemplated, such as dexamethasone, acetampinophen, diphenhydramine or cetirizine, and ranitidine are contemplated. Multiple doses of about 0.3 mg per kilogram every 4 weeks for five doses are also contemplated.

Zhu et al. (US20140348900) provides for a process for preparing liposomes, lipid discs, and other lipid nanoparticles using a multi-port manifold, wherein the lipid solution stream, containing an organic solvent, is mixed with two or more streams of aqueous solution (e.g., buffer). In some aspects, at least some of the streams of the lipid and aqueous solutions are not directly opposite of each other. Thus, the process does not require dilution of the organic solvent as an additional step. In some embodiments, one of the solutions may also contain an active pharmaceutical ingredient (API). This invention provides a robust process of liposome manufacturing with different lipid formulations and different payloads. Particle size, morphology, and the manufacturing scale can be controlled by altering the port size and number of the manifold ports, and by selecting the flow rate or flow velocity of the lipid and aqueous solutions.

Cullis et al. (US 20140328759) provides limit size lipid nanoparticles with a diameter from 10-100 nm, in particular comprising a lipid bilayer surrounding an aqueous core. Methods and apparatus for preparing such limit size lipid nanoparticles are also disclosed. Manoharan et al. (US 20140308304) provides cationic lipids of formula (I) that can be utilized for delivery. The cationic lipid can be used with other lipid components such as cholesterol and PEG-lipids to form lipid nanoparticles with oligonucleotides, to facilitate the cellular uptake and endosomal escape, and to knockdown target mRNA both in vitro and in vivo.

LNPs have been shown to be highly effective in delivering siRNAs to the liver (see, e.g., Tabernero et al., Cancer Discovery, April 2013, Vol. 3, No. 4, pages 363-470) and are therefore contemplated for delivering RNA encoding CRISPR Cas to the liver. A dosage of about four doses of 6 mg/kg of the LNP every two weeks may be contemplated. Tabernero et al. demonstrated that tumor regression was observed after the first 2 cycles of LNPs dosed at 0.7 mg/kg, and by the end of 6 cycles the patient had achieved a partial response with complete regression of the lymph node metastasis and substantial shrinkage of the liver tumors. A complete response was obtained after 40 doses in this patient, who has remained in remission and completed treatment after receiving doses over 26 months. Two patients with RCC and extrahepatic sites of disease including kidney, lung, and lymph nodes that were progressing following prior therapy with VEGF pathway inhibitors had stable disease at all sites for approximately 8 to 12 months, and a patient with PNET and liver metastases continued on the extension study for 18 months (36 doses) with stable disease.

However, the charge of the LNP must be taken into consideration. As cationic lipids combined with negatively charged lipids to induce nonbilayer structures that facilitate intracellular delivery. Because charged LNPs are rapidly cleared from circulation following intravenous injection, ionizable cationic lipids with pKa values below 7 were developed (see, e.g., Rosin et al, Molecular Therapy, vol. 19, no. 12, pages 1286-2200, December 2011). Negatively charged polymers such as RNA may be loaded into LNPs at low pH values (e.g., pH 4) where the ionizable lipids display a positive charge. However, at physiological pH values, the LNPs exhibit a low surface charge compatible with longer circulation times. Four species of ionizable cationic lipids have been focused upon, namely 1,2-dilineoyl-3-dimethylammonium-propane (DLinDAP), 1,2-dilinoleyloxy-3-N,N-dimethylaminopropane (DLinDMA), 1,2-dilinoleyloxy-keto-N,N-dimethyl-3-aminopropane (DLinKDMA), and 1,2-dilinoleyl-4-(2-dimethylaminoethyl)-[1,3]-dioxolane (DLinKC2-DMA). It has been shown that LNP siRNA systems containing these lipids exhibit remarkably different gene silencing properties in hepatocytes in vivo, with potencies varying according to the series DLinKC2-DMA>DLinKDMA>DLinDMA>>DLinDAP employing a Factor VII gene silencing model (see, e.g., Rosin et al, Molecular Therapy, vol. 19, no. 12, pages 1286-2200, December 2011). A dosage of 1 μg/ml of LNP or CRISPR-Cas RNA in or associated with the LNP may be contemplated, especially for a formulation containing DLinKC2-DMA.

Preparation of LNPs and CRISPR Cas encapsulation may be used/and or adapted from Rosin et al, Molecular Therapy, vol. 19, no. 12, pages 1286-2200, December 2011). The cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)-[1,3]-dioxolane (DLinKC2-DMA), (3-o-[2″-(methoxypolyethyleneglycol 2000) succinoyl]-1,2-dimyristoyl-sn-glycol (PEG-S-DMG), and R-3-[(ω-methoxy-poly(ethylene glycol)2000) carbamoyl]-1,2-dimyristyloxlpropyl-3-amine (PEG-C-DOMG) may be provided by Tekmira Pharmaceuticals (Vancouver, Canada) or synthesized. Cholesterol may be purchased from Sigma (St Louis, Mo.). The specific CRISPR Cas RNA may be encapsulated in LNPs containing DLinDAP, DLinDMA, DLinK-DMA, and DLinKC2-DMA (cationic lipid:DSPC:CHOL: PEGS-DMG or PEG-C-DOMG at 40:10:40:10 molar ratios). When required, 0.2% SP-DiOC18 (Invitrogen, Burlington, Canada) may be incorporated to assess cellular uptake, intracellular delivery, and biodistribution. Encapsulation may be performed by dissolving lipid mixtures comprised of cationic lipid:DSPC:cholesterol:PEG-c-DOMG (40:10:40:10 molar ratio) in ethanol to a final lipid concentration of 10 mmol/l. This ethanol solution of lipid may be added drop-wise to 50 mmol/l citrate, pH 4.0 to form multilamellar vesicles to produce a final concentration of 30% ethanol vol/vol. Large unilamellar vesicles may be formed following extrusion of multilamellar vesicles through two stacked 80 nm Nuclepore polycarbonate filters using the Extruder (Northern Lipids, Vancouver, Canada). Encapsulation may be achieved by adding RNA dissolved at 2 mg/ml in 50 mmol/l citrate, pH 4.0 containing 30% ethanol vol/vol drop-wise to extruded preformed large unilamellar vesicles and incubation at 31° C. for 30 minutes with constant mixing to a final RNA/lipid weight ratio of 0.06/1 wt/wt. Removal of ethanol and neutralization of formulation buffer were performed by dialysis against phosphate-buffered saline (PBS), pH 7.4 for 16 hours using Spectra/Por 2 regenerated cellulose dialysis membranes. Nanoparticle size distribution may be determined by dynamic light scattering using a NICOMP 370 particle sizer, the vesicle/intensity modes, and Gaussian fitting (Nicomp Particle Sizing, Santa Barbara, Calif.). The particle size for all three LNP systems may be ˜70 nm in diameter. RNA encapsulation efficiency may be determined by removal of free RNA using VivaPureD MiniH columns (Sartorius Stedim Biotech) from samples collected before and after dialysis. The encapsulated RNA may be extracted from the eluted nanoparticles and quantified at 260 nm. RNA to lipid ratio was determined by measurement of cholesterol content in vesicles using the Cholesterol E enzymatic assay from Wako Chemicals USA (Richmond, Va.). In conjunction with the herein discussion of LNPs and PEG lipids, PEGylated liposomes or LNPs are likewise suitable for delivery of a CRISPR-Cas system or components thereof.

Preparation of large LNPs may be used/and or adapted from Rosin et al, Molecular Therapy, vol. 19, no. 12, pages 1286-2200, December 2011. A lipid premix solution (20.4 mg/ml total lipid concentration) may be prepared in ethanol containing DLinKC2-DMA, DSPC, and cholesterol at 50:10:38.5 molar ratios. Sodium acetate may be added to the lipid premix at a molar ratio of 0.75:1 (sodium acetate:DLinKC2-DMA). The lipids may be subsequently hydrated by combining the mixture with 1.85 volumes of citrate buffer (10 mmol/l, pH 3.0) with vigorous stirring, resulting in spontaneous liposome formation in aqueous buffer containing 35% ethanol. The liposome solution may be incubated at 37° C. to allow for time-dependent increase in particle size. Aliquots may be removed at various times during incubation to investigate changes in liposome size by dynamic light scattering (Zetasizer Nano ZS, Malvern Instruments, Worcestershire, UK). Once the desired particle size is achieved, an aqueous PEG lipid solution (stock=10 mg/ml PEG-DMG in 35% (vol/vol) ethanol) may be added to the liposome mixture to yield a final PEG molar concentration of 3.5% of total lipid. Upon addition of PEG-lipids, the liposomes should their size, effectively quenching further growth. RNA may then be added to the empty liposomes at an RNA to total lipid ratio of approximately 1:10 (wt:wt), followed by incubation for 30 minutes at 37° C. to form loaded LNPs. The mixture may be subsequently dialyzed overnight in PBS and filtered with a 0.45-μm syringe filter.

### Liposomes

In one aspect, the invention provides a particle delivery system comprising a composite virus particle, wherein the composite virus particle comprises a lipid, a virus capsid protein, and a protein or peptide. The peptide or protein can be up to one megadalton in size.

In one embodiment, the particle delivery system comprises a virus particle adsorbed to a liposome. In one embodiment, the liposome comprises a cationic lipid.

In one embodiment, the liposome of the particle delivery system comprises the CRISPR-Cas system component.

In one aspect, the invention provides a delivery system comprising one or more hybrid virus capsid proteins in combination with a lipid particle, wherein the hybrid virus capsid protein comprises at least a portion of a virus capsid protein attached to at least a portion of a non-capsid protein.

In one embodiment, the virus capsid protein of the delivery system is attached to the surface of the lipid particle. In one embodiment, the virus capsid protein is attached to the surface of the lipid particle by an electrostatic interaction or by hydrophobic interaction.

In one embodiment, the lipid particle has a diameter of 50-1000 nm, preferably 100-1000 nm.

In one embodiment, the delivery system comprises a protein or peptide, wherein the protein or peptide has a molecular weight of up to a megadalton. In one embodiment, the protein or peptide has a molecular weight in the range of 110 to 160 kDa.

In one embodiment, the delivery system comprises a protein or peptide, wherein the protein or peptide comprises a nucleic acid modifying protein or peptide. In one embodiment, the protein or peptide comprises one or more domains of a Cas9, a Cpf1 or a C2c2.

In one embodiment, the lipid, lipid particle or lipid layer of the delivery system comprises at least one cationic lipid.

In one embodiment, the lipid compound is preferably a bio-reducible material, e.g., a bio-reducible polymer and a bio-reducible lipid-like compound.

In one aspect, the lipid or lipid-like compound comprises a hydrophilic head, a hydrophobic tail, and a linker.

In one embodiment, the hydrophilic head contains one or more hydrophilic functional groups, e.g., hydroxyl, carboxyl, amino, sulfhydryl, phosphate, amide, ester, ether, carbamate, carbonate, carbamide and phosphodiester. These groups can form hydrogen bonds and are optionally positively or negatively charged.

In one embodiment, the hydrophobic tail is a saturated or unsaturated, linear or branched, acyclic or cyclic, aromatic or nonaromatic hydrocarbon moiety containing a disulfide bond and 8-24 carbon atoms. One or more of the carbon atoms can be replaced with a heteroatom, such as N, O, P, B, S, Si, Sb, Al, Sn, As, Se, and Ge. The lipid or lipid-like compounds containing disulfide bond can be bioreducible.

In one embodiment, the linker of the lipid or lipid-like compound links the hydrophilic head and the hydrophobic tail. The linker can be any chemical group that is hydrophilic or hydrophobic, polar or non-polar, e.g., O, S, Si, amino, alkylene, ester, amide, carbamate, carbamide, carbonate phosphate, phosphite, sulfate, sulfite, and thiosulfate.

The lipid or lipid-like compounds described above include the compounds themselves, as well as their salts and solvates, if applicable. A salt, for example, can be formed between an anion and a positively charged group (e.g., amino) on a lipid-like compound. Suitable anions include chloride, bromide, iodide, sulfate, nitrate, phosphate, citrate, methanesulfonate, trifluoroacetate, acetate, malate, tosylate, tartrate, fumurate, glutamate, glucuronate, lactate, glutarate, and maleate. Likewise, a salt can also be formed between a cation and a negatively charged group (e.g., carboxylate) on a lipid-like compound. Suitable cations include sodium ion, potassium ion, magnesium ion, calcium ion, and an ammonium cation such as tetramethylammonium ion. The lipid-like compounds also include those salts containing quaternary nitrogen atoms. A solvate refers to a complex formed between a lipid-like compound and a pharmaceutically acceptable solvent. Examples of pharmaceutically acceptable solvents include water, ethanol, isopropanol, ethyl acetate, acetic acid, and ethanolamine.

Delivery or administration according to the invention can be performed with liposomes. Liposomes are spherical vesicle structures composed of a uni- or multilamellar lipid bilayer surrounding internal aqueous compartments and a relatively impermeable outer lipophilic phospholipid bilayer. Liposomes have gained considerable attention as drug delivery carriers because they are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

Liposomes can be made from several different types of lipids; however, phospholipids are most commonly used to generate liposomes as drug carriers. Although liposome formation is spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review).

Several other additives may be added to liposomes in order to modify their structure and properties. For instance, either cholesterol or sphingomyelin may be added to the liposomal mixture in order to help stabilize the liposomal structure and to prevent the leakage of the liposomal inner cargo. Further, liposomes are prepared from hydrogenated egg phosphatidylcholine or egg phosphatidylcholine, cholesterol, and dicetyl phosphate, and their mean vesicle sizes were adjusted to about 50 and 100 nm. (see, e.g., Spuch and Navarro, Journal of Drug Delivery, vol. 2011, Article ID 469679, 12 pages, 2011. doi:10.1155/2011/469679 for review).

Additional formulations, including liposome formulation, lipid particles and other lipids, such as cationic lipids for use in delivery systems can be as disclosed in International Patent Publication WO 2019/135816 at [0710]-[0727], incorporated herein by reference. Similarly, supercharged proteins, a class of engineered or naturally occurring proteins with unusually high positive or negative net theoretical charge that may be employed in delivery of CRISPR Cas system(s) or component(s) thereof or nucleic acid molecule(s) coding therefor; and cell penetrating peptides for delivery of CRISPR Cas systems can be as described in International Patent Publication WO 2019/135816 at [0728]-[0739], incorporated herein by reference.

### Targeting

In further embodiments, the system can comprise a delivery enhancer. The delivery enhancer can be a cellular permeability enhancer.

PNAs will act as high-fidelity DNA readers as well as a scaffold for display of synthetic nucleases, with reduced size compared to that of Cas protein-guide RNA complex. Advantageously, the size reduction can allow delivery of multiple editors into a cell type of interest and may even allow highly multiplexed editing, with cellular permeability and other deliver enhancers enhancing the novel platform to allow multiplexed precision genome editing on an unprecedented scale.

### Delivery

In some embodiments delivery methods include: Cationic Lipid Mediated “direct” delivery of Enzyme-Guide complex (RiboNucleoProtein) and electroporation of plasmid DNA.

With regard to targeting moieties, mention is made of Deshpande et al, “Current trends in the use of liposomes for tumor targeting,” Nanomedicine (Lond). 8(9), doi:10.2217/nnm.13.118 (2013), and the documents it cites, all of which are incorporated herein by reference. Mention is also made of WO/2016/027264, and the documents it cites, all of which are incorporated herein by reference. And mention is made of Lorenzer et al, “Going beyond the liver: Progress and challenges of targeted delivery of siRNA therapeutics,” Journal of Controlled Release, 203: 1-15 (2015), and the documents it cites, all of which are incorporated herein by reference.

An actively targeting lipid particle or nanoparticle or liposome or lipid bilayer delivery system (generally as to embodiments of the invention, “lipid entity of the invention” delivery systems) are prepared by conjugating targeting moieties, including small molecule ligands, peptides and monoclonal antibodies, on the lipid or liposomal surface; for example, certain receptors, such as folate and transferrin (Tf) receptors (TfR), are overexpressed on many cancer cells and have been used to make liposomes tumor cell specific. Liposomes that accumulate in the tumor microenvironment can be subsequently endocytosed into the cells by interacting with specific cell surface receptors. To efficiently target liposomes to cells, such as cancer cells, it is useful that the targeting moiety have an affinity for a cell surface receptor and to link the targeting moiety in sufficient quantities to have optimum affinity for the cell surface receptors; and determining these aspects are within the ambit of the skilled artisan. In the field of active targeting, there are a number of cell-, e.g., tumor-, specific targeting ligands.

Also as to active targeting, with regard to targeting cell surface receptors such as cancer cell surface receptors, targeting ligands on liposomes can provide attachment of liposomes to cells, e.g., vascular cells, via a nonintemalizing epitope; and, this can increase the extracellular concentration of that which is being delivered, thereby increasing the amount delivered to the target cells. A strategy to target cell surface receptors, such as cell surface receptors on cancer cells, such as overexpressed cell surface receptors on cancer cells, is to use receptor-specific ligands or antibodies. Many cancer cell types display upregulation of tumor-specific receptors. For example, TfRs and folate receptors (FRs) are greatly overexpressed by many tumor cell types in response to their increased metabolic demand. Folic acid can be used as a targeting ligand for specialized delivery owing to its ease of conjugation to nanocarriers, its high affinity for FRs and the relatively low frequency of FRs, in normal tissues as compared with their overexpression in activated macrophages and cancer cells, e.g., certain ovarian, breast, lung, colon, kidney and brain tumors. Overexpression of FR on macrophages is an indication of inflammatory diseases, such as psoriasis, Crohn's disease, rheumatoid arthritis and atherosclerosis; accordingly, folate-mediated targeting of the invention can also be used for studying, addressing or treating inflammatory disorders, as well as cancers. Folate-linked lipid particles or nanoparticles or liposomes or lipid by layers of the invention (“lipid entity of the invention”) deliver their cargo intracellularly through receptor-mediated endocytosis. Intracellular trafficking can be directed to acidic compartments that facilitate cargo release, and, most importantly, release of the cargo can be altered or delayed until it reaches the cytoplasm or vicinity of target organelles. Delivery of cargo using a lipid entity of the invention having a targeting moiety, such as a folate-linked lipid entity of the invention, can be superior to nontargeted lipid entity of the invention. The attachment of folate directly to the lipid head groups may not be favorable for intracellular delivery of folate-conjugated lipid entity of the invention, since they may not bind as efficiently to cells as folate attached to the lipid entity of the invention surface by a spacer, which may can enter cancer cells more efficiently. A lipid entity of the invention coupled to folate can be used for the delivery of complexes of lipid, e.g., liposome, e.g., anionic liposome and virus or capsid or envelope or virus outer protein, such as those herein discussed such as adenovirous or AAV. Tf is a monomeric serum glycoprotein of approximately 80 KDa involved in the transport of iron throughout the body. Tf binds to the TfR and translocates into cells via receptor-mediated endocytosis. The expression of TfR is can be higher in certain cells, such as tumor cells (as compared with normal cells and is associated with the increased iron demand in rapidly proliferating cancer cells. Accordingly, the invention comprehends a TfR-targeted lipid entity of the invention, e.g., as to liver cells, liver cancer, breast cells such as breast cancer cells, colon such as colon cancer cells, ovarian cells such as ovarian cancer cells, head, neck and lung cells, such as head, neck and non-small-cell lung cancer cells, cells of the mouth such as oral tumor cells.

Also as to active targeting, a lipid entity of the invention can be multifunctional, i.e., employ more than one targeting moiety such as CPP, along with Tf; a bifunctional system; e.g., a combination of Tf and poly-L-arginine which can provide transport across the endothelium of the blood-brain barrier. EGFR, is a tyrosine kinase receptor belonging to the ErbB family of receptors that mediates cell growth, differentiation and repair in cells, especially non-cancerous cells, but EGF is overexpressed in certain cells such as many solid tumors, including colorectal, non-small-cell lung cancer, squamous cell carcinoma of the ovary, kidney, head, pancreas, neck and prostate, and especially breast cancer. The invention comprehends EGFR-targeted monoclonal antibody(ies) linked to a lipid entity of the invention. HER-2 is often overexpressed in patients with breast cancer, and is also associated with lung, bladder, prostate, brain and stomach cancers. HER-2, encoded by the ERBB2 gene. The invention comprehends a HER-2-targeting lipid entity of the invention, e.g., an anti-HER-2-antibody (or binding fragment thereof)-lipid entity of the invention, a HER-2-targeting-PEGylated lipid entity of the invention (e.g., having an anti-HER-2-antibody or binding fragment thereof), a HER-2-targeting-maleimide-PEG polymer-lipid entity of the invention (e.g., having an anti-HER-2-antibody or binding fragment thereof). Upon cellular association, the receptor-antibody complex can be internalized by formation of an endosome for delivery to the cytoplasm. With respect to receptor-mediated targeting, the skilled artisan takes into consideration ligand/target affinity and the quantity of receptors on the cell surface, and that PEGylation can act as a barrier against interaction with receptors. The use of antibody-lipid entity of the invention targeting can be advantageous. Multivalent presentation of targeting moieties can also increase the uptake and signaling properties of antibody fragments. In practice of the invention, the skilled person takes into account ligand density (e.g., high ligand densities on a lipid entity of the invention may be advantageous for increased binding to target cells). Preventing early by macrophages can be addressed with a sterically stabilized lipid entity of the invention and linking ligands to the terminus of molecules such as PEG, which is anchored in the lipid entity of the invention (e.g., lipid particle or nanoparticle or liposome or lipid bilayer). The microenvironment of a cell mass such as a tumor microenvironment can be targeted; for instance, it may be advantageous to target cell mass vasculature, such as the tumor vasculature microenvironment. Thus, the invention comprehends targeting VEGF. VEGF and its receptors are well-known proangiogenic molecules and are well-characterized targets for antiangiogenic therapy. Many small-molecule inhibitors of receptor tyrosine kinases, such as VEGFRs or basic FGFRs, have been developed as anticancer agents and the invention comprehends coupling any one or more of these peptides to a lipid entity of the invention, e.g., phage IVO peptide(s) (e.g., via or with a PEG terminus), tumor-homing peptide APRPG such as APRPG-PEG-modified. VCAM, the vascular endothelium plays a key role in the pathogenesis of inflammation, thrombosis and atherosclerosis. CAMs are involved in inflammatory disorders, including cancer, and are a logical target, E- and P-selectins, VCAM-1 and ICAMs. Can be used to target a lipid entity of the invention, e.g., with PEGylation. Matrix metalloproteases (MMPs) belong to the family of zinc-dependent endopeptidases. They are involved in tissue remodeling, tumor invasiveness, resistance to apoptosis and metastasis. There are four MMP inhibitors called TIMP1-4, which determine the balance between tumor growth inhibition and metastasis; a protein involved in the angiogenesis of tumor vessels is MT1-MMP, expressed on newly formed vessels and tumor tissues. The proteolytic activity of MT1-MMP cleaves proteins, such as fibronectin, elastin, collagen and laminin, at the plasma membrane and activates soluble MMPs, such as MMP-2, which degrades the matrix. An antibody or fragment thereof such as a Fab′ fragment can be used in the practice of the invention such as for an antihuman MT1-MMP monoclonal antibody linked to a lipid entity of the invention, e.g., via a spacer such as a PEG spacer. αβ-integrins or integrins are a group of transmembrane glycoprotein receptors that mediate attachment between a cell and its surrounding tissues or extracellular matrix. Integrins contain two distinct chains (heterodimers) called α- and β-subunits. The tumor tissue-specific expression of integrin receptors can be been utilized for targeted delivery in the invention, e.g., whereby the targeting moiety can be an RGD peptide such as a cyclic RGD. Aptamers are ssDNA or RNA oligonucleotides that impart high affinity and specific recognition of the target molecules by electrostatic interactions, hydrogen bonding and hydro phobic interactions as opposed to the Watson-Crick base pairing, which is typical for the bonding interactions of oligonucleotides. Aptamers as a targeting moiety can have advantages over antibodies: aptamers can demonstrate higher target antigen recognition as compared with antibodies; aptamers can be more stable and smaller in size as compared with antibodies; aptamers can be easily synthesized and chemically modified for molecular conjugation; and aptamers can be changed in sequence for improved selectivity and can be developed to recognize poorly immunogenic targets. Such moieties as a sgc8 aptamer can be used as a targeting moiety (e.g., via covalent linking to the lipid entity of the invention, e.g., via a spacer, such as a PEG spacer). The targeting moiety can be stimuli-sensitive, e.g., sensitive to an externally applied stimuli, such as magnetic fields, ultrasound or light; and pH-triggering can also be used, e.g., a labile linkage can be used between a hydrophilic moiety such as PEG and a hydrophobic moiety such as a lipid entity of the invention, which is cleaved only upon exposure to the relatively acidic conditions characteristic of the a particular environment or microenvironment such as an endocytic vacuole or the acidotic tumor mass. pH-sensitive copolymers can also be incorporated in embodiments of the invention can provide shielding; diortho esters, vinyl esters, cysteine-cleavable lipopolymers, double esters and hydrazones are a few examples of pH-sensitive bonds that are quite stable at pH 7.5, but are hydrolyzed relatively rapidly at pH 6 and below, e.g., a terminally alkylated copolymer of N-isopropylacrylamide and methacrylic acid that copolymer facilitates destabilization of a lipid entity of the invention and release in compartments with decreased pH value; or, the invention comprehends ionic polymers for generation of a pH-responsive lipid entity of the invention (e.g., poly(methacrylic acid), poly(diethylaminoethyl methacrylate), poly(acrylamide) and poly(acrylic acid)). Temperature-triggered delivery is also within the ambit of the invention. Many pathological areas, such as inflamed tissues and tumors, show a distinctive hyperthermia compared with normal tissues. Utilizing this hyperthermia is an attractive strategy in cancer therapy since hyperthermia is associated with increased tumor permeability and enhanced uptake. This technique involves local heating of the site to increase microvascular pore size and blood flow, which, in turn, can result in an increased extravasation of embodiments of the invention. Temperature-sensitive lipid entity of the invention can be prepared from thermosensitive lipids or polymers with a low critical solution temperature. Above the low critical solution temperature (e.g., at site such as tumor site or inflamed tissue site), the polymer precipitates, disrupting the liposomes to release. Lipids with a specific gel-to-liquid phase transition temperature are used to prepare these lipid entities of the invention; and a lipid for a thermosensitive embodiment can be dipalmitoylphosphatidylcholine. Thermosensitive polymers can also facilitate destabilization followed by release, and a useful thermosensitive polymer is poly (N-isopropylacrylamide). Another temperature triggered system can employ lysolipid temperature-sensitive liposomes. The invention also comprehends redox-triggered delivery: The difference in redox potential between normal and inflamed or tumor tissues, and between the intra- and extra-cellular environments has been exploited for delivery; e.g., GSH is a reducing agent abundant in cells, especially in the cytosol, mitochondria and nucleus. The GSH concentrations in blood and extracellular matrix are just one out of 100 to one out of 1000 of the intracellular concentration, respectively. This high redox potential difference caused by GSH, cysteine and other reducing agents can break the reducible bonds, destabilize a lipid entity of the invention and result in release of payload. The disulfide bond can be used as the cleavable/reversible linker in a lipid entity of the invention, because it causes sensitivity to redox owing to the disulfideto-thiol reduction reaction; a lipid entity of the invention can be made reduction sensitive by using two (e.g., two forms of a disulfide-conjugated multifunctional lipid as cleavage of the disulfide bond (e.g., via tris(2-carboxyethyl)phosphine, dithiothreitol, L-cysteine or GSH), can cause removal of the hydrophilic head group of the conjugate and alter the membrane organization leading to release of payload. Calcein release from reduction-sensitive lipid entity of the invention containing a disulfide conjugate can be more useful than a reduction-insensitive embodiment. Enzymes can also be used as a trigger to release payload. Enzymes, including MMPs (e.g. MMP2), phospholipase A2, alkaline phosphatase, transglutaminase or phosphatidylinositol-specific phospholipase C, have been found to be overexpressed in certain tissues, e.g., tumor tissues. In the presence of these enzymes, specially engineered enzyme-sensitive lipid entity of the invention can be disrupted and release the payload. an MMP2-cleavable octapeptide (Gly-Pro-Leu-Gly-Ile-Ala-Gly-Gln) can be incorporated into a linker, and can have antibody targeting, e.g., antibody 2C5. The invention also comprehends light- or energy-triggered delivery, e.g., the lipid entity of the invention can be light-sensitive, such that light or energy can facilitate structural and conformational changes, which lead to direct interaction of the lipid entity of the invention with the target cells via membrane fusion, photo-isomerism, photofragmentation or photopolymerization; such a moiety therefor can be benzoporphyrin photosensitizer. Ultrasound can be a form of energy to trigger delivery; a lipid entity of the invention with a small quantity of particular gas, including air or perfluorated hydrocarbon can be triggered to release with ultrasound, e.g., low-frequency ultrasound (LFUS). Magnetic delivery: A lipid entity of the invention can be magnetized by incorporation of magnetites, such as Fe3O4 or γ-Fe2O3, e.g., those that are less than 10 nm in size. Targeted delivery can be then by exposure to a magnetic field.

Also as to active targeting, the invention also comprehends intracellular delivery. Since liposomes follow the endocytic pathway, they are entrapped in the endosomes (pH 6.5-6) and subsequently fuse with lysosomes (pH<5), where they undergo degradation that results in a lower therapeutic potential. The low endosomal pH can be taken advantage of to escape degradation. Fusogenic lipids or peptides, which destabilize the endosomal membrane after the conformational transition/activation at a lowered pH. Amines are protonated at an acidic pH and cause endosomal swelling and rupture by a buffer effect Unsaturated dioleoylphosphatidylethanolamine (DOPE) readily adopts an inverted hexagonal shape at a low pH, which causes fusion of liposomes to the endosomal membrane. This process destabilizes a lipid entity containing DOPE and releases the cargo into the cytoplasm; fusogenic lipid GALA, cholesteryl-GALA and PEG-GALA may show a highly efficient endosomal release; a pore-forming protein listeriolysin O may provide an endosomal escape mechanism; and, histidine-rich peptides have the ability to fuse with the endosomal membrane, resulting in pore formation, and can buffer the proton pump causing membrane lysis.

Also as to active targeting, cell-penetrating peptides (CPPs) facilitate uptake of macromolecules through cellular membranes and, thus, enhance the delivery of CPP-modified molecules inside the cell. CPPs can be split into two classes: amphipathic helical peptides, such as transportan and MAP, where lysine residues are major contributors to the positive charge; and Arg-rich peptides, such as TATp, Antennapedia or penetratin. TATp is a transcription-activating factor with 86 amino acids that contains a highly basic (two Lys and six Arg among nine residues) protein transduction domain, which brings about nuclear localization and RNA binding. Other CPPs that have been used for the modification of liposomes include the following: the minimal protein transduction domain of Antennapedia, a Drosophilia homeoprotein, called penetratin, which is a 16-mer peptide (residues 43-58) present in the third helix of the homeodomain; a 27-amino acid-long chimeric CPP, containing the peptide sequence from the amino terminus of the neuropeptide galanin bound via the Lys residue, mastoparan, a wasp venom peptide; VP22, a major structural component of HSV-1 facilitating intracellular transport and transportan (18-mer) amphipathic model peptide that translocates plasma membranes of mast cells and endothelial cells by both energy-dependent and -independent mechanisms. The invention comprehends a lipid entity of the invention modified with CPP(s), for intracellular delivery that may proceed via energy dependent macropinocytosis followed by endosomal escape. The invention further comprehends organelle-specific targeting. A lipid entity of the invention surface-functionalized with the triphenylphosphonium (TPP) moiety or a lipid entity of the invention with a lipophilic cation, rhodamine 123 can be effective in delivery of cargo to mitochondria. DOPE/sphingomyelin/stearyl-octa-arginine can delivers cargos to the mitochondrial interior via membrane fusion. A lipid entity of the invention surface modified with a lysosomotropic ligand, octadecyl rhodamine B can deliver cargo to lysosomes. Ceramides are useful in inducing lysosomal membrane permeabilization; the invention comprehends intracellular delivery of a lipid entity of the invention having a ceramide. The invention further comprehends a lipid entity of the invention targeting the nucleus, e.g., via a DNA-intercalating moiety. The invention also comprehends multifunctional liposomes for targeting, i.e., attaching more than one functional group to the surface of the lipid entity of the invention, for instance to enhances accumulation in a desired site and/or promotes organelle-specific delivery and/or target a particular type of cell and/or respond to the local stimuli such as temperature (e.g., elevated), pH (e.g., decreased), respond to externally applied stimuli such as a magnetic field, light, energy, heat or ultrasound and/or promote intracellular delivery of the cargo. All of these are considered actively targeting moieties.

An embodiment of the invention includes the particle delivery system comprising an actively targeting lipid particle or nanoparticle or liposome or lipid iylayer delivery system; or comprising a lipid particle or nanoparticle or liposome or lipid bilayer comprising a targeting moiety whereby there is active targeting or wherein the targeting moiety is an actively targeting moiety. A targeting moiety can be one or more targeting moieties, and a targeting moiety can be for any desired type of targeting such as, e.g., to target a cell such as any herein-mentioned; or to target an organelle such as any herein-mentioned; or for targeting a response such as to a physical condition such as heat, energy, ultrasound, light, pH, chemical such as enzymatic, or magnetic stimuli; or to target to achieve a particular outcome such as delivery of payload to a particular location, such as by cell penetration.

It should be understood that as to each possible targeting or active targeting moiety herein-discussed, there is an aspect of the invention wherein the delivery system comprises such a targeting or active targeting moiety. Likewise, the following table provides exemplary targeting moieties that can be used in the practice of the invention an as to each an aspect of the invention provides a delivery system that comprises such a targeting moiety.

TABLE 1

| Targeting Moiety | Target Molecule | Target Cell or Tissue |
|---|---|---|
| folate | folate receptor | cancer cells |
| transferrin | transferrin receptor | cancer cells |
| Antibody CC52 | rat CC531 | rat colon |
| | | adenocarcinoma CC531 |
| anti-HER2 | HER2 | HER2-overexpressing |
| antibody | | tumors |
| anti-GD2 | GD2 | neuroblastoma, melanoma |
| anti-EGFR | EGFR | tumor cells overexpressing |
| | | EGFR |
| pH-dependent | | ovarian carcinoma |
| fusogenic | | |
| peptide diINF-7 | | |
| anti-VEGFR | VEGF Receptor | tumor vasculature |
| anti-CD19 | CD19 | leukemia, lymphoma |
| | (B cell marker) | |
| cell-penetrating | | blood-brain barrier |
| peptide | | |
| cyclic arginine- | avβ3 | glioblastoma cells, |
| glycine-aspartic | | human umbilical vein |
| acid-tyrosine- | | endothelial cells, |
| cysteine peptide | | tumor angiogenesis |
| (c(RGDyC)-LP) | | |
| ASSHN peptide | | endothelial |
| | | progenitor cells; |
| | | anti-cancer |
| PR_b peptide | α₅β₁ integrin | cancer cells |
| AG86 peptide | α₆β₄ integrin | cancer cells |
| KCCYSL | HER-2 receptor | cancer cells |
| (P6.1 peptide) | | |
| affinity peptide LN | Aminopeptidase N | APN-positive tumor |
| (YEVGHRC) | (APN/CD13) | |
| synthetic | Somatostatin | breast cancer |
| somatostatin | receptor 2 | |
| analogue | (SSTR2) | |
| anti-CD20 | B-lymphocytes | B cell lymphoma |
| monoclonal | | |
| antibody | | |

Thus, in an embodiment of the particle delivery system, the targeting moiety comprises a receptor ligand, such as, for example, hyaluronic acid for CD44 receptor, galactose for hepatocytes, or antibody or fragment thereof such as a binding antibody fragment against a desired surface receptor, and as to each of a targeting moiety comprising a receptor ligand, or an antibody or fragment thereof such as a binding fragment thereof, such as against a desired surface receptor, there is an aspect of the invention wherein the delivery system comprises a targeting moiety comprising a receptor ligand, or an antibody or fragment thereof such as a binding fragment thereof, such as against a desired surface receptor, or hyaluronic acid for CD44 receptor, galactose for hepatocytes (see, e.g., Surace et al, “Lipoplexes targeting the CD44 hyaluronic acid receptor for efficient transfection of breast cancer cells,” J. Mol Pharm 6(4):1062-73; doi: 10.1021/mp800215d (2009); Sonoke et al, “Galactose-modified cationic liposomes as a liver-targeting delivery system for small interfering RNA,” Biol Pharm Bull. 34(8):1338-42 (2011); Torchilin, “Antibody-modified liposomes for cancer chemotherapy,” Expert Opin. Drug Deliv. 5 (9), 1003-1025 (2008); Manjappa et al, “Antibody derivatization and conjugation strategies: application in preparation of stealth immunoliposome to target chemotherapeutics to tumor,” J. Control. Release 150 (1), 2-22 (2011); Sofou S “Antibody-targeted liposomes in cancer therapy and imaging,” Expert Opin. Drug Deliv. 5 (2): 189-204 (2008); Gao J et al, “Antibody-targeted immunoliposomes for cancer treatment,” Mini. Rev. Med. Chem. 13(14): 2026-2035 (2013); Molavi et al, “Anti-CD30 antibody conjugated liposomal doxorubicin with significantly improved therapeutic efficacy against anaplastic large cell lymphoma,” Biomaterials 34(34):8718-25 (2013), each of which and the documents cited therein are hereby incorporated herein by reference).

Moreover, in view of the teachings herein the skilled artisan can readily select and apply a desired targeting moiety in the practice of the invention as to a lipid entity of the invention. The invention comprehends an embodiment wherein the delivery system comprises a lipid entity having a targeting moiety.

In an embodiment of the particle delivery system, the protein comprises a nucleic acid modifying protein.

In some embodiments a non-capsid protein or protein that is not a virus outer protein or a virus envelope (sometimes herein shorthanded as “non-capsid protein”), such as a nucleic acid modifying protein, can have one or more functional moiety(ies) thereon, such as a moiety for targeting or locating, such as an NLS or NES, or an activator or repressor.

In an embodiment of the particle delivery system, a nucleic acid modifying protein can comprise a tag.

In an aspect, the invention provides a virus particle comprising a capsid or outer protein having one or more hybrid virus capsid or outer proteins comprising the virus capsid or outer protein attached to at least a portion of a non-capsid protein or a nucleic acid modifying protein.

In an aspect, the invention provides an in vitro method of delivery comprising contacting the particle delivery system with a cell, optionally a eukaryotic cell, whereby there is delivery into the cell of constituents of the delivery system.

In an aspect, the invention provides an in vitro, a research or study method of delivery comprising contacting the particle delivery system with a cell, optionally a eukaryotic cell, whereby there is delivery into the cell of constituents of the delivery system, obtaining data or results from the contacting, and transmitting the data or results.

In an aspect, the invention provides a cell from or of an in vitro method of delivery, wherein the method comprises contacting the particle delivery system with a cell, optionally a eukaryotic cell, whereby there is delivery into the cell of constituents of the delivery system, and optionally obtaining data or results from the contacting, and transmitting the data or results.

In an aspect, the invention provides a cell from or of an in vitro method of delivery, wherein the method comprises contacting the particle delivery system with a cell, optionally a eukaryotic cell, whereby there is delivery into the cell of constituents of the delivery system, and optionally obtaining data or results from the contacting, and transmitting the data or results; and wherein the cell product is altered compared to the cell not contacted with the delivery system, for example altered from that which would have been wild type of the cell but for the contacting.

In an embodiment, the cell product is non-human or animal.

In an aspect, the invention provides use of the particle delivery system or the delivery system or the virus particle of any one of the above embodiment or the cell of any one of the above embodiment in ex vivo or in vivo gene or genome editing; or for use in in vitro, ex vivo or in vivo gene therapy.

### Methods

In one aspect, the invention provides a method for developing a biologically active agent that modulates a cell signaling event associated with a disease gene. In some embodiments, a disease gene is any gene associated an increase in the risk of having or developing a disease. In some embodiments, the method comprises (a) contacting a test compound with a model cell of any one of the described embodiments; and (b) detecting a change in a readout that is indicative of a reduction or an augmentation of a cell signaling event associated with said mutation in said disease gene, thereby developing said biologically active agent that modulates said cell signaling event associated with said disease gene.

In an aspect, the invention provides a particle delivery system or the delivery system or the virus particle of any one of any one of the above embodiments or the cell of any one of the above embodiments for use in medicine or in therapy; or for use in a method of modifying an organism or a non-human organism by manipulation of a target sequence in a genomic locus associated with a disease or disorder; or for use in a method of treating or inhibiting a condition caused by one or more mutations in a genetic locus associated with a disease in a eukaryotic organism or a non-human organism; or for use in in vitro, ex vivo or in vivo gene or genome editing; or for use in in vitro, ex vivo or in vivo gene therapy.

In an aspect, the invention provides a method of treating or inhibiting a condition or a disease caused by one or more mutations in a genomic locus in a eukaryotic organism or a non-human organism comprising manipulation of a target sequence within a coding, non-coding or regulatory element of said genomic locus in a target sequence in a subject or a non-human subject in need thereof comprising modifying the subject or a non-human subject by manipulation of the target sequence and wherein the condition or disease is susceptible to treatment or inhibition by manipulation of the target sequence comprising providing treatment comprising delivering a composition comprising the particle delivery system or the delivery system or the virus particle of any one of the above embodiment or the cell of any one of the above embodiment.

In an aspect, the invention provides methods for the use of the particle delivery system or the delivery system or the virus particle of any one of the above embodiment or the cell of any one of the above embodiment in the manufacture of a medicament for in vitro, ex vivo or in vivo gene or genome editing or for use in in vitro, ex vivo or in vivo gene therapy or for use in a method of modifying an organism or a non-human organism by manipulation of a target sequence in a genomic locus associated with a disease or in a method of treating or inhibiting a condition or disease caused by one or more mutations in a genomic locus in a eukaryotic organism or a non-human organism.

In an aspect, the invention provides a method of individualized or personalized treatment of a genetic disease in a subject in need of such treatment comprising:
(a) introducing one or more mutations ex vivo in a tissue, organ or a cell line, or in vivo in a transgenic non-human mammal, comprising delivering to cell(s) of the tissue, organ, cell or mammal a composition comprising the particle delivery system or the delivery system or the virus particle of any one of the above embodiment or the cell of any one of the above embodiment, wherein the specific mutations or precise sequence substitutions are or have been correlated to the genetic disease;
(b) testing treatment(s) for the genetic disease on the cells to which the vector has been delivered that have the specific mutations or precise sequence substitutions correlated to the genetic disease; and
(c) treating the subject based on results from the testing of treatment(s) of step (b).

In an aspect, the invention provides a method of modeling a disease associated with a genomic locus in a eukaryotic organism or a non-human organism comprising manipulation of a target sequence within a coding, non-coding or regulatory element of said genomic locus comprising delivering a non-naturally occurring or engineered composition comprising a viral vector system comprising one or more viral vectors operably encoding a composition for expression thereof, wherein the composition comprises particle delivery system or the delivery system or the virus particle of any one of the above embodiments or the cell of any one of the above embodiment.

In an aspect, the method provides a method of modifying an organism or a non-human organism by manipulation of a target sequence in a genomic locus of interest comprising administering a composition comprising the particle delivery system or the delivery system or the virus particle of any one of the above embodiments or the cell of any one of the above embodiments.

In some embodiments, methods comprise delivery of one or more components of the system, for example the oligonucleotides and/or polypeptitde components of the system, for example an ssODN by a vector, e.g., plasmid or viral vector is delivered to the tissue of interest by, for example, an intramuscular injection, while other times the delivery is via intravenous, transdermal, intranasal, oral, mucosal, or other delivery methods. In an aspect, the components can be sel assembling allowing the delivery of the components of the system to be delivered together or by separate means. Such delivery may be either via a single dose, or multiple doses. One skilled in the art understands that the actual dosage to be delivered herein may vary greatly depending upon a variety of factors, such as the vector choice, the target cell, organism, or tissue, the general condition of the subject to be treated, the degree of transformation/modification sought, the administration route, the administration mode, the type of transformation/modification sought, etc.

Such a dosage may further contain, for example, a carrier (water, saline, ethanol, glycerol, lactose, sucrose, calcium phosphate, gelatin, dextran, agar, pectin, peanut oil, sesame oil, etc.), a diluent, a pharmaceutically-acceptable carrier (e.g., phosphate-buffered saline), a pharmaceutically-acceptable excipient, and/or other compounds known in the art. The dosage may further contain one or more pharmaceutically acceptable salts such as, for example, a mineral acid salt such as a hydrochloride, a hydrobromide, a phosphate, a sulfate, etc.; and the salts of organic acids such as acetates, propionates, malonates, benzoates, etc. Additionally, auxiliary substances, such as wetting or emulsifying agents, pH buffering substances, gels or gelling materials, flavorings, colorants, microspheres, polymers, suspension agents, etc. may also be present herein. In addition, one or more other conventional pharmaceutical ingredients, such as preservatives, humectants, suspending agents, surfactants, antioxidants, anticaking agents, fillers, chelating agents, coating agents, chemical stabilizers, etc. may also be present, especially if the dosage form is a reconstitutable form. Suitable exemplary ingredients include microcrystalline cellulose, carboxymethylcellulose sodium, polysorbate 80, phenylethyl alcohol, chlorobutanol, potassium sorbate, sorbic acid, sulfur dioxide, propyl gallate, the parabens, ethyl vanillin, glycerin, phenol, parachlorophenol, gelatin, albumin and a combination thereof. A thorough discussion of pharmaceutically acceptable excipients is available in REMINGTON-S PHARMACEUTICAL SCIENCES (Mack Pub. Co., N.J. 1991) which is incorporated by reference herein.

In an embodiment herein, the delivery is via a plasmid. In such plasmid compositions, the dosage should be a sufficient amount of plasmid to elicit a response. For instance, suitable quantities of plasmid DNA in plasmid compositions can be from about 0.1 to about 2 mg, or from about 1 μg to about 10 μg per 70 kg individual. Plasmids of the invention will generally comprise (i) a promoter; (ii) a sequence encoding a CRISPR enzyme, operably linked to said promoter; (iii) a selectable marker; (iv) an origin of replication; and (v) a transcription terminator downstream of and operably linked to (ii). The plasmid can also encode the RNA components of a CRISPR complex, but one or more of these may instead be encoded on a different vector.

The doses herein are based on an average 70 kg individual. The frequency of administration is within the ambit of the medical or veterinary practitioner (e.g., physician, veterinarian), or scientist skilled in the art. It is also noted that mice used in experiments are typically about 20 g and from mice experiments one can scale up to a 70 kg individual.

In some embodiments, methods of delivery of systems in vivo can be accomplished by delivery of PNAs using Poly(lactic co-glycolic acids) (PLGA) nanoparticles as detailed elsewhere herein.

In some embodiments the RNA molecules of the invention are delivered in liposome or lipofectin formulations and the like and can be prepared by methods well known to those skilled in the art. Such methods are described, for example, in U.S. Pat. Nos. 5,593,972, 5,589,466, and 5,580,859, which are herein incorporated by reference. Delivery systems aimed specifically at the enhanced and improved delivery of siRNA into mammalian cells have been developed, (see, for example, Shen et al FEBS Let. 2003, 539:111-114; Xia et al., Nat. Biotech. 2002, 20:1006-1010; Reich et al., Mol. Vision. 2003, 9: 210-216; Sorensen et al., J. Mol. Biol. 2003, 327: 761-766; Lewis et al., Nat. Gen. 2002, 32: 107-108 and Simeoni et al., NAR 2003, 31, 11: 2717-2724) and may be applied to the present invention. siRNA has recently been successfully used for inhibition of gene expression in primates (see for example. Tolentino et al., Retina 24(4):660 which may also be applied to the present invention.

Means of delivery of RNA also preferred include delivery of RNA via particles (Cho, S., Goldberg, M., Son, S., Xu, Q., Yang, F., Mei, Y., Bogatyrev, S., Langer, R. and Anderson, D., Lipid-like nanoparticles for small interfering RNA delivery to endothelial cells, Advanced Functional Materials, 19: 3112-3118, 2010) or exosomes (Schroeder, A., Levins, C., Cortez, C., Langer, R., and Anderson, D., Lipid-based nanotherapeutics for siRNA delivery, Journal of Internal Medicine, 267: 9-21, 2010, PMID: 20059641). Indeed, exosomes have been shown to be particularly useful in delivery siRNA, a system with some parallels to the CRISPR system. For instance, El-Andaloussi S, et al. (“Exosome-mediated delivery of siRNA in vitro and in vivo.” Nat Protoc. 2012 December; 7(12):2112-26. doi: 10.1038/nprot.2012.131. Epub 2012 Nov. 15) describe how exosomes are promising tools for drug delivery across different biological barriers and can be harnessed for delivery of siRNA in vitro and in vivo. Their approach is to generate targeted exosomes through transfection of an expression vector, comprising an exosomal protein fused with a peptide ligand. The exosomes are then purify and characterized from transfected cell supernatant, then RNA is loaded into the exosomes. Delivery or administration according to the invention can be performed with exosomes, in particular but not limited to the brain. Vitamin E (α-tocopherol) may be conjugated with CRISPR Cas and delivered to the brain along with high density lipoprotein (HDL), for example in a similar manner as was done by Uno et al. (HUMAN GENE THERAPY 22:711-719 (June 2011)) for delivering short-interfering RNA (siRNA) to the brain. Mice were infused via Osmotic minipumps (model 1007D; Alzet, Cupertino, Calif.) filled with phosphate-buffered saline (PBS) or free TocsiBACE or Toc-siBACE/HDL and connected with Brain Infusion Kit 3 (Alzet). A brain-infusion cannula was placed about 0.5 mm posterior to the bregma at midline for infusion into the dorsal third ventricle. Uno et al. found that as little as 3 nmol of Toc-siRNA with HDL could induce a target reduction in comparable degree by the same ICV infusion method. A similar dosage of CRISPR Cas conjugated to α-tocopherol and co-administered with HDL targeted to the brain may be contemplated for humans in the present invention, for example, about 3 nmol to about 3 μmol of CRISPR Cas targeted to the brain may be contemplated. Zou et al. ((HUMAN GENE THERAPY 22:465-475 (April 2011)) describes a method of lentiviral-mediated delivery of short-hairpin RNAs targeting PKCγ for in vivo gene silencing in the spinal cord of rats. Zou et al. administered about 10 μl of a recombinant lentivirus having a titer of 1×109 transducing units (TU)/ml by an intrathecal catheter. A similar dosage of CRISPR Cas expressed in a lentiviral vector targeted to the brain may be contemplated for humans in the present invention, for example, about 10-50 ml of CRISPR Cas targeted to the brain in a lentivirus having a titer of 1×109 transducing units (TU)/ml may be contemplated. Exemplary compounds and particles for use in delivery are described in International Patent Publication WO 2019/135816 at [0643]-[0673], specifically incorporated herein by reference.

For example, mRNA and guide RNA may be delivered simultaneously using nanoparticles or lipid envelopes. Similarly, synthetic nucleic acid modifying systems comprising DNA readers and one or more effector components can be delivered by nanoparticles

### Target Sequences

The current invention comprehends the use of the compositions of the current invention to establish and utilize conditional or inducible nucleic acid modifying transgenic cell/animals; see, e.g., Platt et al., Cell (2014), 159(2): 440-455, or PCT patent publications cited herein, such as WO 2014/093622 (PCT/US2013/074667). For example, cells or animals such as non-human animals, e.g., vertebrates or mammals, such as rodents, e.g., mice, rats, or other laboratory or field animals, e.g., cats, dogs, sheep, etc., may be ‘knock-in’ whereby the animal conditionally or inducibly expresses nucleic acid modifying protein akin to Platt et al. The target cell or animal thus comprises the nucleic acid modifying protein comprising one or more domains of a Cas protein conditionally or inducibly (e.g., in the form of Cre dependent constructs), on expression of a vector introduced into the target cell, the vector expresses that which induces or gives rise to the condition of the nucleic acid modifying protein expression in the target cell. By applying the teaching and compositions as defined herein with the known method of creating a nucleic acid modifying complex, inducible genomic events are also an aspect of the current invention. Examples of such inducible events have been described herein elsewhere.

In some embodiments, phenotypic alteration is preferably the result of genome modification when a genetic disease is targeted, especially in methods of therapy and preferably where a repair template is provided to correct or alter the phenotype.

In some embodiments diseases that may be targeted include those concerned with disease-causing splice defects.

In some embodiments, cellular targets include Hemopoietic Stem/Progenitor Cells (CD34+); Human T cells; and Eye (retinal cells)—for example photoreceptor precursor cells.

In some embodiments Gene targets include: Human Beta Globin—HBB (for treating Sickle Cell Anemia, including by stimulating gene-conversion (using closely related HBD gene as an endogenous template)); CD3 (T-Cells); and CEP920—retina (eye).

In some embodiments disease targets also include: cancer; Sickle Cell Anemia (based on a point mutation); HBV, HIV; Beta-Thalassemia; and ophthalmic or ocular disease—for example Leber Congenital Amaurosis (LCA)-causing Splice Defect.

Nucleic acid modifying systems of the present invention can be used for selective perturbations, precise genome targeting technologies, including reverse engineering of causal genetic variations, rectifying genomic alterations, and for use in disease models. Nucleic acid modifying systems or complexes can target nucleic acid molecules, e.g., nucleic acid modifying complexes can target and cleave or nick or simply sit upon a target DNA molecule. Such systems or complexes are amenable for achieving tissue-specific and temporally controlled targeted deletion of candidate disease genes. Examples include but are not limited to genes involved in cholesterol and fatty acid metabolism, amyloid diseases, dominant negative diseases, latent viral infections, among other disorders. Accordingly, target sequences for such systems or complexes can be in candidate disease genes, e.g.:

TABLE 2

| Disease | GENE | SPACER | PAM | Mechanism | References |
|---|---|---|---|---|---|
| Hypercholest- | HMG-CR | GCCAAATTGGACGACC | CGG | Knockout | Fluvastatin: a review of its |
| erolemia | | CTCG | | | pharmacology and use in the |
| | | (SEQ ID NO: 9) | | | management of hypercholest- |
| | | | | | erolaemia. (Plosker GL et al. |
| | | | | | Drugs 1996, 51(3): 433-459) |
| Hypercholest- | SQLE | CGAGGAGACCCCCGTT | TGG | Knockout | Potential role of nonstatin |
| erolemia | | TCGG | | | cholesterol lowering agents |
| | | (SEQ ID NO: 10) | | | (Trapani et al. IUBMB Life, |
| | | | | | Volume 63, Issue 11, pages |
| | | | | | 964-971, Nov. 2011) |
| Hyperlipidemia | DGAT1 | CCCGCCGCCGCCGTGG | AGG | Knockout | DGAT1 inhibitors as anti- |
| | | CTCG | | | obesity and anti-diabetic |
| | | (SEQ ID NO: 11) | | | agents. (Birch AM et al. |
| | | | | | Current Opinion in Drug |
| | | | | | Discovery & Development |
| | | | | | [2010, 13(4): 489-496] |
| Leukemia | BCR- | TGAGCTCTACGAGATC | AGG | Knockout | Killing of leukemic cell with |
| | ABL | CACA | | | a BCR/ABL fusion gene by RNA |
| | | (SEQ ID NO: 12) | | | interference (RNAi). (Fuchs |
| | | | | | et al. Oncogene 2002, |
| | | | | | 21(37): 5716-5724) |

Thus, the present invention, with regard to nucleic acid modifying protein or nucleic acid modifying complexes contemplates correction of hematopoietic disorders. For example, Severe Combined Immune Deficiency (SCID) results from a defect in lymphocytes T maturation, always associated with a functional defect in lymphocytes B (Cavazzana-Calvo et al., Annu. Rev. Med., 2005, 56, 585-602; Fischer et al., Immunol. Rev., 2005, 203, 98-109). In the case of Adenosine Deaminase (ADA) deficiency, one of the SCID forms, patients can be treated by injection of recombinant Adenosine Deaminase enzyme. Since the ADA gene has been shown to be mutated in SCID patients (Giblett et al., Lancet, 1972, 2, 1067-1069), several other genes involved in SCID have been identified (Cavazzana-Calvo et al., Annu. Rev. Med., 2005, 56, 585-602; Fischer et al., Immunol. Rev., 2005, 203, 98-109). There are four major causes for SCID: (i) the most frequent form of SCID, SCID-X1 (X-linked SCID or X-SCID), is caused by mutation in the IL2RG gene, resulting in the absence of mature T lymphocytes and NK cells. IL2RG encodes the gamma C protein (Noguchi, et al., Cell, 1993, 73, 147-157), a common component of at least five interleukin receptor complexes. These receptors activate several targets through the JAK3 kinase (Macchi et al., Nature, 1995, 377, 65-68), which inactivation results in the same syndrome as gamma C inactivation; (ii) mutation in the ADA gene results in a defect in purine metabolism that is lethal for lymphocyte precursors, which in turn results in the quasi absence of B, T and NK cells; (iii) V(D)J recombination is an essential step in the maturation of immunoglobulins and T lymphocytes receptors (TCRs). Mutations in Recombination Activating Gene 1 and 2 (RAG1 and RAG2) and Artemis, three genes involved in this process, result in the absence of mature T and B lymphocytes; and (iv) Mutations in other genes such as CD45, involved in T cell specific signaling have also been reported, although they represent a minority of cases (Cavazzana-Calvo et al., Annu. Rev. Med., 2005, 56, 585-602; Fischer et al., Immunol. Rev., 2005, 203, 98-109). In aspect of the invention, relating to CRISPR or CRISPR-Cas complexes contemplates system, the invention contemplates that it may be used to correct ocular defects that arise from several genetic mutations further described in Genetic Diseases of the Eye, Second Edition, edited by Elias I. Traboulsi, Oxford University Press, 2012. Non-limiting examples of ocular defects to be corrected include macular degeneration (MD), retinitis pigmentosa (RP). Non-limiting examples of genes and proteins associated with ocular defects include but are not limited to the following proteins: (ABCA4) ATP-binding cassette, sub-family A (ABC1), member 4 ACHM1 achromatopsia (rod monochromacy) 1 ApoE Apolipoprotein E (ApoE) C1QTNF5 (CTRPS) Clq and tumor necrosis factor related protein 5 (C1QTNF5) C2 Complement component 2 (C2) C3 Complement components (C3) CCL2 Chemokine (C—C motif) Ligand 2 (CCL2) CCR2 Chemokine (C—C motif) receptor 2 (CCR2) CD36 Cluster of Differentiation 36 CFB Complement factor B CFH Complement factor CFH H CFHR1 complement factor H-related 1 CFHR3 complement factor H-related 3 CNGB3 cyclic nucleotide gated channel beta 3 CP ceruloplasmin (CP) CRP C reactive protein (CRP) CST3 cystatin C or cystatin 3 (CST3) CTSD Cathepsin D (CTSD) CX3CR1 chemokine (C-X3-C motif) receptor 1 ELOVL4 Elongation of very long chain fatty acids 4 ERCC6 excision repair cross-complementing rodent repair deficiency, complementation group 6 FBLNS Fibulin-5 FBLNS Fibulin 5 FBLN6 Fibulin 6 FSCN2 fascin (FSCN2) HMCN1 Hemicentrin 1 HMCN1 hemicentin 1 HTRA1 HtrA serine peptidase 1 (HTRA1) HTRA1 HtrA serine peptidase 1 IL-6 Interleukin 6 IL-8 Interleukin 8 LOC387715 Hypothetical protein PLEKHA1 Pleckstrin homology domain-containing family A member 1 (PLEKHA1) PROM1 Prominin 1 (PROM1 or CD133) PRPH2 Peripherin-2 RPGR retinitis pigmentosa GTPase regulator SERPING1 serpin peptidase inhibitor, clade G, member 1 (C1-inhibitor) TCOF1 Treacle TIMP3 Metalloproteinase inhibitor 3 (TIMP3) TLR3 Toll-like receptor 3 The present invention, with regard to CRISPR or CRISPR-Cas complexes contemplates also contemplates delivering to the heart. For the heart, a myocardium tropic adena-associated virus (AAVM) is preferred, in particular AAVM41 which showed preferential gene transfer in the heart (see, e.g., Lin-Yanga et al., PNAS, Mar. 10, 2009, vol. 106, no. 10). For example, US Patent Publication No. 20110023139, describes use of zinc finger nucleases to genetically modify cells, animals and proteins associated with cardiovascular disease. Cardiovascular diseases generally include high blood pressure, heart attacks, heart failure, and stroke and TIA. By way of example, the chromosomal sequence may comprise, but is not limited to, IL1B (interleukin 1, beta), XDH (xanthine dehydrogenase), TP53 (tumor protein p53), PTGIS (prostaglandin 12 (prostacyclin) synthase), MB (myoglobin), IL4 (interleukin 4), ANGPT1 (angiopoietin 1), ABCG8 (ATP-binding cassette, sub-family G (WHITE), member 8), CTSK (cathepsin K), PTGIR (prostaglandin 12 (prostacyclin) receptor (IP)), KCNJ11 (potassium inwardly-rectifying channel, subfamily J, member 11), INS (insulin), CRP (C-reactive protein, pentraxin-related), PDGFRB (platelet-derived growth factor receptor, beta polypeptide), CCNA2 (cyclin A2), PDGFB (platelet-derived growth factor beta polypeptide (simian sarcoma viral (v-sis) oncogene homolog)), KCNJS (potassium inwardly-rectifying channel, subfamily J, member 5), KCNN3 (potassium intermediate/small conductance calcium-activated channel, subfamily N, member 3), CAPN10 (calpain 10), PTGES (prostaglandin E synthase), ADRA2B (adrenergic, alpha-2B-, receptor), ABCGS (ATP-binding cassette, sub-family G (WHITE), member 5), PRDX2 (peroxiredoxin 2), CAPNS (calpain 5), PARP14 (poly (ADP-ribose) polymerase family, member 14), MEX3C (mex-3 homolog C (C. elegans)), ACE angiotensin I converting enzyme (peptidyl-dipeptidase A) 1), TNF (tumor necrosis factor (TNF superfamily, member 2)), IL6 (interleukin 6 (interferon, beta 2)), STN (statin), SERPINE1 (serpin peptidase inhibitor, clade E (nexin, plasminogen activator inhibitor type 1), member 1), ALB (albumin), ADIPOQ (adiponectin, C1Q and collagen domain containing), APOB (apolipoprotein B (including Ag(x) antigen)), APOE (apolipoprotein E), LEP (leptin), MTHFR (5,10-methylenetetrahydrofolate reductase (NADPH)), APOA1 (apolipoprotein A-I), EDN1 (endothelin 1), NPPB (natriuretic peptide precursor B), NOS3 (nitric oxide synthase 3 (endothelial cell)), PPARG (peroxisome proliferator-activated receptor gamma), PLAT (plasminogen activator, tissue), PTGS2 (prostaglandin-endoperoxide synthase 2 (prostaglandin G/H synthase and cyclooxygenase)), CETP (cholesteryl ester transfer protein, plasma), AGTR1 (angiotensin II receptor, type 1), HMGCR (3-hydroxy-3-methylglutaryl-Coenzyme A reductase), IGF1 (insulin-like growth factor 1 (somatomedin C)), SELE (selectin E), REN (renin), PPARA (peroxisome proliferator-activated receptor alpha), PON1 (paraoxonase 1), KNG1 (kininogen 1), CCL2 (chemokine (C—C motif) ligand 2), LPL (lipoprotein lipase), VWF (von Willebrand factor), F2 (coagulation factor II (thrombin)), ICAM1 (intercellular adhesion molecule 1), TGFB1 (transforming growth factor, beta 1), NPPA (natriuretic peptide precursor A), IL10 (interleukin 10), EPO (erythropoietin), SOD1 (superoxide dismutase 1, soluble), VCAM1 (vascular cell adhesion molecule 1), IFNG (interferon, gamma), LPA (lipoprotein, Lp(a)), MPO (myeloperoxidase), ESR1 (estrogen receptor 1), MAPK1 (mitogen-activated protein kinase 1), HP (haptoglobin), F3 (coagulation factor III (thromboplastin, tissue factor)), CST3 (cystatin C), COG2 (component of oligomeric golgi complex 2), MMP9 (matrix metallopeptidase 9 (gelatinase B, 92 kDa gelatinase, 92 kDa type IV collagenase)), SERPINC1 (serpin peptidase inhibitor, clade C (antithrombin), member 1), F8 (coagulation factor VIII, procoagulant component), HMOX1 (heme oxygenase (decycling) 1), APOC3 (apolipoprotein C-III), IL8 (interleukin 8), PROK1 (prokineticin 1), CBS (cystathionine-beta-synthase), NOS2 (nitric oxide synthase 2, inducible), TLR4 (toll-like receptor 4), SELP (selectin P (granule membrane protein 140 kDa, antigen CD62)), ABCA1 (ATP-binding cassette, sub-family A (ABC1), member 1), AGT (angiotensinogen (serpin peptidase inhibitor, clade A, member 8)), LDLR (low density lipoprotein receptor), GPT (glutamic-pyruvate transaminase (alanine aminotransferase)), VEGFA (vascular endothelial growth factor A), NR3C2 (nuclear receptor subfamily 3, group C, member 2), IL18 (interleukin 18 (interferon-gamma-inducing factor)), NOS1 (nitric oxide synthase 1 (neuronal)), NR3C1 (nuclear receptor subfamily 3, group C, member 1 (glucocorticoid receptor)), FGB (fibrinogen beta chain), HGF (hepatocyte growth factor (hepapoietin A; scatter factor)), ILIA (interleukin 1, alpha), RETN (resistin), AKT1 (v-akt murine thymoma viral oncogene homolog 1), LIPC (lipase, hepatic), HSPD1 (heat shock 60 kDa protein 1 (chaperonin)), MAPK14 (mitogen-activated protein kinase 14), SPP1 (secreted phosphoprotein 1), ITGB3 (integrin, beta 3 (platelet glycoprotein 111a, antigen CD61)), CAT (catalase), UTS2 (urotensin 2), THBD (thrombomodulin), F10 (coagulation factor X), CP (ceruloplasmin (ferroxidase)), TNFRSF11B (tumor necrosis factor receptor superfamily, member 11b), EDNRA (endothelin receptor type A), EGFR (epidermal growth factor receptor (erythroblastic leukemia viral (v-erb-b) oncogene homolog, avian)), MMP2 (matrix metallopeptidase 2 (gelatinase A, 72 kDa gelatinase, 72 kDa type IV collagenase)), PLG (plasminogen), NPY (neuropeptide Y), RHOD (ras homolog gene family, member D), MAPK8 (mitogen-activated protein kinase 8), MYC (v-myc myelocytomatosis viral oncogene homolog (avian)), FN1 (fibronectin 1), CMA1 (chymase 1, mast cell), PLAU (plasminogen activator, urokinase), GNB3 (guanine nucleotide binding protein (G protein), beta polypeptide 3), ADRB2 (adrenergic, beta-2-, receptor, surface), APOA5 (apolipoprotein A-V), SOD2 (superoxide dismutase 2, mitochondrial), F5 (coagulation factor V (proaccelerin, labile factor)), VDR (vitamin D (1,25-dihydroxyvitamin D3) receptor), ALOX5 (arachidonate 5-lipoxygenase), HLA-DRB1 (major histocompatibility complex, class II, DR beta 1), PARP1 (poly (ADP-ribose) polymerase 1), CD40LG (CD40 ligand), PON2 (paraoxonase 2), AGER (advanced glycosylation end product-specific receptor), IRS1 (insulin receptor substrate 1), PTGS1 (prostaglandin-endoperoxide synthase 1 (prostaglandin G/H synthase and cyclooxygenase)), ECE1 (endothelin converting enzyme 1), F7 (coagulation factor VII (serum prothrombin conversion accelerator)), URN (interleukin 1 receptor antagonist), EPHX2 (epoxide hydrolase 2, cytoplasmic), IGFBP1 (insulin-like growth factor binding protein 1), MAPK10 (mitogen-activated protein kinase 10), FAS (Fas (TNF receptor superfamily, member 6)), ABCB1 (ATP-binding cassette, sub-family B (MDR/TAP), member 1), JUN (jun oncogene), IGFBP3 (insulin-like growth factor binding protein 3), CD14 (CD14 molecule), PDESA (phosphodiesterase 5A, cGMP-specific), AGTR2 (angiotensin II receptor, type 2), CD40 (CD40 molecule, TNF receptor superfamily member 5), LCAT (lecithin-cholesterol acyltransferase), CCRS (chemokine (C—C motif) receptor 5), MMP1 (matrix metallopeptidase 1 (interstitial collagenase)), TIMP1 (TIMP metallopeptidase inhibitor 1), ADM (adrenomedullin), DYT10 (dystonia 10), STAT3 (signal transducer and activator of transcription 3 (acute-phase response factor)), MMP3 (matrix metallopeptidase 3 (stromelysin 1, progelatinase)), ELN (elastin), USF1 (upstream transcription factor 1), CFH (complement factor H), HSPA4 (heat shock 70 kDa protein 4), MMP12 (matrix metallopeptidase 12 (macrophage elastase)), MME (membrane metallo-endopeptidase), F2R (coagulation factor II (thrombin) receptor), SELL (selectin L), CTSB (cathepsin B), ANXAS (annexin A5), ADRB1 (adrenergic, beta-1-, receptor), CYBA (cytochrome b-245, alpha polypeptide), FGA (fibrinogen alpha chain), GGT1 (gamma-glutamyltransferase 1), LIPG (lipase, endothelial), HIF1A (hypoxia inducible factor 1, alpha subunit (basic helix-loop-helix transcription factor)), CXCR4 (chemokine (C—X—C motif) receptor 4), PROC (protein C (inactivator of coagulation factors Va and VIIIa)), SCARB1 (scavenger receptor class B, member 1), CD79A (CD79a molecule, immunoglobulin-associated alpha), PLTP (phospholipid transfer protein), ADD1 (adducin 1 (alpha)), FGG (fibrinogen gamma chain), SAA1 (serum amyloid A1), KCNH2 (potassium voltage-gated channel, subfamily H (eag-related), member 2), DPP4 (dipeptidyl-peptidase 4), G6PD (glucose-6-phosphate dehydrogenase), NPR1 (natriuretic peptide receptor A/guanylate cyclase A (atrionatriuretic peptide receptor A)), VTN (vitronectin), KIAA0101 (KIAA0101), FOS (FBJ murine osteosarcoma viral oncogene homolog), TLR2 (toll-like receptor 2), PPIG (peptidylprolyl isomerase G (cyclophilin G)), IL1R1 (interleukin 1 receptor, type I), AR (androgen receptor), CYP1A1 (cytochrome P450, family 1, subfamily A, polypeptide 1), SERPINA1 (serpin peptidase inhibitor, clade A (alpha-1 antiproteinase, antitrypsin), member 1), MTR (5-methyltetrahydrofolate-homocysteine methyltransferase), RBP4 (retinol binding protein 4, plasma), APOA4 (apolipoprotein A-IV), CDKN2A (cyclin-dependent kinase inhibitor 2A (melanoma, p16, inhibits CDK4)), FGF2 (fibroblast growth factor 2 (basic)), EDNRB (endothelin receptor type B), ITGA2 (integrin, alpha 2 (CD49B, alpha 2 subunit of VLA-2 receptor)), CABIN′ (calcineurin binding protein 1), SHBG (sex hormone-binding globulin), HMGB1 (high-mobility group box 1), HSP90B2P (heat shock protein 90 kDa beta (Grp94), member 2 (pseudogene)), CYP3A4 (cytochrome P450, family 3, subfamily A, polypeptide 4), GJA1 (gap junction protein, alpha 1, 43 kDa), CAV1 (caveolin 1, caveolae protein, 22 kDa), ESR2 (estrogen receptor 2 (ER beta)), LTA (lymphotoxin alpha (TNF superfamily, member 1)), GDF15 (growth differentiation factor 15), BDNF (brain-derived neurotrophic factor), CYP2D6 (cytochrome P450, family 2, subfamily D, polypeptide 6), NGF (nerve growth factor (beta polypeptide)), SP1 (Sp1 transcription factor), TGIF1 (TGFB-induced factor homeobox 1), SRC (v-src sarcoma (Schmidt-Ruppin A-2) viral oncogene homolog (avian)), EGF (epidermal growth factor (beta-urogastrone)), PIK3CG (phosphoinositide-3-kinase, catalytic, gamma polypeptide), HLA-A (major histocompatibility complex, class I, A), KCNQ1 (potassium voltage-gated channel, KQT-like subfamily, member 1), CNR1 (cannabinoid receptor 1 (brain)), FBN1 (fibrillin 1), CHKA (choline kinase alpha), BEST1 (bestrophin 1), APP (amyloid beta (A4) precursor protein), CTNNB1 (catenin (cadherin-associated protein), beta 1, 88 kDa), IL2 (interleukin 2), CD36 (CD36 molecule (thrombospondin receptor)), PRKAB1 (protein kinase, AMP-activated, beta 1 non-catalytic subunit), TPO (thyroid peroxidase), ALDH7A1 (aldehyde dehydrogenase 7 family, member A1), CX3CR1 (chemokine (C-X3-C motif) receptor 1), TH (tyrosine hydroxylase), F9 (coagulation factor IX), GH1 (growth hormone 1), TF (transferrin), HFE (hemochromatosis), IL17A (interleukin 17A), PTEN (phosphatase and tensin homolog), GSTM1 (glutathione S-transferase mu 1), DMD (dystrophin), GATA4 (GATA binding protein 4), F13A1 (coagulation factor XIII, A1 polypeptide), TTR (transthyretin), FABP4 (fatty acid binding protein 4, adipocyte), PON3 (paraoxonase 3), APOC1 (apolipoprotein C-I), INSR (insulin receptor), TNFRSF1B (tumor necrosis factor receptor superfamily, member 1B), HTR2A (5-hydroxytryptamine (serotonin) receptor 2A), CSF3 (colony stimulating factor 3 (granulocyte)), CYP2C9 (cytochrome P450, family 2, subfamily C, polypeptide 9), TXN (thioredoxin), CYP11B2 (cytochrome P450, family 11, subfamily B, polypeptide 2), PTH (parathyroid hormone), CSF2 (colony stimulating factor 2 (granulocyte-macrophage)), KDR (kinase insert domain receptor (a type III receptor tyrosine kinase)), PLA2G2A (phospholipase A2, group IIA (platelets, synovial fluid)), B2M (beta-2-microglobulin), THBS1 (thrombospondin 1), GCG (glucagon), RHOA (ras homolog gene family, member A), ALDH2 (aldehyde dehydrogenase 2 family (mitochondrial)), TCF7L2 (transcription factor 7-like 2 (T-cell specific, HMG-box)), BDKRB2 (bradykinin receptor B2), NFE2L2 (nuclear factor (erythroid-derived 2)-like 2), NOTCH1 (Notch homolog 1, translocation-associated (Drosophila)), UGT1A1 (UDP glucuronosyltransferase 1 family, polypeptide A1), IFNA1 (interferon, alpha 1), PPARD (peroxisome proliferator-activated receptor delta), SIRT1 (sirtuin (silent mating type information regulation 2 homolog) 1 (S. cerevisiae)), GNRH1 (gonadotropin-releasing hormone 1 (luteinizing-releasing hormone)), PAPPA (pregnancy-associated plasma protein A, pappalysin 1), ARR3 (arrestin 3, retinal (X-arrestin)), NPPC (natriuretic peptide precursor C), AHSP (alpha hemoglobin stabilizing protein), PTK2 (PTK2 protein tyrosine kinase 2), IL13 (interleukin 13), MTOR (mechanistic target of rapamycin (serine/threonine kinase)), ITGB2 (integrin, beta 2 (complement component 3 receptor 3 and 4 subunit)), GSTT1 (glutathione 5-transferase theta 1), IL6ST (interleukin 6 signal transducer (gp130, oncostatin M receptor)), CPB2 (carboxypeptidase B2 (plasma)), CYP1A2 (cytochrome P450, family 1, subfamily A, polypeptide 2), HNF4A (hepatocyte nuclear factor 4, alpha), SLC6A4 (solute carrier family 6 (neurotransmitter transporter, serotonin), member 4), PLA2G6 (phospholipase A2, group VI (cytosolic, calcium-independent)), TNFSF11 (tumor necrosis factor (ligand) superfamily, member 11), SLC8A1 (solute carrier family 8 (sodium/calcium exchanger), member 1), F2RL1 (coagulation factor II (thrombin) receptor-like 1), AKR1A1 (aldo-keto reductase family 1, member A1 (aldehyde reductase)), ALDH9A1 (aldehyde dehydrogenase 9 family, member A1), BGLAP (bone gamma-carboxyglutamate (gla) protein), MTTP (microsomal triglyceride transfer protein), MTRR (5-methyltetrahydrofolate-homocysteine methyltransferase reductase), SULT1A3 (sulfotransferase family, cytosolic, 1A, phenol-preferring, member 3), RAGE (renal tumor antigen), C4B (complement component 4B (Chido blood group), P2RY12 (purinergic receptor P2Y, G-protein coupled, 12), RNLS (renalase, FAD-dependent amine oxidase), CREB1 (cAMP responsive element binding protein 1), POMC (proopiomelanocortin), RAC1 (ras-related C3 botulinum toxin substrate 1 (rho family, small GTP binding protein Rad)), LMNA (lamin NC), CD59 (CD59 molecule, complement regulatory protein), SCNSA (sodium channel, voltage-gated, type V, alpha subunit), CYP1B1 (cytochrome P450, family 1, subfamily B, polypeptide 1), MIF (macrophage migration inhibitory factor (glycosylation-inhibiting factor)), MMP13 (matrix metallopeptidase 13 (collagenase 3)), TIMP2 (TIMP metallopeptidase inhibitor 2), CYP19A1 (cytochrome P450, family 19, subfamily A, polypeptide 1), CYP21A2 (cytochrome P450, family 21, subfamily A, polypeptide 2), PTPN22 (protein tyrosine phosphatase, non-receptor type 22 (lymphoid)), MYH14 (myosin, heavy chain 14, non-muscle), MBL2 (mannose-binding lectin (protein C) 2, soluble (opsonic defect)), SELPLG (selectin P ligand), AOC3 (amine oxidase, copper containing 3 (vascular adhesion protein 1)), CTSL1 (cathepsin L1), PCNA (proliferating cell nuclear antigen), IGF2 (insulin-like growth factor 2 (somatomedin A)), ITGB1 (integrin, beta 1 (fibronectin receptor, beta polypeptide, antigen CD29 includes MDF2, MSK12)), CAST (calpastatin), CXCL12 (chemokine (C—X—C motif) ligand 12 (stromal cell-derived factor 1)), IGHE (immunoglobulin heavy constant epsilon), KCNE1 (potassium voltage-gated channel, Isk-related family, member 1), TFRC (transferrin receptor (p90, CD71)), COL1A1 (collagen, type I, alpha 1), COL1A2 (collagen, type I, alpha 2), IL2RB (interleukin 2 receptor, beta), PLA2G10 (phospholipase A2, group X), ANGPT2 (angiopoietin 2), PROCR (protein C receptor, endothelial (EPCR)), NOX4 (NADPH oxidase 4), HAMP (hepcidin antimicrobial peptide), PTPN11 (protein tyrosine phosphatase, non-receptor type 11), SLC2A1 (solute carrier family 2 (facilitated glucose transporter), member 1), IL2RA (interleukin 2 receptor, alpha), CCL5 (chemokine (C—C motif) ligand 5), IRF1 (interferon regulatory factor 1), CFLAR (CASP8 and FADD-like apoptosis regulator), CALCA (calcitonin-related polypeptide alpha), EIF4E (eukaryotic translation initiation factor 4E), GSTP1 (glutathione S-transferase pi 1), JAK2 (Janus kinase 2), CYP3A5 (cytochrome P450, family 3, subfamily A, polypeptide 5), HSPG2 (heparan sulfate proteoglycan 2), CCL3 (chemokine (C—C motif) ligand 3), MYD88 (myeloid differentiation primary response gene (88)), VIP (vasoactive intestinal peptide), SOAT1 (sterol O-acyltransferase 1), ADRBK1 (adrenergic, beta, receptor kinase 1), NR4A2 (nuclear receptor subfamily 4, group A, member 2), MMP8 (matrix metallopeptidase 8 (neutrophil collagenase)), NPR2 (natriuretic peptide receptor B/guanylate cyclase B (atrionatriuretic peptide receptor B)), GCH1 (GTP cyclohydrolase 1), EPRS (glutamyl-prolyl-tRNA synthetase), PPARGC1A (peroxisome proliferator-activated receptor gamma, coactivator 1 alpha), F12 (coagulation factor XII (Hageman factor)), PECAM1 (platelet/endothelial cell adhesion molecule), CCL4 (chemokine (C—C motif) ligand 4), SERPINA3 (serpin peptidase inhibitor, clade A (alpha-1 antiproteinase, antitrypsin), member 3), CASR (calcium-sensing receptor), GJA5 (gap junction protein, alpha 5, 40 kDa), FABP2 (fatty acid binding protein 2, intestinal), TTF2 (transcription termination factor, RNA polymerase II), PROS1 (protein S (alpha)), CTF1 (cardiotrophin 1), SGCB (sarcoglycan, beta (43 kDa dystrophin-associated glycoprotein)), YME1L1 (YME1-like 1 (S. cerevisiae)), CAMP (cathelicidin antimicrobial peptide), ZC3H12A (zinc finger CCCH-type containing 12A), AKR1B1 (aldo-keto reductase family 1, member B1 (aldose reductase)), DES (desmin), MMP7 (matrix metallopeptidase 7 (matrilysin, uterine)), AHR (aryl hydrocarbon receptor), CSF1 (colony stimulating factor 1 (macrophage)), HDAC9 (histone deacetylase 9), CTGF (connective tissue growth factor), KCNMA1 (potassium large conductance calcium-activated channel, subfamily M, alpha member 1), UGT1A (UDP glucuronosyltransferase 1 family, polypeptide A complex locus), PRKCA (protein kinase C, alpha), COMT (catechol-.beta.-methyltransferase), S100B (S100 calcium binding protein B), EGR1 (early growth response 1), PRL (prolactin), IL15 (interleukin 15), DRD4 (dopamine receptor D4), CAMK2G (calcium/calmodulin-dependent protein kinase II gamma), SLC22A2 (solute carrier family 22 (organic cation transporter), member 2), CCL11 (chemokine (C—C motif) ligand 11), PGF (B321 placental growth factor), THPO (thrombopoietin), GP6 (glycoprotein VI (platelet)), TACR1 (tachykinin receptor 1), NTS (neurotensin), HNF1A (HNF1 homeobox A), SST (somatostatin), KCND1 (potassium voltage-gated channel, Shal-related subfamily, member 1), LOC646627 (phospholipase inhibitor), TBXAS1 (thromboxane A synthase 1 (platelet)), CYP2J2 (cytochrome P450, family 2, subfamily J, polypeptide 2), TBXA2R (thromboxane A2 receptor), ADH1C (alcohol dehydrogenase 1C (class I), gamma polypeptide), ALOX12 (arachidonate 12-lipoxygenase), AHSG (alpha-2-HS-glycoprotein), BHMT (betaine-homocysteine methyltransferase), GJA4 (gap junction protein, alpha 4, 37 kDa), SLC25A4 (solute carrier family 25 (mitochondrial carrier; adenine nucleotide translocator), member 4), ACLY (ATP citrate lyase), ALOX5AP (arachidonate 5-lipoxygenase-activating protein), NUMA1 (nuclear mitotic apparatus protein 1), CYP27B1 (cytochrome P450, family 27, subfamily B, polypeptide 1), CYSLTR2 (cysteinyl leukotriene receptor 2), SOD3 (superoxide dismutase 3, extracellular), LTC4S (leukotriene C4 synthase), UCN (urocortin), GHRL (ghrelin/obestatin prepropeptide), APOC2 (apolipoprotein C-II), CLEC4A (C-type lectin domain family 4, member A), KBTBD10 (kelch repeat and BTB (POZ) domain containing 10), TNC (tenascin C), TYMS (thymidylate synthetase), SHC1 (SHC (Src homology 2 domain containing) transforming protein 1), LRP1 (low density lipoprotein receptor-related protein 1), SOCS3 (suppressor of cytokine signaling 3), ADH1B (alcohol dehydrogenase 1B (class I), beta polypeptide), KLK3 (kallikrein-related peptidase 3), HSD11B1 (hydroxysteroid (11-beta) dehydrogenase 1), VKORC1 (vitamin K epoxide reductase complex, subunit 1), SERPINB2 (serpin peptidase inhibitor, clade B (ovalbumin), member 2), TNS1 (tensin 1), RNF19A (ring finger protein 19A), EPOR (erythropoietin receptor), ITGAM (integrin, alpha M (complement component 3 receptor 3 subunit)), PITX2 (paired-like homeodomain 2), MAPK7 (mitogen-activated protein kinase 7), FCGR3A (Fc fragment of IgG, low affinity 111a, receptor (CD16a)), LEPR (leptin receptor), ENG (endoglin), GPX1 (glutathione peroxidase 1), GOT2 (glutamic-oxaloacetic transaminase 2, mitochondrial (aspartate aminotransferase 2)), HRH1 (histamine receptor H1), NR112 (nuclear receptor subfamily 1, group I, member 2), CRH (corticotropin releasing hormone), HTR1A (5-hydroxytryptamine (serotonin) receptor 1A), VDAC1 (voltage-dependent anion channel 1), HPSE (heparanase), SFTPD (surfactant protein D), TAP2 (transporter 2, ATP-binding cassette, sub-family B (MDR/TAP)), RNF123 (ring finger protein 123), PTK2B (PTK2B protein tyrosine kinase 2 beta), NTRK2 (neurotrophic tyrosine kinase, receptor, type 2), IL6R (interleukin 6 receptor), ACHE (acetylcholinesterase (Yt blood group)), GLP1R (glucagon-like peptide 1 receptor), GHR (growth hormone receptor), GSR (glutathione reductase), NQO1 (NAD(P)H dehydrogenase, quinone 1), NR5A1 (nuclear receptor subfamily 5, group A, member 1), GJB2 (gap junction protein, beta 2, 26 kDa), SLC9A1 (solute carrier family 9 (sodium/hydrogen exchanger), member 1), MAOA (monoamine oxidase A), PCSK9 (proprotein convertase subtilisin/kexin type 9), FCGR2A (Fc fragment of IgG, low affinity Ha, receptor (CD32)), SERPINF1 (serpin peptidase inhibitor, clade F (alpha-2 antiplasmin, pigment epithelium derived factor), member 1), EDN3 (endothelin 3), DHFR (dihydrofolate reductase), GAS6 (growth arrest-specific 6), SMPD1 (sphingomyelin phosphodiesterase 1, acid lysosomal), UCP2 (uncoupling protein 2 (mitochondrial, proton carrier)), TFAP2A (transcription factor AP-2 alpha (activating enhancer binding protein 2 alpha)), C4BPA (complement component 4 binding protein, alpha), SERPINF2 (serpin peptidase inhibitor, clade F (alpha-2 antiplasmin, pigment epithelium derived factor), member 2), TYMP (thymidine phosphorylase), ALPP (alkaline phosphatase, placental (Regan isozyme)), CXCR2 (chemokine (C—X—C motif) receptor 2), SLC39A3 (solute carrier family 39 (zinc transporter), member 3), ABCG2 (ATP-binding cassette, sub-family G (WHITE), member 2), ADA (adenosine deaminase), JAK3 (Janus kinase 3), HSPA1A (heat shock 70 kDa protein 1A), FASN (fatty acid synthase), FGF1 (fibroblast growth factor 1 (acidic)), F11 (coagulation factor XI), ATP7A (ATPase, Cu++ transporting, alpha polypeptide), CR1 (complement component (3b/4b) receptor 1 (Knops blood group)), GFAP (glial fibrillary acidic protein), ROCK1 (Rho-associated, coiled-coil containing protein kinase 1), MECP2 (methyl CpG binding protein 2 (Rett syndrome)), MYLK (myosin light chain kinase), BCHE (butyrylcholinesterase), LIPE (lipase, hormone-sensitive), PRDXS (peroxiredoxin 5), ADORA1 (adenosine A1 receptor), WRN (Werner syndrome, RecQ helicase-like), CXCR3 (chemokine (C—X—C motif) receptor 3), CD81 (CD81 molecule), SMAD7 (SMAD family member 7), LAMC2 (laminin, gamma 2), MAP3K5 (mitogen-activated protein kinase kinase kinase 5), CHGA (chromogranin A (parathyroid secretory protein 1)), IAPP (islet amyloid polypeptide), RHO (rhodopsin), ENPP1 (ectonucleotide pyrophosphatase/phosphodiesterase 1), PTHLH (parathyroid hormone-like hormone), NRG1 (neuregulin 1), VEGFC (vascular endothelial growth factor C), ENPEP (glutamyl aminopeptidase (aminopeptidase A)), CEBPB (CCAAT/enhancer binding protein (C/EBP), beta), NAGLU (N-acetylglucosaminidase, alpha-), F2RL3 (coagulation factor II (thrombin) receptor-like 3), CX3CL1 (chemokine (C-X3-C motif) ligand 1), BDKRB1 (bradykinin receptor B1), ADAMTS13 (ADAM metallopeptidase with thrombospondin type 1 motif, 13), ELANE (elastase, neutrophil expressed), ENPP2 (ectonucleotide pyrophosphatase/phosphodiesterase 2), CISH (cytokine inducible SH2-containing protein), GAST (gastrin), MYOC (myocilin, trabecular meshwork inducible glucocorticoid response), ATP1A2 (ATPase, Na+/K+ transporting, alpha 2 polypeptide), NF1 (neurofibromin 1), GJB1 (gap junction protein, beta 1, 32 kDa), MEF2A (myocyte enhancer factor 2A), VCL (vinculin), BMPR2 (bone morphogenetic protein receptor, type II (serine/threonine kinase)), TUBB (tubulin, beta), CDC42 (cell division cycle 42 (GTP binding protein, 25 kDa)), KRT18 (keratin 18), HSF1 (heat shock transcription factor 1), MYB (v-myb myeloblastosis viral oncogene homolog (avian)), PRKAA2 (protein kinase, AMP-activated, alpha 2 catalytic subunit), ROCK2 (Rho-associated, coiled-coil containing protein kinase 2), TFPI (tissue factor pathway inhibitor (lipoprotein-associated coagulation inhibitor)), PRKG1 (protein kinase, cGMP-dependent, type I), BMP2 (bone morphogenetic protein 2), CTNND1 (catenin (cadherin-associated protein), delta 1), CTH (cystathionase (cystathionine gamma-lyase)), CTSS (cathepsin S), VAV2 (vav 2 guanine nucleotide exchange factor), NPY2R (neuropeptide Y receptor Y2), IGFBP2 (insulin-like growth factor binding protein 2, 36 kDa), CD28 (CD28 molecule), GSTA1 (glutathione S-transferase alpha 1), PPIA (peptidylprolyl isomerase A (cyclophilin A)), APOH (apolipoprotein H (beta-2-glycoprotein I)), S100A8 (S100 calcium binding protein A8), IL11 (interleukin 11), ALOX15 (arachidonate 15-lipoxygenase), FBLN1 (fibulin 1), NR1H3 (nuclear receptor subfamily 1, group H, member 3), SCD (stearoyl-CoA desaturase (delta-9-desaturase)), GIP (gastric inhibitory polypeptide), CHGB (chromogranin B (secretogranin 1)), PRKCB (protein kinase C, beta), SRD5A1 (steroid-5-alpha-reductase, alpha polypeptide 1 (3-oxo-5 alpha-steroid delta 4-dehydrogenase alpha 1)), HSD11B2 (hydroxysteroid (11-beta) dehydrogenase 2), CALCRL (calcitonin receptor-like), GALNT2 (UDP-N-acetyl-alpha-D-galactosamine: polypeptide N-acetylgalactosaminyltransferase 2 (GalNAc-T2)), ANGPTL4 (angiopoietin-like 4), KCNN4 (potassium intermediate/small conductance calcium-activated channel, subfamily N, member 4), PIK3C2A (phosphoinositide-3-kinase, class 2, alpha polypeptide), HBEGF (heparin-binding EGF-like growth factor), CYP7A1 (cytochrome P450, family 7, subfamily A, polypeptide 1), HLA-DRBS (major histocompatibility complex, class II, DR beta 5), BNIP3 (BCL2/adenovirus E1B 19 kDa interacting protein 3), GCKR (glucokinase (hexokinase 4) regulator), S100A12 (S100 calcium binding protein A12), PADI4 (peptidyl arginine deiminase, type IV), HSPA14 (heat shock 70 kDa protein 14), CXCR1 (chemokine (C—X—C motif) receptor 1), H19 (H19, imprinted maternally expressed transcript (non-protein coding)), KRTAP19-3 (keratin associated protein 19-3), IDDM2 (insulin-dependent diabetes mellitus 2), RAC2 (ras-related C3 botulinum toxin substrate 2 (rho family, small GTP binding protein Rac2)), RYR1 (ryanodine receptor 1 (skeletal)), CLOCK (clock homolog (mouse)), NGFR (nerve growth factor receptor (TNFR superfamily, member 16)), DBH (dopamine beta-hydroxylase (dopamine beta-monooxygenase)), CHRNA4 (cholinergic receptor, nicotinic, alpha 4), CACNA1C (calcium channel, voltage-dependent, L type, alpha 1C subunit), PRKAG2 (protein kinase, AMP-activated, gamma 2 non-catalytic subunit), CHAT (choline acetyltransferase), PTGDS (prostaglandin D2 synthase 21 kDa (brain)), NR1H2 (nuclear receptor subfamily 1, group H, member 2), TEK (TEK tyrosine kinase, endothelial), VEGFB (vascular endothelial growth factor B), MEF2C (myocyte enhancer factor 2C), MAPKAPK2 (mitogen-activated protein kinase-activated protein kinase 2), TNFRSF11A (tumor necrosis factor receptor superfamily, member 11a, NFKB activator), HSPA9 (heat shock 70 kDa protein 9 (mortalin)), CYSLTR1 (cysteinyl leukotriene receptor 1), MAT1A (methionine adenosyltransferase I, alpha), OPRL1 (opiate receptor-like 1), IMPA1 (inositol(myo)-1(or 4)-monophosphatase 1), CLCN2 (chloride channel 2), DLD (dihydrolipoamide dehydrogenase), PSMA6 (proteasome (prosome, macropain) subunit, alpha type, 6), PSMB8 (proteasome (prosome, macropain) subunit, beta type, 8 (large multifunctional peptidase 7)), CHI3L1 (chitinase 3-like 1 (cartilage glycoprotein-39)), ALDH1B1 (aldehyde dehydrogenase 1 family, member B1), PARP2 (poly (ADP-ribose) polymerase 2), STAR (steroidogenic acute regulatory protein), LBP (lipopolysaccharide binding protein), ABCC6 (ATP-binding cassette, sub-family C(CFTR/MRP), member 6), RGS2 (regulator of G-protein signaling 2, 24 kDa), EFNB2 (ephrin-B2), GJB6 (gap junction protein, beta 6, 30 kDa), APOA2 (apolipoprotein A-II), AMPD1 (adenosine monophosphate deaminase 1), DYSF (dysferlin, limb girdle muscular dystrophy 2B (autosomal recessive)), FDFT1 (farnesyl-diphosphate farnesyltransferase 1), EDN2 (endothelin 2), CCR6 (chemokine (C—C motif) receptor 6), GJB3 (gap junction protein, beta 3, 31 kDa), IL1RL1 (interleukin 1 receptor-like 1), ENTPD1 (ectonucleoside triphosphate diphosphohydrolase 1), BBS4 (Bardet-Biedl syndrome 4), CELSR2 (cadherin, EGF LAG seven-pass G-type receptor 2 (flamingo homolog, Drosophila)), F11R (F11 receptor), RAPGEF3 (Rap guanine nucleotide exchange factor (GEF) 3), HYAL1 (hyaluronoglucosaminidase 1), ZNF259 (zinc finger protein 259), ATOX1 (ATX1 antioxidant protein 1 homolog (yeast)), ATF6 (activating transcription factor 6), KHK (ketohexokinase (fructokinase)), SAT1 (spermidine/spermine N1-acetyltransferase 1), GGH (gamma-glutamyl hydrolase (conjugase, folylpolygammaglutamyl hydrolase)), TIMP4 (TIMP metallopeptidase inhibitor 4), SLC4A4 (solute carrier family 4, sodium bicarbonate cotransporter, member 4), PDE2A (phosphodiesterase 2A, cGMP-stimulated), PDE3B (phosphodiesterase 3B, cGMP-inhibited), FADS1 (fatty acid desaturase 1), FADS2 (fatty acid desaturase 2), TMSB4X (thymosin beta 4, X-linked), TXNIP (thioredoxin interacting protein), LIMS1 (LIM and senescent cell antigen-like domains 1), RHOB (ras homolog gene family, member B), LY96 (lymphocyte antigen 96), FOXO1 (forkhead box 01), PNPLA2 (patatin-like phospholipase domain containing 2), TRH (thyrotropin-releasing hormone), GJC1 (gap junction protein, gamma 1, 45 kDa), SLC17A5 (solute carrier family 17 (anion/sugar transporter), member 5), FTO (fat mass and obesity associated), GJD2 (gap junction protein, delta 2, 36 kDa), PSRC1 (proline/serine-rich coiled-coil 1), CASP12 (caspase 12 (gene/pseudogene)), GPBAR1 (G protein-coupled bile acid receptor 1), PXK (PX domain containing serine/threonine kinase), IL33 (interleukin 33), TRIB1 (tribbles homolog 1 (Drosophila)), PBX4 (pre-B-cell leukemia homeobox 4), NUPR1 (nuclear protein, transcriptional regulator, 1), 15-Sep (15 kDa selenoprotein), CILP2 (cartilage intermediate layer protein 2), TERC (telomerase RNA component), GGT2 (gamma-glutamyltransferase 2), MT-CO1 (mitochondrially encoded cytochrome c oxidase I), and UOX (urate oxidase, pseudogene). In an additional embodiment, the chromosomal sequence may further be selected from Pon1 (paraoxonase 1), LDLR (LDL receptor), ApoE (Apolipoprotein E), Apo B-100 (Apolipoprotein B-100), ApoA (Apolipoprotein(a)), ApoA1 (Apolipoprotein A1), CBS (Cystathione B-synthase), Glycoprotein IIb/IIb, MTHRF (5,10-methylenetetrahydrofolate reductase (NADPH), and combinations thereof. In one iteration, the chromosomal sequences and proteins encoded by chromosomal sequences involved in cardiovascular disease may be chosen from Cacna1C, Sod1, Pten, Ppar(alpha), Apo E, Leptin, and combinations thereof. The text herein accordingly provides exemplary targets as to CRISPR or CRISPR-Cas systems or complexes.

The following are incorporated by reference.
Biosecurity Implications of Gene Drive Research (http://nas-sites.org/gene-drives/2015/10/07/implications-of-gene-drive-research-on-biosecurity-webinar/). You, E.
Biotechnology. A prudent path forward for genomic engineering and germline gene modification. Baltimore, D.; Berg, P.; Botchan, M.; Carroll, D.; Charo, R. A.; Church, G.; Corn, J. E.; Daley, G. Q.; Doudna, J. A.; Fenner, M.; Greely, H. T.; Jinek, M.; Martin, G. S.; Penhoet, E.; Puck, J.; Sternberg, S. H.; Weissman, J. S.; Yamamoto, K. R. Science 2015, 348, 36-8. PMC4394183
BIOSAFETY. Safeguarding gene drive experiments in the laboratory. Akbari, O. S.; Bellen, H. J.; Bier, E.; Bullock, S. L.; Burt, A.; Church, G. M.; Cook, K. R.; Duchek, P.; Edwards, O. R.; Esvelt, K. M.; Gantz, V. M.; Golic, K. G.; Gratz, S. J.; Harrison, M. M.; Hayes, K. R.; James, A. A.; Kaufman, T. C.; Knoblich, J.; Malik, H. S.; Matthews, K. A.; O'Connor-Giles, K. M.; Parks, A. L.; Perrimon, N.; Port, F.; Russell, S.; Ueda, R.; Wildonger, J. Science 2015, 349, 927-9. PMC4692367
Gene drive overdrive. Nat Biotech 2015, 33, 1019-1021
Opinion: Is CRISPR-based gene drive a biocontrol silver bullet or global conservation threat? Webber, B. L.; Raghu, S.; Edwards, O. R. Proceedings of the National Academy of Sciences 2015, 112, 10565-10567
Gene drives spread their wings. Saey, T. H. Science News 2015, 188, 16
Concerning RNA-guided gene drives for the alteration of wild populations. Esvelt, K. M.; Smidler, A. L.; Catteruccia, F.; Church, G. M. eLife 2014, 3, e03401
The dawn of active genetics. Gantz, V. M.; Bier, E. Bioessays 2016, 38, 50-63
Cheating evolution: engineering gene drives to manipulate the fate of wild populations. Champer, J.; Buchman, A.; Akbari, O. S. Nat Rev Genet 2016, 17, 146-159
Entomological terrorism: a tactic in assymmetrical warfare. Monthei, D.; Mueller, S.; Lockwood, J.; Debboun, M. US Army Med Dep J 2010, 11-21
CRISPR-mediated direct mutation of cancer genes in the mouse liver. Xue, W.; Chen, S.; Yin, H.; Tammela, T.; Papagiannakopoulos, T.; Joshi, N. S.; Cai, W.; Yang, G.; Bronson, R.; Crowley, D. G.; Zhang, F.; Anderson, D. G.; Sharp, P. A.; Jacks, T. Nature 2014, 514, 380-4. PMC4199937
Rapid modelling of cooperating genetic events in cancer through somatic genome editing. Sanchez-Rivera, F. J.; Papagiannakopoulos, T.; Romero, R.; Tammela, T.; Bauer, M. R.; Bhutkar, A.; Joshi, N. S.; Subbaraj, L.; Bronson, R. T.; Xue, W.; Jacks, T. Nature 2014, 516, 428-31. PMC4292871
CRISPR-Cas9 knockin mice for genome editing and cancer modeling. Platt, R. J.; Chen, S.; Zhou, Y.; Yim, M. J.; Swiech, L.; Kempton, H. R.; Dahlman, J. E.; Parnas, O.; Eisenhaure, T. M.; Jovanovic, M.; Graham, D. B.; Jhunjhunwala, S.; Heidenreich, M.; Xavier, R. J.; Langer, R.; Anderson, D. G.; Hacohen, N.; Regev, A.; Feng, G.; Sharp, P. A.; Zhang, F. Cell 2014, 159, 440-55. PMC4265475
Global microRNA depletion suppresses tumor angiogenesis. Chen, S.; Xue, Y.; Wu, X.; Le, C.; Bhutkar, A.; Bell, E. L.; Zhang, F.; Langer, R.; Sharp, P. A. Genes Dev 2014, 28, 1054-67. PMC4035535
Applications of the CRISPR-Cas9 system in cancer biology. Sánchez-Rivera, F. J.; Jacks, T. Nat Rev Cancer 2015, 15, 387-95
Genome-wide CRISPR screen in a mouse model of tumor growth and metastasis. Chen, S.; Sanjana, N. E.; Zheng, K.; Shalem, O.; Lee, K.; Shi, X.; Scott, D. A.; Song, J.; Pan, J. Q.; Weissleder, R.; Lee, H.; Zhang, F.; Sharp, P. A. Cell 2015, 160, 1246-60. PMC4380877
Increasing the efficiency of precise genome editing with CRISPR-Cas9 by inhibition of nonhomologous end joining. Maruyama, T.; Dougan, S. K.; Truttmann, M. C.; Bilate, A. M.; Ingram, J. R.; Ploegh, H. L. Nat Biotech 2015, 33, 538-542
Increasing the efficiency of homology-directed repair for CRISPR-Cas9-induced precise gene editing in mammalian cells. Chu, V. T.; Weber, T.; Wefers, B. Nat. Biotech 2015, 33, 543-8
Enhancing homology-directed genome editing by catalytically active and inactive CRISPR-Cas9 using asymmetric donor DNA. Richardson, C. D.; Ray, G. J.; DeWitt, M. A.; Curie, G. L.; Corn, J. E. Nat Biotech 2016, 34, 339-344
The cytotoxicity of (−)-lomaiviticin A arises from induction of double-strand breaks in DNA. Colis, L. C.; Woo, C. M.; Hegan, D. C.; Li, Z.; Glazer, P. M.; Herzon, S. B. Nature chemistry 2014, 6, 504-510
Structural basis for DNA cleavage by the potent antiproliferative agent (−)-lomaiviticin A. Woo, C. M.; Li, Z.; Paulson, E. K. 2016, 113, 2851-6
Rational design of human DNA ligase inhibitors that target cellular DNA replication and repair. Chen, X.; Zhong, S.; Zhu, X.; Dziegielewska, B.; Ellenberger, T.; Wilson, G. M.; MacKerell, A. D., Jr.; Tomkinson, A. E. Cancer Res 2008, 68, 3169-77. PMC2734474
An inhibitor of nonhomologous end joining abrogates double-strand break repair and impedes cancer progression. Srivastava, M.; Nambiar, M.; Sharma, S.; Karki, S. S.; Goldsmith, G.; Hegde, M.; Kumar, S.; Pandey, M.; Singh, R. K.; Ray, P.; Natarajan, R.; Kelkar, M.; De, A.; Choudhary, B.; Raghavan, S. C. Cell 2012, 151, 1474-87
Increasing the efficiency of precise genome editing with CRISPR-Cas9 by inhibition of nonhomologous end joining. Maruyama, T.; Dougan, S. K.; Truttmann, M. C.; Bilate, A. M.; Ingram, J. R.; Ploegh, H. L. Nat Biotechnol 2015, 33, 538-42. PMC4618510
Increasing the efficiency of homology-directed repair for CRISPR-Cas9-induced precise gene editing in mammalian cells. Chu, V. T.; Weber, T.; Wefers, B.; Wurst, W.; Sander, S.; Rajewsky, K.; Kuhn, R. Nat Biotechnol 2015, 33, 543-8
SCR7 is neither a selective nor a potent inhibitor of human DNA ligase IV. Greco, G. E.; Matsumoto, Y.; Brooks, R. C.; Lu, Z.; Lieber, M. R.; Tomkinson, A. E. DNA Repair (Amst) 2016, 43, 18-23. PMC5042453
A chemical compound that stimulates the human homologous recombination protein RAD51. Jayathilaka, K.; Sheridan, S. D.; Bold, T. D.; Bochenska, K.; Logan, H. L.; Weichselbaum, R. R.; Bishop, D. K.; Connell, P. P. Proc Natl Acad Sci USA 2008, 105, 15848-53. PMC2572930
Dual and Opposite Effects of hRAD51 Chemical Modulation on HIV-1 Integration. Thierry, S.; Benleulmi, M. S.; Sinzelle, L.; Thierry, E.; Calmels, C.; Chaignepain, S.; Waffo-Teguo, P.; Merillon, J. M.; Budke, B.; Pasquet, J. M.; Litvak, S.; Ciuffi, A.; Sung, P.; Connell, P.; Hauber, I.; Hauber, J.; Andreola, M. L.; Delelis, O.; Parissi, V. Chem Biol 2015, 22, 712-23. PMC4889029
Nuclear domain ‘knock-in’ screen for the evaluation and identification of small molecule enhancers of CRISPR-based genome editing. Pinder, J.; Salsman, J.; Dellaire, G. Nucleic Acids Res 2015, 43, 9379-92. PMC4627099
A guide to genome engineering with programmable nucleases. Kim, H.; Kim, J. S. Nat Rev Genet 2014, 15, 321-34
Synthetic mimetics of protein secondary structure domains. Ross, N. T.; Katt, W. P.; Hamilton, A. D. Philosophical transactions. Series A, Mathematical, physical, and engineering sciences 2010, 368, 989-1008
The crystal structure of TAL effector PthXol bound to its DNA target. Mak, A. N.-S.; Bradley, P.; Cernadas, R. A.; Bogdanove, A. J.; Stoddard, B. L. Science (New York, N.Y.) 2012, 335, 716-719
Calculating structures and free energies of complex molecules: combining molecular mechanics and continuum models. Kollman, P. A.; Massova, I.; Reyes, C.; Kuhn, B.; Huo, S.; Chong, L.; Lee, M.; Lee, T.; Duan, Y.; Wang, W. Accounts of chemical research 2000, 33, 889-897%@ 0001-4842
Antechamber: an accessory software package for molecular mechanical calculations. Wang, J.; Wang, W.; Kollman, P. A.; Case, D. A. J. Am. Chem. Soc 2001, 222, U403
Boronate-mediated biologic delivery. Ellis, G. A.; Palte, M. J.; Raines, R. T. J Am Chem Soc 2012, 134, 3631-4. Pmc3304437
Cell-penetrating peptides: 20 years later, where do we stand? Bechara, C.; Sagan, S. FEBS Lett 2013, 587, 1693-702
Click chemistry in complex mixtures: bioorthogonal bioconjugation. McKay, C. S.; Finn, M. G. Chem Biol 2014, 21, 1075-101. Pmc4331201
High-frequency off-target mutagenesis induced by CRISPR-Cas nucleases in human cells. Fu, Y.; Foden, J. A.; Khayter, C.; Maeder, M. L.; Reyon, D.; Joung, J. K.; Sander, J. D. Nat Biotechnol 2013, 31, 822-6. Pmc3773023
Multidimensional chemical control of CRISPR-Cas9. Maji, B.; Moore, C. L.; Zetsche, B.; Volz, S. E.; Zhang, F.; Shoulders, M. D.; Choudhary, A. Nat Chem Biol 2016, advance online publication
Evolution and classification of the CRISPR-Cas systems. Makarova, K. S.; Haft, D. H.; Barrangou, R.; Brouns, S. J.; Charpentier, E.; Horvath, P.; Moineau, S.; Mojica, F. J.; Wolf, Y. I.; Yakunin, A. F.; van der Oost, J.; Koonin, E. V. Nat Rev Microbiol 2011, 9, 467-77. PMC3380444
An updated evolutionary classification of CRISPR-Cas systems. Makarova, K. S.; Wolf, Y. I.; Alkhnbashi, O. S.; Costa, F.; Shah, S. A.; Saunders, S. J.; Barrangou, R.; Brouns, S. J.; Charpentier, E.; Haft, D. H.; Horvath, P.; Moineau, S.; Mojica, F. J.; Terns, R. M.; Terns,
M. P.; White, M. F.; Yakunin, A. F.; Garrett, R. A.; van der Oost, J.; Backofen, R.; Koonin, E. V. Nat Rev Microbiol 2015
Advances in CRISPR-Cas9 genome engineering: lessons learned from RNA interference. Barrangou, R.; Birmingham, A.; Wiemann, S.; Beijersbergen, R. L.; Hornung, V.; Smith, A. Nucleic Acids Res 2015, 43, 3407-19. PMC4402539
The mechanism of double-strand DNA break repair by the nonhomologous DNA end-joining pathway. Lieber, M. R. Annu Rev Biochem 2010, 79, 181-211. Pmc3079308
Reduced ciliary polycystin-2 in induced pluripotent stem cells from polycystic kidney disease patients with PKD1 mutations. Freedman, B. S.; Lam, A. Q.; Sundsbak, J. L.; Iatrino, R.; Su, X.; Koon, S. J.; Wu, M.; Daheron, L.; Harris, P. C.; Zhou, J.; Bonventre, J. V. J Am Soc Nephrol 2013, 24, 1571-86. Pmc3785271
Polycystic kidney disease. Wilson, P. D. N Engl J Med 2004, 350, 151-64
Fibrocystin/polyductin, found in the same protein complex with polycystin-2, regulates calcium responses in kidney epithelia. Wang, S.; Zhang, J.; Nauli, S. M.; Li, X.; Starremans, P. G.; Luo, Y.; Roberts, K. A.; Zhou, J. Mol Cell Biol 2007, 27, 3241-52. Pmc1899915
Site-specific C-terminal and internal loop labeling of proteins using sortase-mediated reactions. Guimaraes, C. P.; Witte, M. D.; Theile, C. S.; Bozkurt, G.; Kundrat, L.; Blom, A. E.; Ploegh, H. L. Nat Protoc 2013, 8, 1787-99. PMC3943461
Site-specific N-terminal labeling of proteins using sortase-mediated reactions. Theile, C. S.; Witte, M. D.; Blom, A. E.; Kundrat, L.; Ploegh, H. L.; Guimaraes, C. P. Nat Protoc 2013, 8, 1800-7. PMC3941705
Sortase-mediated ligations for the site-specific modification of proteins. Schmohl, L.; Schwarzer, D. Curr Opin Chem Biol 2014, 22, 122-8
A split-Cas9 architecture for inducible genome editing and transcription modulation. Zetsche, B.; Volz, S. E.; Zhang, F. Nat Biotechnol 2015, 33, 139-42. PMC4503468
Rational design of a split-Cas9 enzyme complex. Wright, A. V.; Sternberg, S. H.; Taylor, D. W.; Staahl, B. T.; Bardales, J. A.; Kornfeld, J. E.; Doudna, J. A. Proc Natl Acad Sci USA 2015, 112, 2984-9. PMC4364227
Rationally engineered Cas9 nucleases with improved specificity. Slaymaker, I. M.; Gao, L.; Zetsche, B.; Scott, D. A.; Yan, W. X.; Zhang, F. Science (New York, N.Y.) 2016, 351, 84-88
RNA-programmed genome editing in human cells. Jinek, M.; East, A.; Cheng, A.; Lin, S.; Ma, E.; Doudna, J. Elife 2013, 2, e00471. PMC3557905
Cationic lipid-mediated delivery of proteins enables efficient protein-based genome editing in vitro and in vivo. Zuris, J. A.; Thompson, D. B.; Shu, Y.; Guilinger, J. P.; Bessen, J. L.; Hu, J. H.; Maeder, M. L.; Joung, J. K.; Chen, Z. Y.; Liu, D. R. Nat Biotechnol 2015, 33, 73-80. PMC4289409
High-frequency off-target mutagenesis induced by CRISPR-Cas nucleases in human cells. Fu, Y.; Foden, J. A.; Khayter, C.; Maeder, M. L.; Reyon, D.; Joung, J. K.; Sander, J. D. Nat Biotechnol 2013, 31, 822-6. Pmc3773023
DNA repair targeted therapy: The past or future of cancer treatment? Gavande, N. S.; VanderVere-Carozza, P. S.; Hinshaw, H. D.; Jalal, S. I.; Sears, C. R.; Pawelczak, K. S.; Turchi, J. J. Pharmacol Ther 2016, 160, 65-83. PMC4811676

### EXAMPLES

### Example 1—Effector Components

### Diazotization

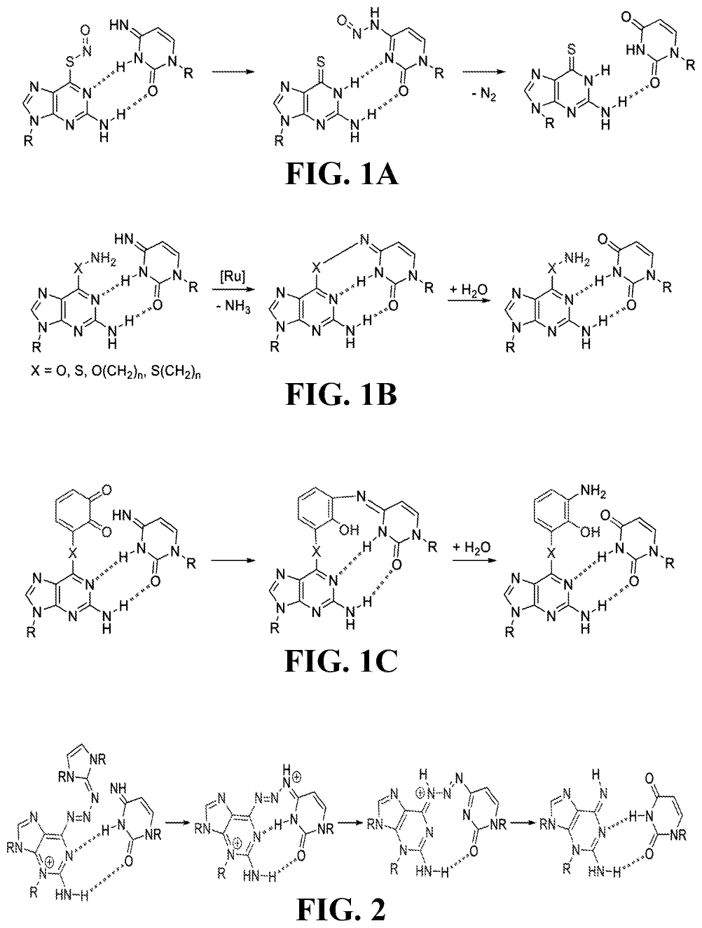

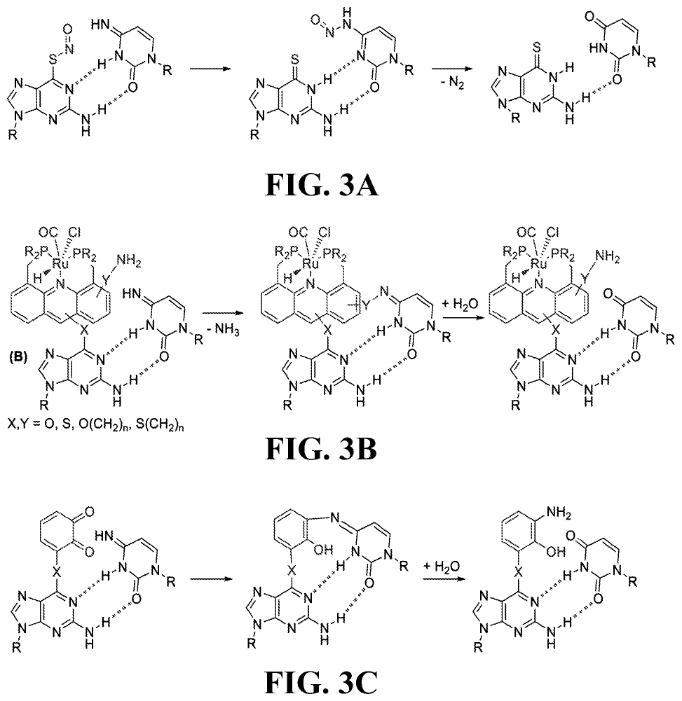

In 2004 Ali at el. reported the first example of sequence- and base-specific delivery of nitric oxide from thioguanosine to cytosine. (1) It is the first step of diazotization reaction which is usually followed by deprotonation of NH, protonation of O, elimination of hydroxyl with formation of diazonium ion and, finally, nucleophilic substitution of N₂ with water and formation of cytosine (FIG. 1A). (2) Since Ali's ODN wasn't equipped with base and/or acid to perform efficient proton transfer, yield of deamination was only 8%. PNAs can be designed to display various functionalities which are known to facilitate diazotization, such as saccharin, (3) sulfonic acids, (4, 5) or certain nucleophiles.(6) Saccharin stabilizes NO by the formation of salt with an efficient charge delocalization, when methanesulfonic and paratoluenesulfonic acids act as mild proton donors. In addition to this, structure of the NO-delivery component can be varied or sulfur can be substituted with other element, such as selenium or oxygen. (7, 8, 9, 10, 11) Facilitators of diazotization may also include nucleophiles comprising sulfites, bisulfites, thiols, selenides, phosphates, phosphites, phosphides, chloride, bromide, iodide, thiocyanate and their analogs.

### Triazabutadiene,

Alternatively, diazonium ion can be generated from the certain triazabutadienes upon protonation. (12) Again, we will rely on the base-pairing between imine form of cytosine and diazonium donor obtained from 4-aminoguanine. N1 alkylation of starting triazabutadiene will facilitate protonation of nitrogen attached to guanine and formation of desired diazonium of cytosine (FIG. 2). Hydrolysis of imines and oximes of cytosine can lead to uracyl. (13) Ruthenium (II) hydride complexes are known for their ability to catalyze generation of imine intermediates from two amines. (14) Displaying this efficient hydrogen transfer catalysts right next to primary amine-containing guanine on a PNA strain or other DNA reader can provide effective catalysis. The length and nature of connectors X will vary for the achievement of the highest reactivity and selectivity, and can be further tested and may depend on strain content, location of strain, among other factors. Upon base pairing, Ruthenium will catalyze coupling of amine donor with cytosine with formation of imine and elimination of ammonia. However, there is a risk that on the next step reduction of imine instead of hydrolysis will take place (FIG. 1B). Likely, in the recent example of Ru catalyzed deamination of primary amines nucleophilic attack of imine by water was much faster than other alternative pathways. (15) Ru-complexes are actively used for cell imaging probes and in the development of therapeutics, nevertheless its cytotoxicity should be considered. (16) Applicants believe that Ruthenium conjugation to PNA will secure low concentration and specificity of action (similar to selective nitrosation in Ali's work).

Finally, following Golime's deamination approach, 1,2-diketonecyclodiene derivative will be conjugated to PNA (FIG. 1C). (17) NH2-group of cytosine will undergo condensation with one of the carbonyls and following prototropic rearrangement (driven by aromatization) will generate ortho-hydroxy-containing imine of aniline. Next OH assisted hydrolyzes will produce desired product of deamination and 2-aminophenol derivative. This transformation will require stoichiometric amounts of 1,2-diketo reagent or it might be recycled if oxidation catalyst will be attached to the nearby position of PNA.

A known method for the cytosine deamination is bisulfite catalyzed hydrolysis, however, this reaction suffers from very harsh conditions, which can be potentially modulated (e.g. addition of quaternary amines). (18) Mechanism of cytosine activation with bisulfite includes nucleophilic attack on C6 position with disruption of aromaticity.

Interesting, but alkylation of cytosine with epoxides also leads to partial or complete deamination. (19) Reaction of 2-deoxycytidine with 1-chloroethenyloxirane gives N3-alkylated 2-deoxyuridine, with 36% after a reaction time of 6 days. Similar deamination takes place upon treatment of either adenine or cytosine with styrene oxide (20, 21) The main challenges of this approach are high sensitivity of epoxides towards nucleophilic attacks and irreversible alkylation of N3 position of deaminated product.

Finally, some studies demonstrated that under UV-light deamination of cytosine is accelerated. (22) This acceleration was attributed to the photochemical formation of cyclobutane-containing dimers or cytosine hydrates. More recently, deamination was coupled with 5-carboxyvinyldeoxyuridine-mediated ligation photobranching what lead to clean site-selective transformation of cytosine to uracil. (23) However, in the initial report elevated temperatures were required for deamination. In the following work inosine was utilized as a counter base; it demonstrated more efficient hydrogen-bonding pattern and promoted deamination in the physiological conditions. (24) One more photo ligation reagent developed by Fujimoto and co-workers utilizes 3-cyanovinylcarbazole containing nucleoside, but it also suffers from the requirement of high temperature. (25, 26) Introduction of free right next to target cytosine activates the incoming nucleophile through hydrogen bonding with the water molecule facilitating the nucleophilic attack on C-4 carbon of cytosine and accelerating deamination reaction. (27)

The following references apply to Example 1
(1) Sequence- and base-specific delivery of nitric oxide to cytidine and 5-methylcytidine leading to efficient deamination. Ali, M. M.; Alam, R.; Kawasaki, T.; Nakayama, S.; Nagatsugi, F.; Sasaki, S. J. Am. Chem. Soc. 2004, 126, 8864-8865.
(2) Mechanism of nitric oxide induced deamination of cytosine Labet, V.; Grand, A.; Morell, C.; Cadeta, J.; Eriksson, L. A. Phys. Chem. Chem. Phys. 2009, 11, 2379-2386.
(3) Saccharin: an efficient organocatalyst for the one-pot synthesis of 4-amidocinnolines under metal and halogen-free conditions. Khaligh, N. G.; Mihankhah, T.; Johan, M. R.; Ching, J. J. Chemical Monthly, 2018, 149, 1083-1087.
(4) Telescopic synthesis of azo compounds via stable arenediazonium tosylates by using n-butyl nitriteas diazotization reagent. Khaligh, N. G.; Hamid, S. B. A.; Johari S. Polycyclic Aromatic Compounds, 2017, doi: 10.1080/10406638.2017.1326951.
(5) Unprecedented substoichiometric use of hazardous aryl diazonium salts in the Heck-Matsuda reaction via a double catalytic cycle. Le Callonnec, F.; Fouquet, E.; Felpin, F.-X. et. al. Org. Lett. 2011, 13, 2646-2649.
(6) Effect of added nucleophilic species on the rate of primary amino acid nitrosation. da Silva, G.; Kennedy, E. M. Dlugogorskida, B. Z. J. Am. Chem. Soc. 2005, 127, 3664-3665.
(7) 4-Aryl-1,3,2-oxathiazolylium-5-olates as pH-Controlled NO-Donors: The Next Generation of S-Nitrosothiols. Lu, D.; Nadas, J.; Zhang, G.; Johnson, W.; Zweier, J. L.; Cardounel, A. J.; Villamena, F. A.; Wang, P. G. J. Am. Chem. Soc. 2007, 129, 5503-5514.
(8) Seleno-Nucleobases and their water-soluble ruthenium-arene half-sandwich complexes: chemistry and biological activity. Mitra, R.; Pramanik, A. K.; Samuelson, A. G. Eur. J. Inorg. Chem. 2014, 5733-5740.
(9) Site-directed delivery of nitric oxide to cancers. Sharma, K.; Chakrapani, H. Nitric Oxide, 2014, 43, 8-16.
(10) Chemistry of the nitric oxide-releasing diazeniumdiolate (“Nitrosohydroxylamine”) functional group and its oxygen-substituted derivatives Hrabbie et al. Chem. Rev. 2002, 102, 1135-1154.
(11) Progress towards clinical application of the nitric oxide-releasing diazeniumdiolates. Keefer et al. Annu. Rev. Pharmacol. Toxicol. 2003, 43, 585-607.
(12) Water-soluble triazabutadienes that release diazonium species upon protonation under physiologically relevant conditions. Kimani, F. W.; Jewett, J. C. Angew. Chem. Int. Ed. 2015, 54, 4051-4054.
(13) Transformation of cytosine to uracil in single-stranded DNA via their oxime sulfonates. Oka, Y.; Takeia, F.; Nakatani, K. Oka et. al. Chem. Commun. 2010, 46, 3378-3380.
(14) Ruthenium-catalyzed deaminative redistribution of primary and secondary amines. Kostera, S.; Wyrzykiewicz, B.; Pawluć, P.; Marciniec, B. Dalton Trans. 2017, 46, 11552.
(15) Direct deamination of primary amines by water to produce alcohols. Khusnutdinova, J. R.; Ben-David, Y.; Milstein, D. Angew. Chem. Int. Ed. 2013, 52, 6269-6272.
(16) Ruthenium (II) polypyridyl complexes and DNA—from structural probes to cellular imaging and therapeutics. Gill, M. R.; Thomas, J. A. Chem. Soc. Rev. 2012, 41, 3179-3192.
(17) Biomimetic oxidative deamination catalysis via ortho-naphthoquinone-catalyzed aerobic oxidation strategy. Golime, G.; Bogonda, G.; Kim, H. Y.; Oh, K. ACS Catal. 2018, 8, 4986-4990.
(18) Bisulfite-mediated deamination of cytosine in DNA under near-neutral conditions. Hayatsu, H.; Negishi, K.; Suzuki, T.; Wataya, Y. Genes and Environment, 2011, 33, 66-70.
(19) Identification of adducts derived from reactions of (1-chloroethenyl)oxirane with nucleosides and calf thymus DNA. Munter, T.; Cottrell, L.; Hill, S.; Kronberg, L.; Watson, W. P.; Golding B. T. Chem. Res. Toxicol. 2002, 15, 1549-1560.
(20) Deamination and Dimroth rearrangement of deoxyadenosine-styrene oxide adducts in DNA. Barlow, T.; Takeshita, J.; Dipple, A. Chem. Res. Toxicol. 1998, 11, 838-845.
(21) Investigation of hydrolytic deamination of 1-(2-hydroxy-1-phenylethyl)adenosine. Barlow, T.; Ding, J.; Vouros, P.; Dipple, A. Barlow et al. Chem. Res. Toxicol. 1997, 10, 1247-1249.
(22) Accelerated deamination of cytosine residues in UV-induced cyclobutane pyrimidine dimers leads to CC→TT transitions. Peng, W.; Shaw, B. R. Biochemistry 1996, 35, 10172-10181.
(23) Site-specific transition of cytosine to uracil via reversible DNA photoligation. Fujimoto, K.; Matsuda, S.; Yoshimura, Y.; Matsumura, T.; Hayashic, M.; Saito, I. Chem. Commun. 2006, 3223-3225.
(24) Effect of nucleobase change on cytosine deamination through DNA photo-cross-linking reaction via 3-cyanovinylcarbazole nucleoside. Fujimoto et. al. Mol. BioSyst. 2017, 13, 1152.
(25) Fujimoto et. al. Chem. Commun. 2010, 46, 7545-7547.
(26) Fujimoto et. al. ChemBioChem. 2010, 11, 1661-1664.
(27) Fujimoto et. al. ChemBioChem. 2018, Accepted Article, Ultra-acceleration of Photochemical Cytosine Deamination by 5′-phosphate ODN probe containing 3-Cyanovinylcarbazole at 5′-end. doi: 10.1002/cbic.201800384

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. An engineered, non-naturally occurring nucleic acid modifying system, comprising:
a) one or more DNA readers, wherein a first engineered, non-naturally occurring DNA reader, binds a target nucleic acid; and
b) one or more effector components, wherein a first effector component is a small molecule and modifies the target nucleic acid.

2. The system of claim 1, wherein the first DNA reader is a peptide nucleic acid (PNA) polymer.

3. The system of claim 1, wherein the first effector component is a small molecule synthetic nuclease.

4. The system of claim 1, wherein the first effector component is a nitric oxide donor and optionally comprises a second effector component that facilitates diazotization.

5. The system of claim 4, wherein the first effector component comprises thioguanosine.

6. The system of claim 5, further comprising saccharin, sulfonic acids and/or other nucleophiles, comprising sulfites, bisulfites, thiols, selenides, phosphates, phosphites, phosphides, chloride, bromide, iodide, thiocyanate and their analogs.

7. The system of claim 1, wherein the first effector component is a diazonium ion donor.

8. The system of claim 7, wherein the first effector component comprises a triazabutadiene.

9. The system of claim 8, wherein the first effector component is a ruthenium catalyst, optionally, Ruthenium (II) hydride.

10. The system of claim 9, wherein ruthenium (II) hydride is conjugated to the DNA reader and further comprises an amine donor.

11. The system of claim 1, wherein the first effector is a 1,2-cyclodienone, 9,10-phenanthrenedione, 1,2-anthracenedione, 2,3-benzofurandione, indole-2,3-dione, 1,2-acenaphthylenedione and their derivatives.

12. The system of claim 11, further comprising a catalyst, optionally an oxidation catalyst.

13. The system of claim 12, wherein the catalyst is attached in close proximity on the first DNA reader, a second DNA reader, or on an optionally provided guide RNA.

14. The system of claim 1, wherein the first effector component is an epoxide.

15. The system of claim 1, wherein the first effector component is a bisulfite donor, optionally comprising a second effector component comprising a quarternary amine.

16. The system of claim 1, wherein the first effector component is a deaminator and further comprises UV light.

17. The system of claim 1 wherein the DNA reader comprises a PEG linker comprising one or more functional groups.

18. The system of claim 1, wherein the DNA reader comprises a linker comprising disulfide, products of azide/alkyne [3+2] cycloaddition, amide, carbamate, ester, urea, thiourea, for the attachment of the one or more effector components.

19. The system of claim 3, wherein the first effector component is linked to the first DNA reader.

20. The system of claim 7, wherein the first effector component is covalently linked to the first DNA reader.

21. The system of claim 1, further comprising a second DNA reader and a second effector component.

22. The system of claim 16, wherein the first effector component is covalently linked to the first DNA reader and the second effector component is covalently linked to the second DNA reader.

23. The system of claim 16, wherein both the first and second DNA readers are PNA polymers.

24. The system of claim 16, wherein the first effector component is an inactive small molecule synthetic nuclease and the second effector component is a trigger reagent, wherein the trigger reagent activates the small molecule synthetic nuclease.

25. The system of claim 1, wherein the synthetic nuclease is a single strand breaking small molecule.

26. The system of claim 1, wherein the one or more effector components comprises the first effector component, a second effector component, a third effector component, and a fourth effector component.

27. The system of claim 24, wherein the one or more DNA readers comprises the first DNA reader and a second DNA reader that are PNA polymers, and the first, second, third, and fourth effector component are small molecule single strand breaking synthetic nucleases.

28. The system of claim 25, wherein the first and second synthetic nucleases are linked to the first PNA polymer, and the third and fourth synthetic nucleases are linked to the second PNA polymer.

29. The system of any of the previous claims, further comprising one or more single-stranded oligo donors (ssODNs).

30. The system of any of the previous claims, further comprising one or more NHEJ inhibitors and/or one or more HDR activators.

31. The system of claim 31, wherein the NHEJ inhibitor is an inhibitor of DNA ligase IV, KU70, or KU80.

32. The system of claim 31, wherein the NHEJ inhibitor is a small molecule.

33. The system of claim 31, wherein the NHEJ inhibitor is selected from the group consisting of SCR7-G, KU inhibitor, and analogs thereof.

34. The system of claim 31, wherein the HDR activator is a small molecule.

35. The system of any of claims 1-32, wherein the target nucleic acid comprises chromosomal DNA.

36. The system of any of claims 1-32, wherein the target nucleic acid comprises mitochondrial DNA.

37. The system of any of claims 1-32, wherein the target nucleic acid comprises viral, bacterial, or fungal DNA.

38. The system of any of claims 1-32, wherein the target nucleic acid comprises viral, bacterial, or fungal RNA.

39. The system of any one of the preceding claims, further comprising a deamination enhancer, optionally wherein the enhancer is UV-light.

40. The system of any one of the preceding claims, further comprising a delivery enhancer.

41. The system of claim 38, wherein the delivery enhancer is a cellular permeability enhancer.

42. A method of precise genome editing in a cell or tissue, comprising delivering the system of any one of the previous claims to the cell or tissue.

43. The method of claim 40, wherein the system is delivered using nanoparticles.

44. The method of claim 41, wherein the nanoparticles are selected from poly(lactic co-glycolic acids) (PLGA) nanoparticles, lipid based nanoparticles, PLGA/PLA nanoparticles, mixed poly amine-PLA conjugate nanoparticles, cationic peptide nanoparticles, anionic peptide nanoparticles, or dendrimer based nanoparticles.


SEQUENCE LISTING (12 sequences)

SEQ ID NO: 1 - protein, 5 aa, Artificial Sequence
  Synthetic
GGGGS

SEQ ID NO: 2 - DNA, 12 bp, Artificial Sequence
  Synthetic
acttgtttaagt

SEQ ID NO: 3 - DNA, 16 bp, Artificial Sequence
  Synthetic
ggcaccgagtcggtgc

SEQ ID NO: 4 - protein, 15 aa, Artificial Sequence
  Synthetic
GGGGSGGGGSGGGGS

SEQ ID NO: 5 - protein, 30 aa, Artificial Sequence
  Synthetic
GGGGSGGGGSGGGGSGGGGSGGGGSGGGGS

SEQ ID NO: 6 - protein, 45 aa, Artificial Sequence
  Synthetic
GGGGSGGGGSGGGGSGGGGSGGGGSGGGGSGGGGSGGGGSGGGGS

SEQ ID NO: 7 - protein, 60 aa, Artificial Sequence
  Synthetic
GGGGSGGGGSGGGGSGGGGSGGGGSGGGGSGGGGSGGGGSGGGGSGGGGSGGGGSGGGGS

SEQ ID NO: 8 - DNA, 20 bp, Homo sapiens
gccaaattggacgaccctcg

SEQ ID NO: 9 - DNA, 20 bp, Homo sapiens
cgaggagacccccgtttcgg

SEQ ID NO: 10 - DNA, 20 bp, Homo sapiens
cgaggagacccccgtttcgg

SEQ ID NO: 11 - DNA, 20 bp, Homo sapiens
cccgccgccgccgtggctcg

SEQ ID NO: 12 - DNA, 20 bp, Homo sapiens
tgagctctacgagatccaca